(19) United States
(12) Patent Application Publication
(10) Pub. No.: US 2009/0209522 A1
(43) Pub. Date: Aug. 20, 2009

(54) Heterocyclic Non-Peptide GNRH Antagonists

(76) Inventors: Graham Andrew Showell, Cambridge (GB); David John Miller, Cambridge (GB); Angela Glen, Cambridge (GB); Maria Angeles Cubillo De Dios, Cambridge (GB); Kevin Merchant, Cambridge (GB); Ajay Kumar Mandal, Cambridge (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/993,863

(22) PCT Filed: Jun. 27, 2006
(86) PCT No.: PCT/GB06/02344

§ 371 Date: Jan. 15, 2009

(30) Foreign Application Priority Data
Jun. 28, 2005 (GB) 0513176.8
Oct. 19, 2005 (GB) 05212780.2
May 4, 2006 (GB) 0608846.2

(51) Int. Cl. A61K 31/551; C07D 413/14; A61K 31/5377; A61K 31/506; C07D 417/02; C07D 417/14; A61P 35/00

(52) U.S. Cl. 514/218; 544/122; 514/235.8; 514/256; 544/333; 540/575

(57) ABSTRACT

A compound of formula (I): wherein either B is absent and A and Z are the same or different and are each hydrogen, halogen, alkyl, hydroxy, alkoxy, —CN, —C(R^{c})₂OH, —N(R^{d})C(═X)R^{c}, —C(═X)N(R^{c})(R^{d}), —S(O)ₘ—R^{c}, —N(R^{c})(R^{d})S(O)₂, —S(O)₂N(R^{c})(R^{d}), —N(R^{c})₂, aryl optionally substituted with R^{a} or —O-aryl optionally substituted with R^{a}; or B is present and is —(CH₂)ₙ—, —C(R^{b})₂— or —O—, or B taken together with A or Z can be —C═C(R^{b})—, —C(R^{b})═C—, —CH₂—CH(R^{b})— or —CH(R^{b})—CH₂—; D is —O— or —S(O)_{m′}—; E is a bond or is —(CH₂)ₙ—, —N(R^{d})—, —(CH₂)ₙN(R^{d})— or —N(R^{d})(CH₂)ₙ—; F is —C(═X)—; G is —(CH₂)ₙ—, —N(R^{d})—, —(CH₂)ₙN(R^{d})— or —N(R^{d})(CH₂)ₙ; J is a bond, —O—, —N(R^{C})C(═X)—, —C(═X)N(R^{c})—, —S(O)_{m′}—, —N(R^{c})S(O)ₘ—, —S(O)ₙN(R^{c})—, —N(R^{c})— or —N(R^{g})(R^{h}); K is a bond, alkylene, cycloalkylene, cycloalkenylene, arylene, heterocycloalkylene, heterocycloalkylene or heteroarylene; and L is hydrogen or a terminal group; has therapeutic utility.

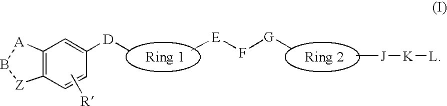

### FIELD OF THE INVENTION

This invention relates to compounds and their use in therapy.

### BACKGROUND TO THE INVENTION

Gonadotropin-Releasing Hormone (GnRH) plays a key role in the biology of reproduction. GnRH is also known as luteinizing hormone-releasing hormone (LH-RH).

The GnRH decapeptide (pyro-Glu-His-Trp-Ser-Tyr-Gly-Leu-Art-Pro-Gly-NH₂ or p-EHWSYGLRPG-NH₂) is formed in neurons of the medical basal hypothalamus from a larger precursor via enzymatic processing. The peptide is released in a pulsatile manner into the pituitary portal circulation system, where GnRH interacts with high-affinity receptors (7-transmembrane G-protein coupled receptors) in the anterior pituitary gland located at the base of the brain. Here, GnRH triggers the release of luteinizing hormone (LH) and follicle-stimulating hormone (FSH), both of which are gonadotropic hormones (gonadotropins). LH stimulates the production of testosterone and estradiol in the testes and ovaries respectively, whilst FSH stimulates follicle growth in women and sperm formation in men. When correctly functioning, the pulsatile release and concentration levels of GnRH are critical for the maintaining of gonadal steroidogenesis and for normal functions of reproduction related to growth and sexual development.

The pituitary response to GnRH varies greatly throughout life. GnRH and the gonadotropins first appear in the foetus at about ten weeks of gestation. Sensitivity to GnRH reduces until the onset of puberty. There is, however, a brief rise during the first three months after birth. Prior to puberty, the FSH response to GnRH is greater than that of LH. Once puberty begins, sensitivity to GnRH increases, and pulsatile LH secretion ensues. Later in puberty and throughout the reproductive years, pulsatile release of GnRH occurs throughout the day, with responsiveness to LH being greater than that of FSH. Pulsatile GnRH release results in pulsatile LH and FSH release and thus testosterone and estradiol release from the gonads. Post-menopause, the concentration of FSH and LH rise, and the post-menopausal levels of FSH are higher than those of LH.

Chronic administration of GnRH agonists and antagonists results in decreased circulating levels of both LH and FSH. GnRH agonists are compounds that mimic endogenous GnRH to stimulate receptors on the pituitary gland, resulting in release of LH and FSH. After a transient rise in gonadal hormone production (“flare” response), the chronic administration of GnRH agonists results in down-regulation of the GnRH receptors. This down-regulation and desensitization results in a reduction in the circulating levels of LH and FSH. In spite of the symptom-exacerbating hormonal flare experienced, GnRH agonists have been the preferred treatment for sex-steroid-dependent pathophysiologies. GnRH agonists have been used to reduce testosterone production, thereby reducing prostate volume in benign prostatic hyperplasia (BPH) and slowing tumour growth in prostate cancer. Such compounds have also been used in the treatment of breast and ovarian cancers.

In recent years, GnRH antagonists have become available for clinical evaluation, and have been shown to have an immediate effect on the pituitary but without the observed flare associated with agonists. Use of GnRH antagonists has been reported for the treatment of ovarian, breast and prostate cancers.

Other uses of antagonists include endometriosis (including endometriosis with pain), uterine myoma, ovarian and mammary cystic diseases (including polycystic ovarian disease), prostatic hypertrophy, amenorrhoea (e.g. secondary amenorrhoea), and precocious puberty. These compounds may also be useful in the symptomatic relief of premenstrual syndrome (PMS). Antagonists may also be useful to regulate the secretion of gonadotropins in male mammals to arrest spermatogenesis (e.g. as male contraceptives), and for treatment of male sex offenders. GnRH antagonists and agonists have been shown to have utility in treatments where a reversible suppression of the pituitary-gonadal axis is desired.

The presence of GnRH receptors on anterior pituitary cells and several tumour cell types offers the opportunity to develop drugs that act upon receptors to treat both hormone-dependent and hormone-independent cancers.

Conventionally, androgen deprivation has been the most effective systematic therapy for the treatment of metastatic carcinoma of the prostate. The prostate gland requires androgens for normal growth, maintenance, and function. Prostate cancer and benign prostate hyperplasia, however, are common in men and develop in an environment of continuous exposure to androgen. Utilizing a GnRH antagonist to interrupt the pituitary-gonadal axis reduces androgen production and results in tumour growth modulation.

GnRH antagonists may save a direct effect on tumour growth by blocking receptors on the tumour cells. For those cancer types that respond both to sex hormones and to GnRH directly, antagonists should be effective in slowing tumour growth by two mechanisms. Since GnRH receptors are present on many prostate and breast cancer cells, it has recently been proposed that GnRH antagonists may also be effective in treating non-hormone-dependent tumours. Recent literature examples indicate that GnRH receptors are present on a number of cancer cell lines. In particular, prostate, ovarian and breast cancers (see for example Montagnani et al., Arch. Ital, Urol. Androl. 1997, 69(4), 257-263; Jungwirth et al, Prostate 1997, 32(3), 164-172; Srkalovic et al., Int J. Oncol. 1998, 12(3), 489-498; Kottler et al., Int J. Cancer 1997, 71(4), 595-599.

Available GnRH antagonists have primarily been peptide analogues of GnRH (see, for example, WO93/03058). Peptide antagonists of peptide hormones have some potency but, the use of current peptide antagonists is often associated with problems because peptides are degraded by physiological enzymes and often poorly distributed within the organism being treated. They thus have a limited effectiveness as drugs.

WO00/20358 discloses non-peptide analogues of GnRH.

### SUMMARY OF THE INVENTION

A first aspect of the invention is a compound of formula (I):

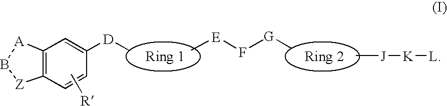

wherein

either B is absent and A and Z are the same or different and are each hydrogen, halogen, alkyl, hydroxy, alkoxy, —CN, —C(R^{c})₂OH, —N(R^{d})C(═X)R^{c}, —C(═X)N(R^{c})(R^{d}), —S(O)ₘ—R^{c}, —N(R^{c})(R^{d})S(O)₂, —S(O)₂N(R^{c})(R^{d}), —N(R^{e})₂, aryl optionally substituted with R^{a} or —O-aryl optionally substituted with R^{a}; or

B is present and is —(CH₂)ₙ—, —C(R^{b})₂— or —O—, or B taken together with A or Z can be —C═C(R^{b})—, —C(R^{b})═C—, —CH₂—CH(R^{b})— or —CH(R^{b})—CH₂—;

D is —O— or —S(O)ₘ—;

E is a bond or is —(CH₂)ₙ—, —N(R^{d})—, —(CH₂)ₙN(R^{d})— or N(R^{d})(CH₂)ₙ—;

F is —C(═X)—;

G is —(CH₂)ₙ—, —N(R^{d})—, —(CH₂)ₙN(R^{d})— or —N(R^{d})(CH₂)ₙ;

J is a bond or is —O—, —N(R^{c})C(═X)—, —C(═X)N(R^{c})—, —S(O)ₘ—, —N(R^{c})S(O)ₘ—, —S(O)ₘN(R^{c})—, —N(R^{e})— or —N(R^{g})(R^{h});

K is a bond or is alkylene optionally substituted with R^{b}; or K is cycloalkylene, cycloalkenylene, arylene, heterocycloalkylene, heterocycloalkylene or heteroarylene, any of which is optionally substituted with R^{a};

L is hydrogen, halogen, —N(R^{f})₂, —CN, —SO₂N(R^{a})₂, —SO₂N═C[N(R^{a})₂], —NHC(═O)NHOR^{a}, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}), —CN or a group comprising of

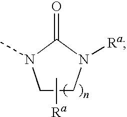

each R^{a} is the same or different and is hydrogen, halogen, alkyl, aryl, hydroxy, alkoxy, -alkoxy-(CH₂)ₙC(═O)OR^{b}, —O-aryl, —C(═X)R^{c}, —NO₂, —CN, —N(R^{c})C(═X)R^{c}, —C(═X)N(R^{c})₂, —S(O)₂N(R^{c})₂ or —N(R^{e})₂;

each R^{b} is the same or different and is hydrogen or alkyl;

each R^{c} is the same or different and is alkyl, cycloalkyl, -alkyl-aryl, -alkyl-cycloalkyl or aryl optionally substituted with R^{a};

each R^{d} is the same or different and is hydrogen or alkyl or aryl optionally with R^{a};

each R^{e} is the same or different and is hydrogen or alkyl; or R^{e} is aryl or heteroaryl, either of which is optionally substituted with R^{a};

each R^{f} is the same or different and is hydrogen or alkyl; or R^{f}—N—R^{f} taken together forms heterocycloalkyl, heterocycloalkenyl or heteroaryl, each of which is optionally substituted with R^{e};

each R^{g} is alkyl, cycloalkyl or alkyl-cycloalkyl, any of which is optionally substituted by an oxo and/or fluoro group;

each R^{h} is alkyl, cycloalkyl, or alkyl-cycloalkyl substituted with N(R^{f})₂; or R^{g} and R^{h} are taken together to form a heterocycloalkyl ring;

each X is the same or different and is oxygen or sulphur;

Ring 1 is a five- or six-membered heteroaryl ring containing at least 2 heteroatoms from O, N and/or S, which is optionally substituted with one or more R^{a};

Ring 2 is arylene or heteroarylene, either of which is optionally substituted with one or more R^{a};

each m is the same or different and is 0, 1 or 2; and

each n is the same or different and is 0, 1, 2 or 3;

or a pharmaceutically acceptable salt thereof.

Compounds of the invention may act as GnRH antagonists and, as a result, may have utility in cancer therapy or in the treatment or prevention of endometriosis, uterine myoma, an ovarian disease, a mammary cystic disease, prostatic hypertrophy, amenorrhoea, precocious puberty, premenstrual syndrome, a sex-steroid-dependent pathophysiology, benign prostatic hyperplasia or Alzheimer's disease, or to arrest spermatogenesis.

Accordingly, a second aspect of the invention is the use of a compound of the invention for the manufacture of a medicament for cancer therapy or for the treatment or prevention of endometriosis, uterine myoma, an ovarian disease, a mammary cystic disease, prostatic hypertrophy, amenorrhoea, precocious puberty, premenstrual syndrome, a sex-steroid-dependent pathophysiology, benign prostatic hyperplasia or Alzheimer's disease, or to arrest spermatogenesis.

Another aspect of the invention is a pharmaceutical composition comprising a compound of the invention and a pharmaceutically acceptable diluent or carrier.

### DESCRIPTION OF THE INVENTION

Certain compounds and combinations of substituents are preferred; in particular, see the subclaims.

The term “alkyl” as used herein refers to an optionally substituted straight or branched chain alkyl moiety having from one to six carbon atoms. The term includes, for example, methyl, ethyl, propyl, isopropyl, butyl, tert-butyl, pentyl, hexyl and the like. The substituents may be the same or different in each occurrence and selected from halogen and the like. “C₁₋₆ alkyl” has the same meaning. “Alkylene” refers to a similar, divalent group.

The term “alkoxy” as used herein refers to an optionally substituted straight or branched chain alkoxy group containing one to six carbon atoms. The term includes, for example, methoxy, ethoxy, propoxy, isopropoxy, butoxy, tert-butoxy, pentoxy, hexoxy and the like. The substituents may be the same or different in each occurrence and selected from halogen and the like. “C₁₋₆ alkoxy” has the same meaning.

The term “halogen” as used herein refers to F, Cl, Br or I.

The term “aryl” as used herein refers to optionally substituted aromatic ring systems comprising six to ten ring atoms, and optionally substituted polycyclic ring systems having two or more cyclic rings at least one of which is aromatic. This term includes, for example, phenyl and naphthyl. The group may be optionally substituted with the substituents being the same or different in each occurrence and selected from R^{a} and the like. “Arylene” refers to a similar, divalent group.

The term “cycloalkyl” as used herein refers to a saturated alicyclic moiety having from three to six carbon atoms. The term includes, for example, cyclopropyl, cyclobuty, cyclopentyl, cyclohexyl and the like. The group may be optionally substituted by any substituent described herein. “Cycloalkylene” refers to a similar, divalent group.

The term “cycloalkenyl” as used herein refers to an alicyclic moiety having from three to six carbon atoms and having in addition at least one double bond. The term includes, for example, cyclopentenyl, cyclohexenyl and the like. The group may be optionally substituted by any substituent described herein. “Cycloalkenylene” refers to a similar, divalent group.

The term “heterocycloalkyl” as used herein refers to a saturated heterocyclic moiety having from three to seven carbon atoms and one or more heteroatoms selected from the group N, O, S, P and Si. The term includes, for example, azetidinyl, pyrrolidinyl, tetrahydrofuranyl, piperidinyl and the like. The group may be optionally substituted by any substituent described herein. “Heterocycloalkylene” refers to a similar, divalent group.

The term “heteroaryl” as used herein refers to aromatic ring systems of five to ten atoms at least one atom of which is selected from O, N and S. The term includes, for example, furanyl, thiophenyl, pyridyl, indolyl, quinolyl and the like. The group may be optionally substituted with R^{a} and the like. “Heteroarylene” refers to a similar, divalent group.

Preferred compounds of the invention include those having the following formulae:

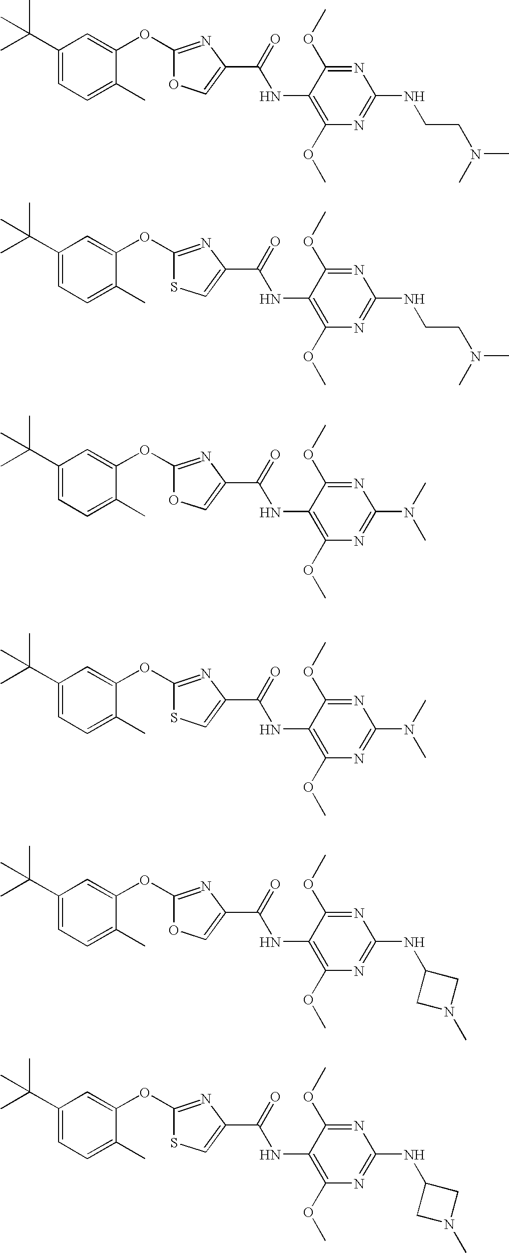

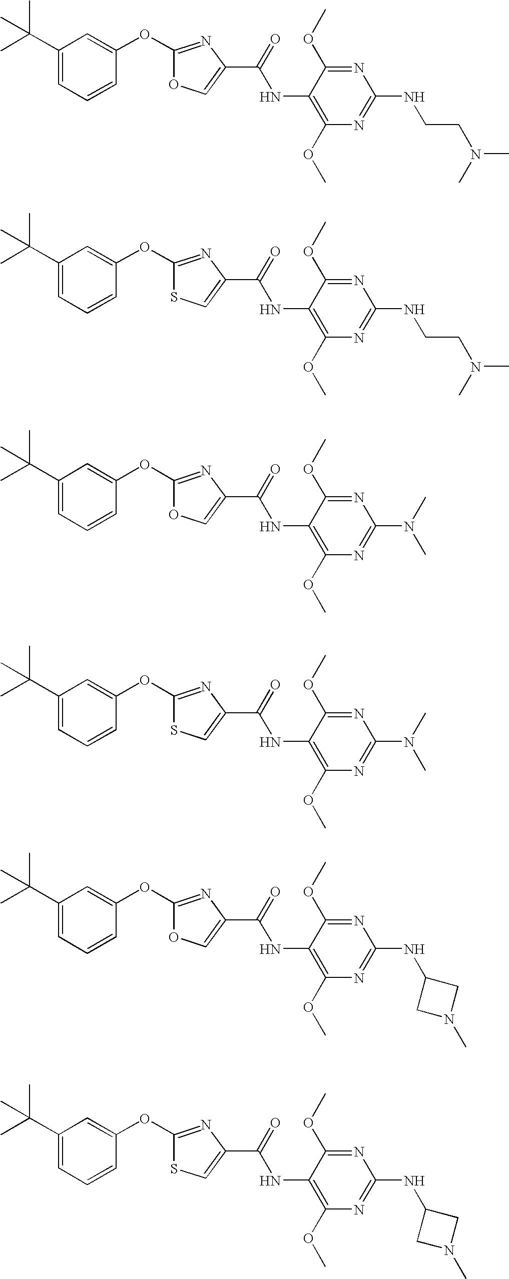

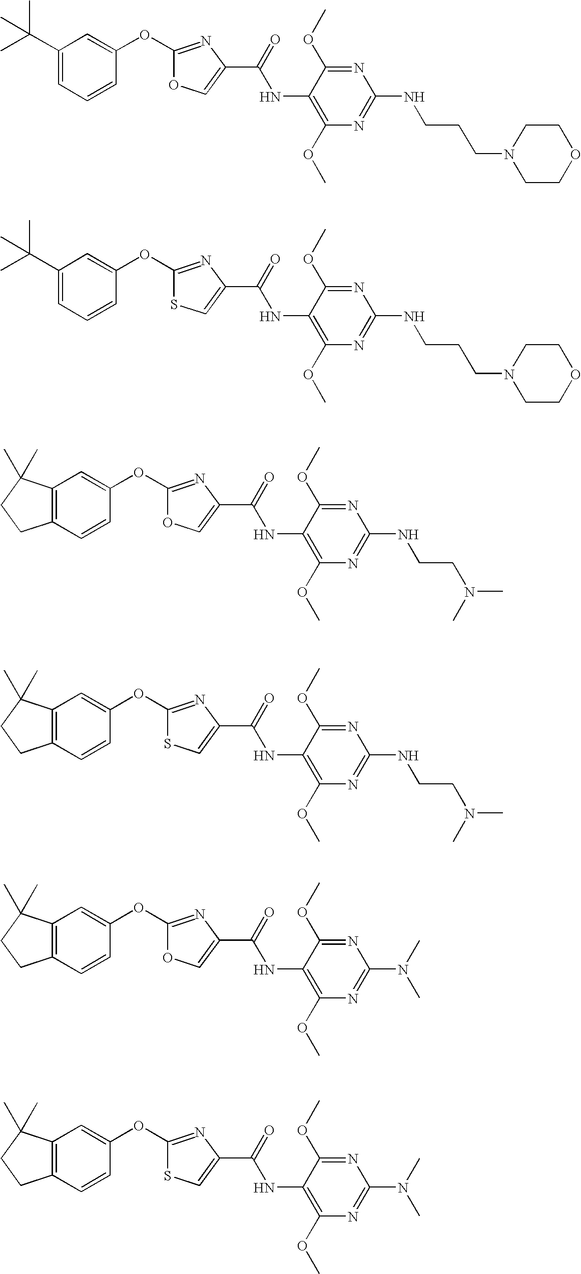

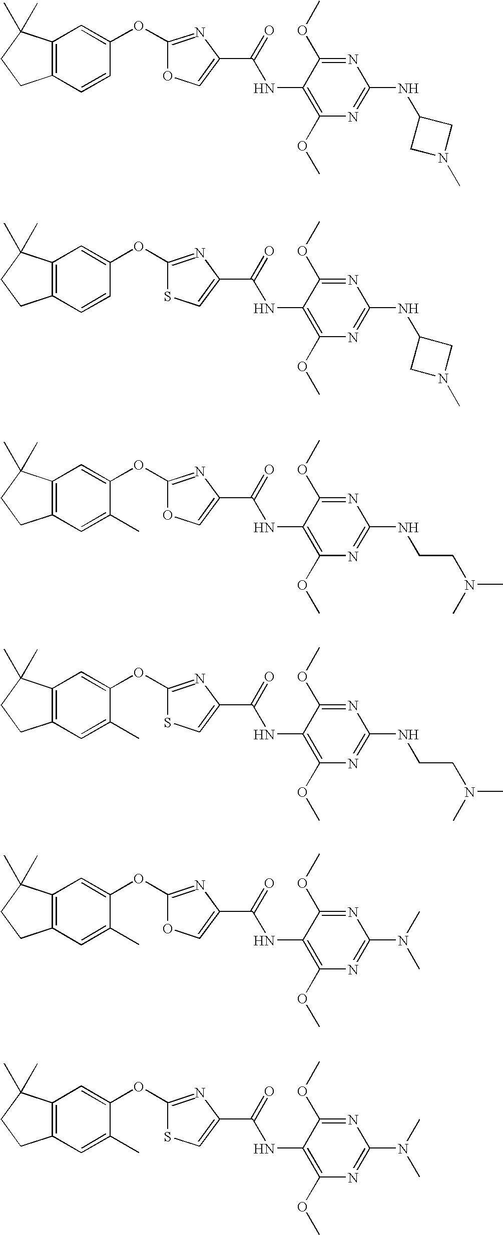

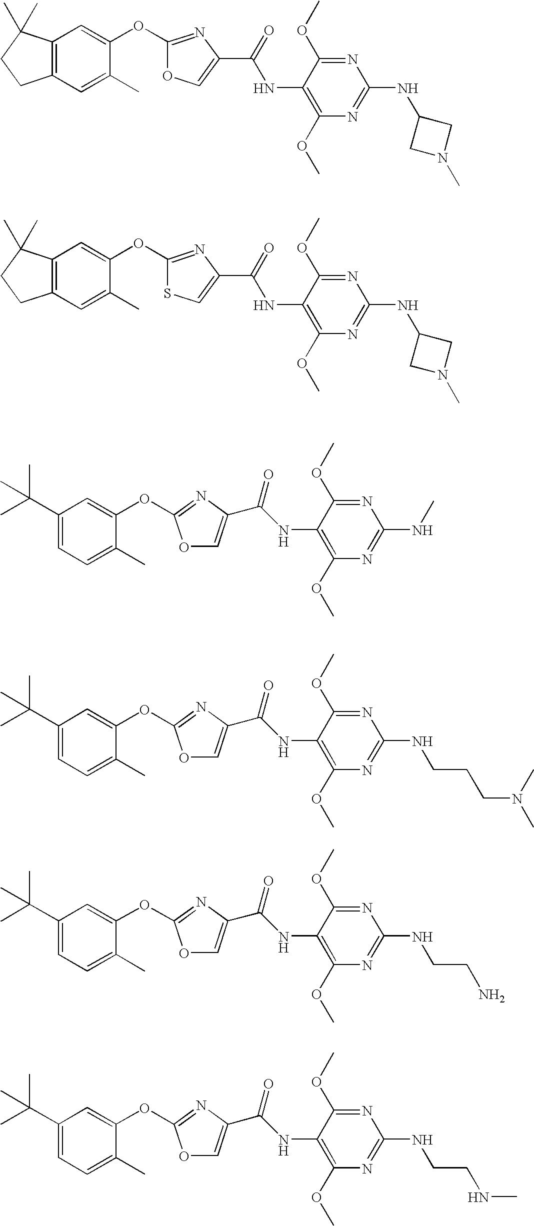

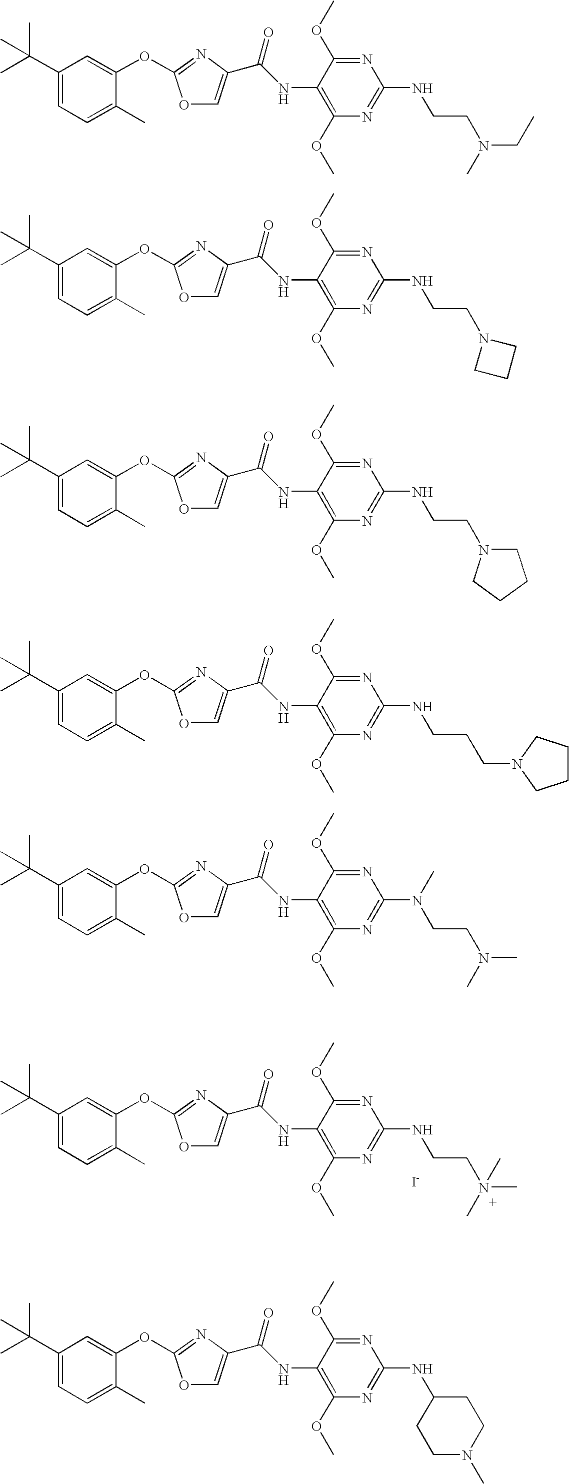

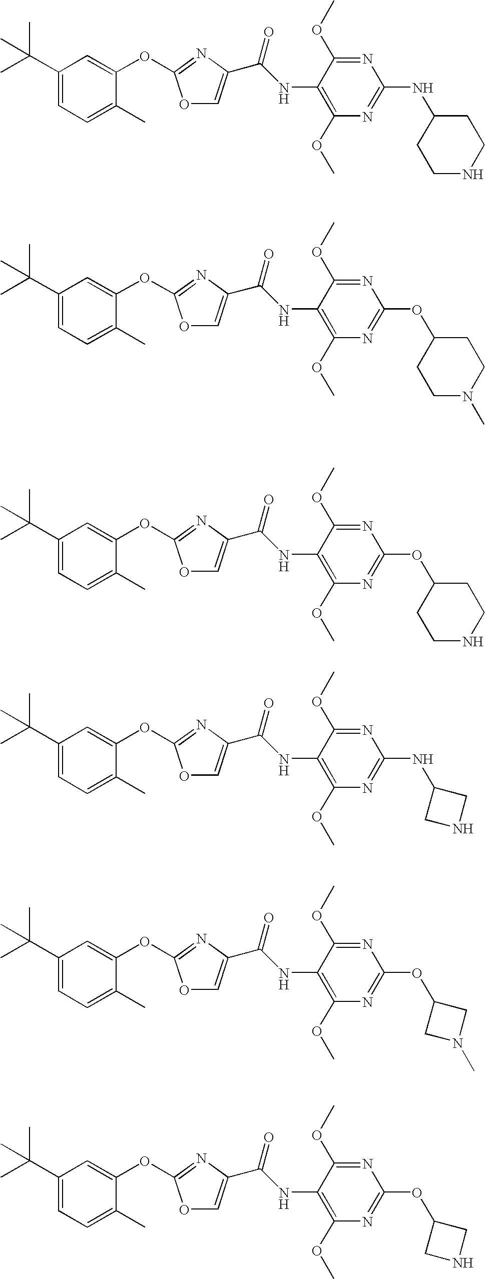

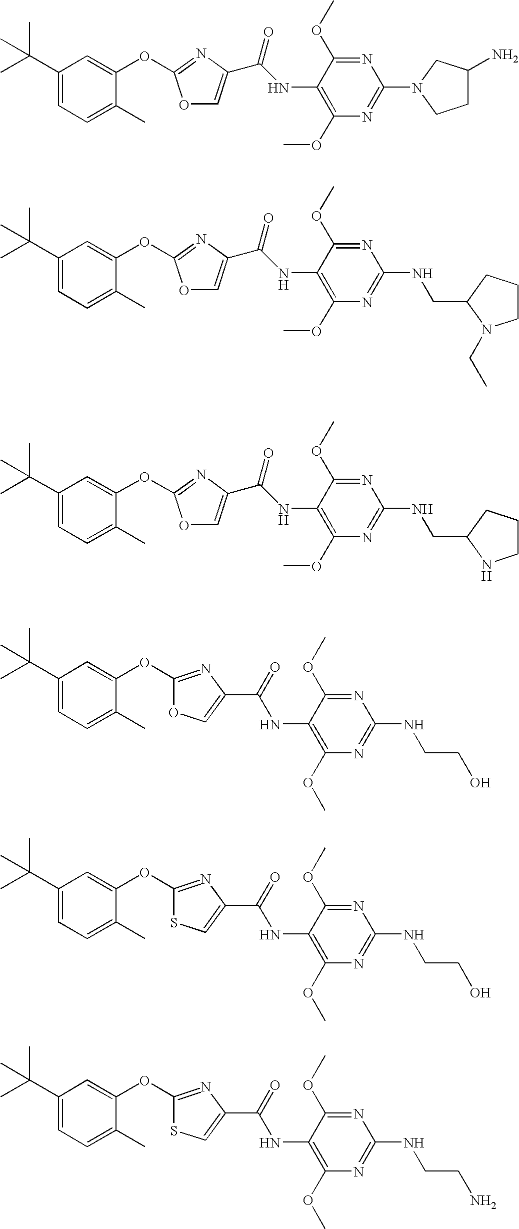

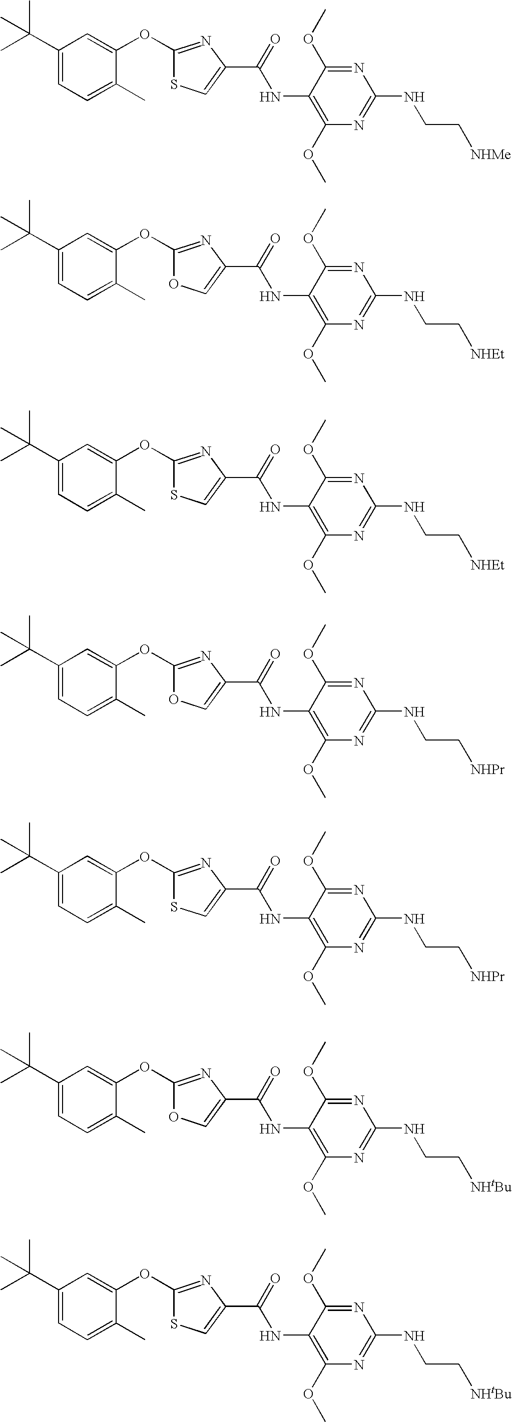

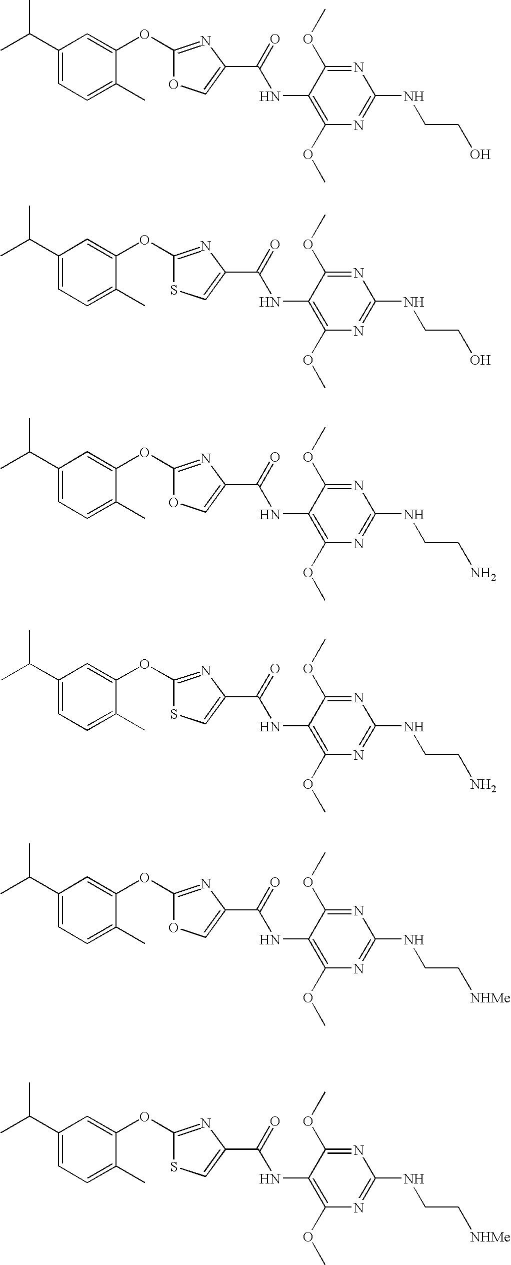

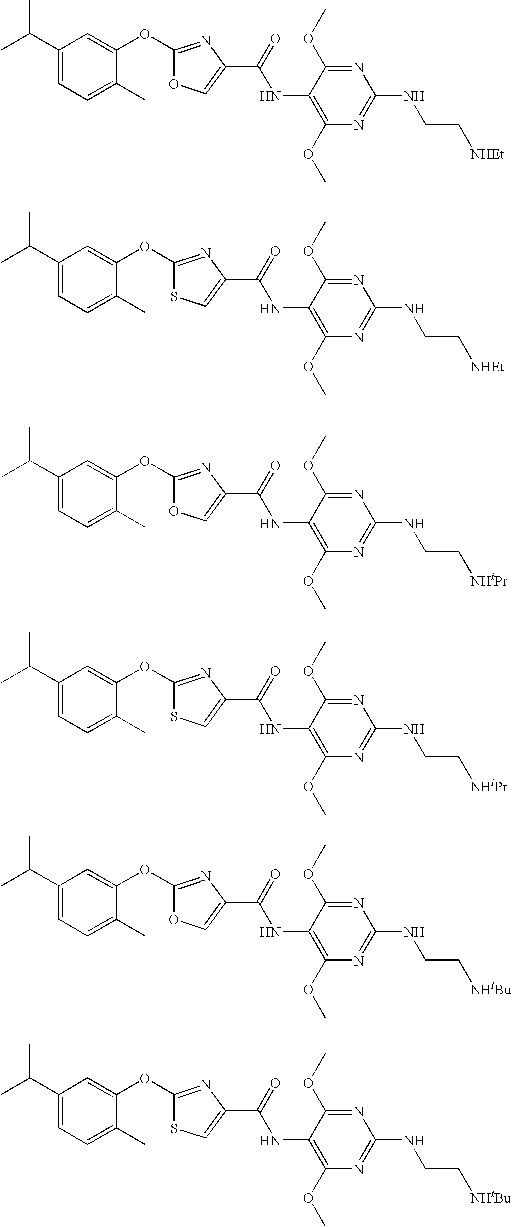

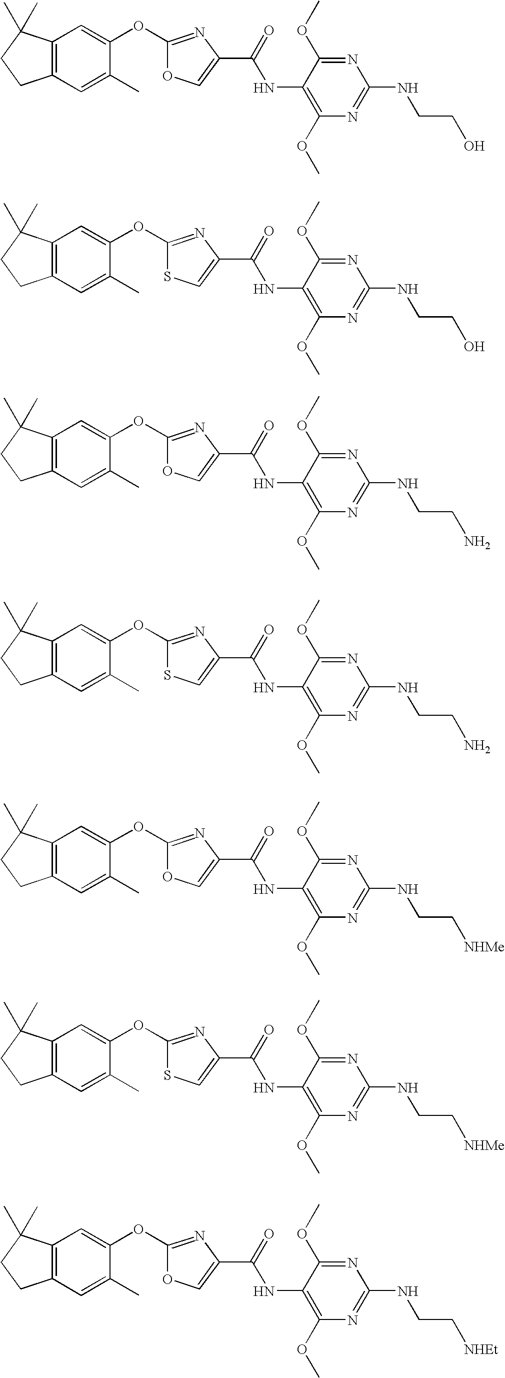

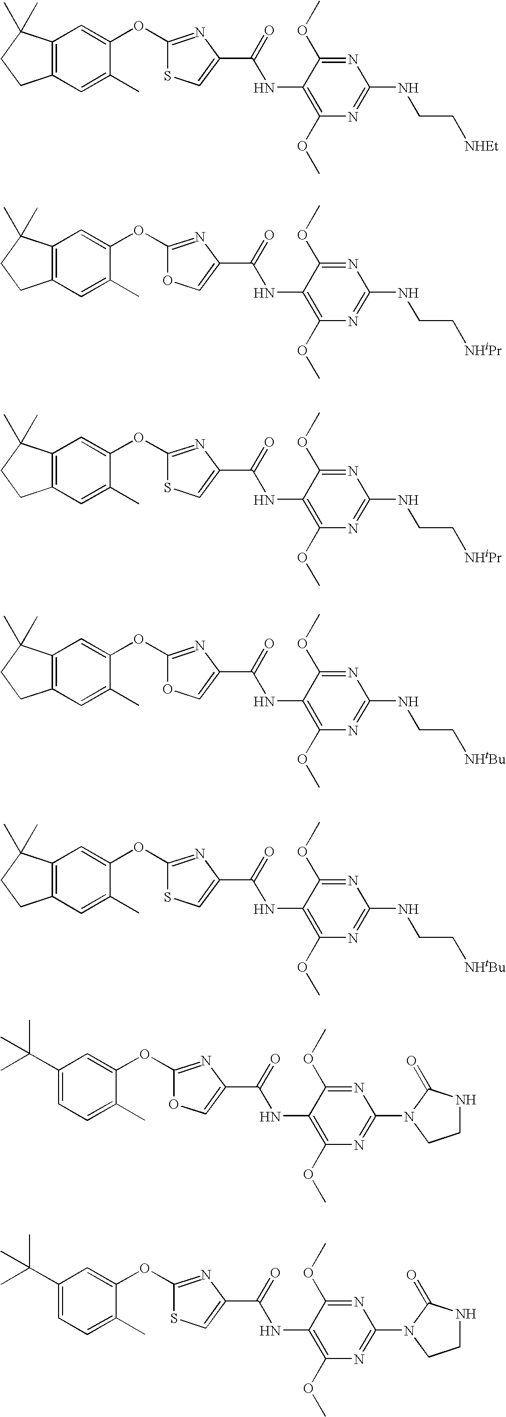

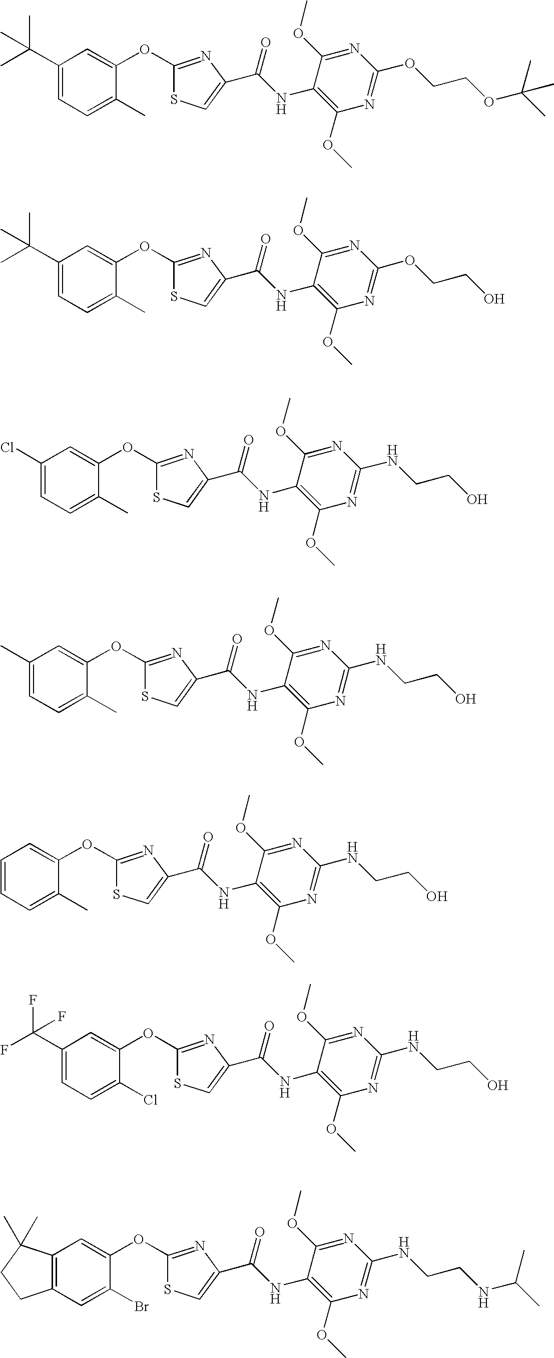

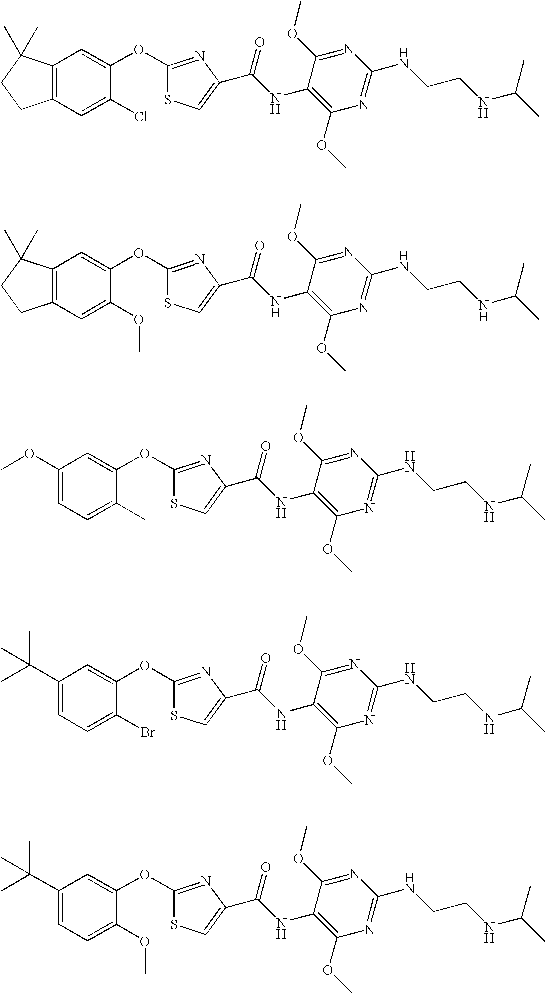

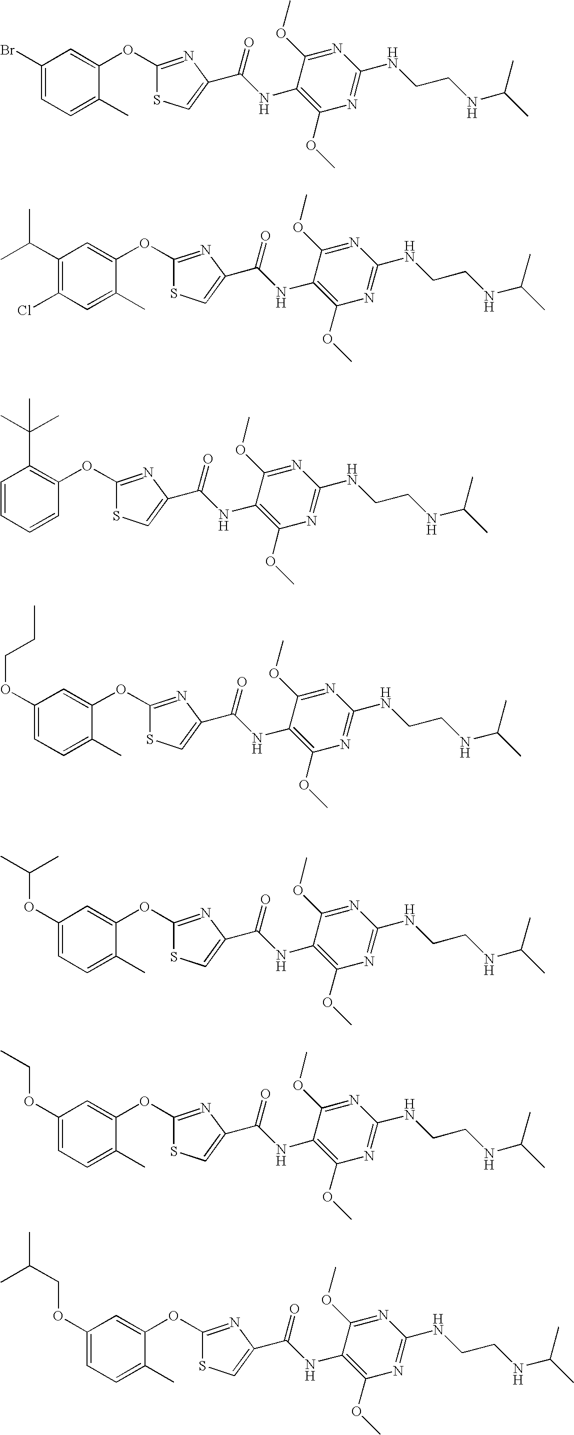

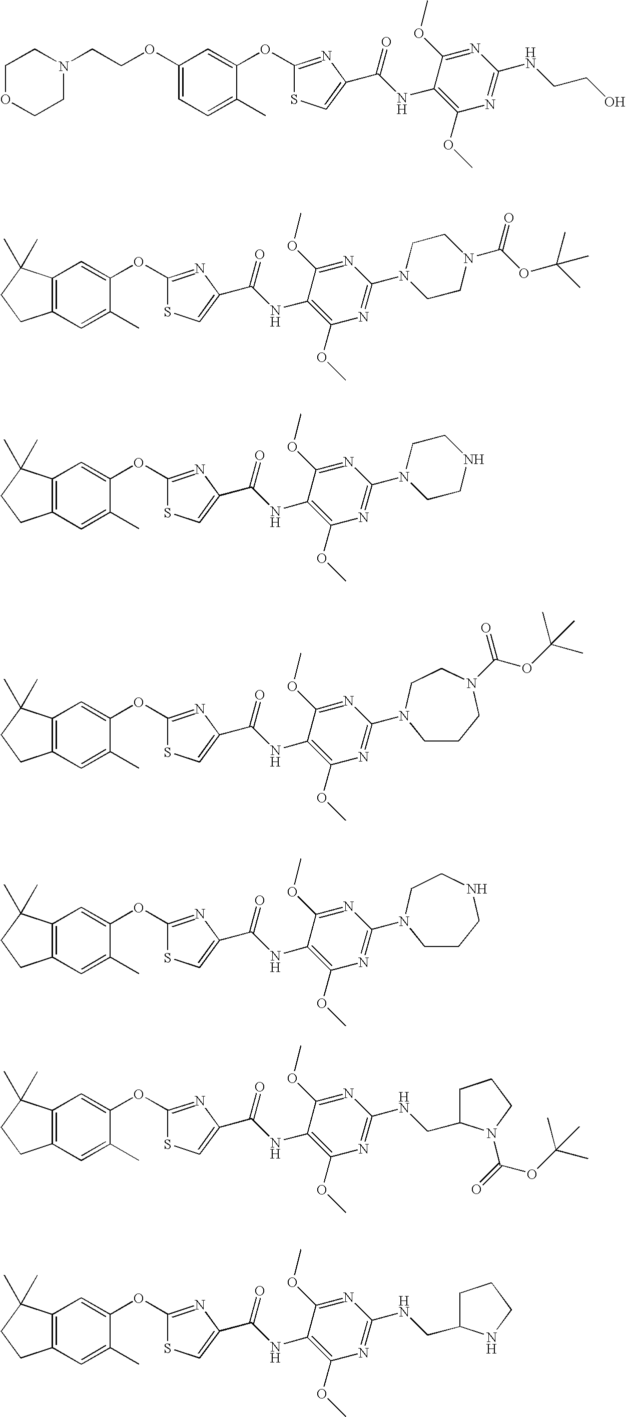

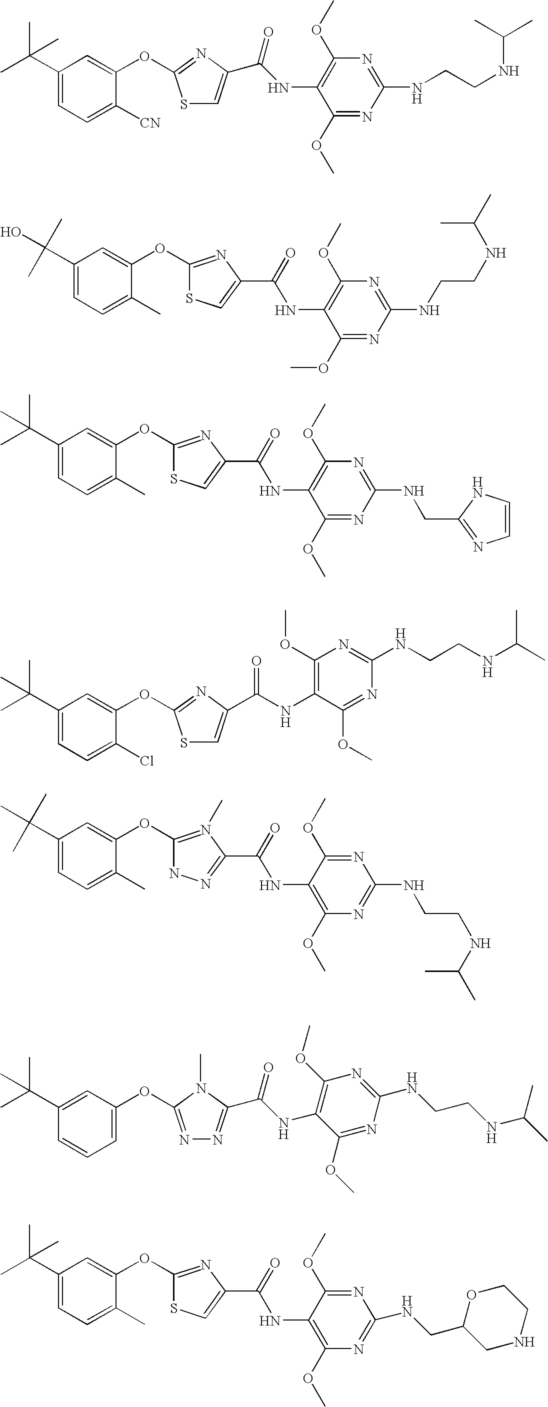

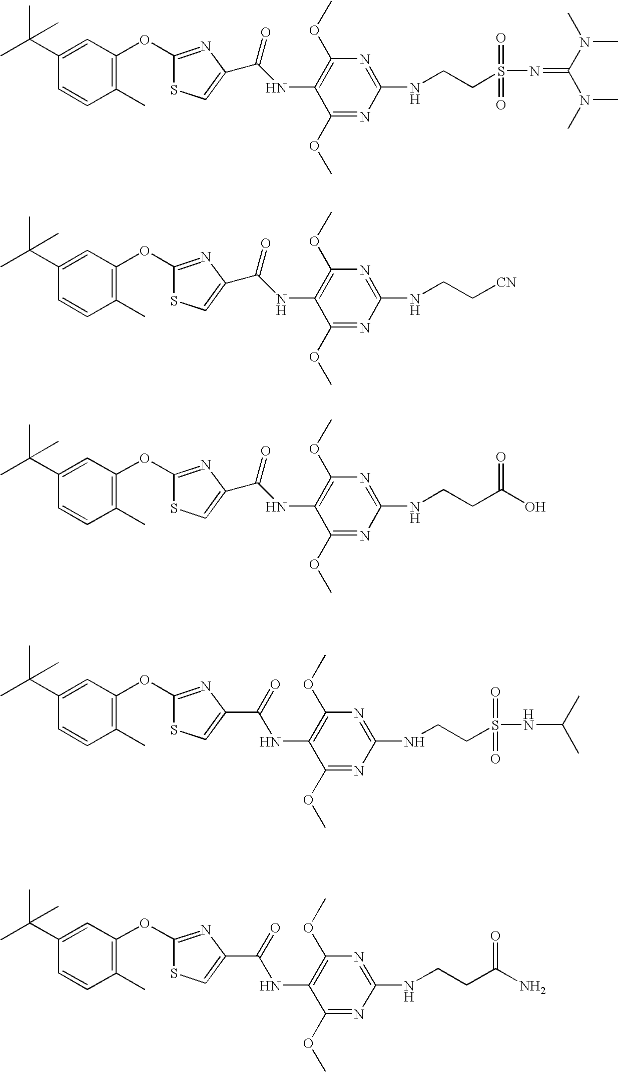

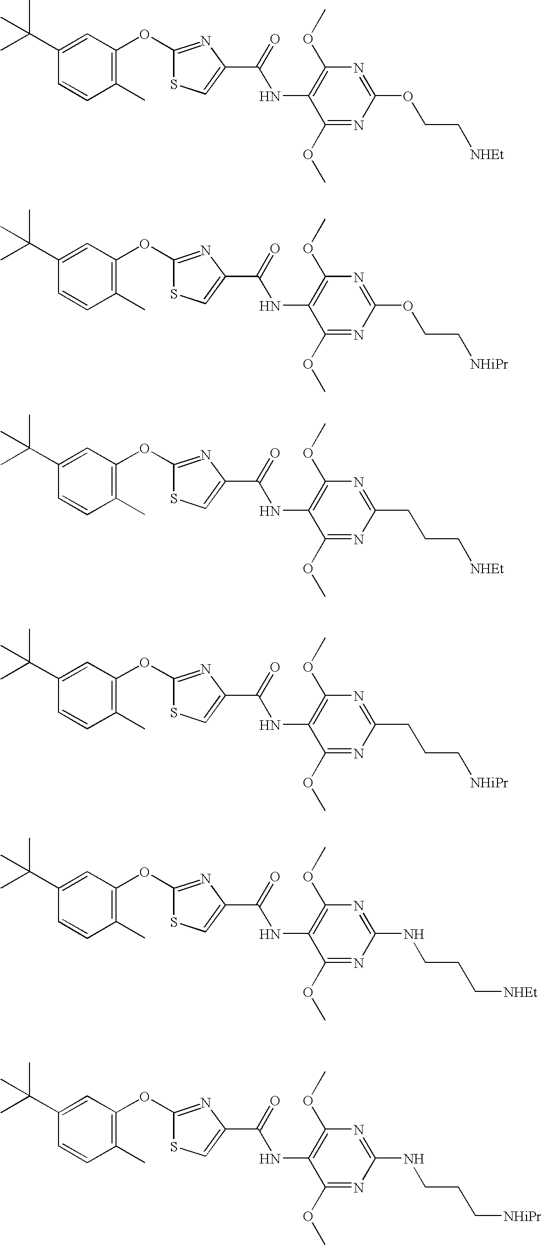

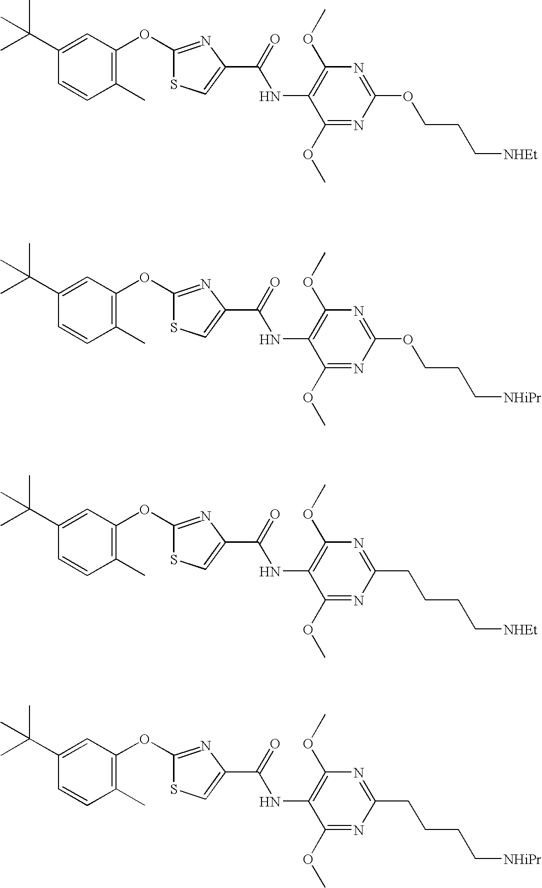

These compounds may be named as follows:
2-(5-tert-butyl-2-methylphenoxy)-N-(2-(2-(dimethylamino)ethylamino)-4,6-dimethoxypyrimidin-5-yl)oxazole-4-carboxamide
2-(5-tert-butyl-2-methylphenoxy)-N-(2-(2-(dimethylamino)ethylamino)-4,6-dimethoxypyrimidin-5-yl)thiazole-4-carboxamide
2-(5-tert-butyl-2-methylphenoxy)-N-(2-(dimethylamino)-4,6-dimethoxypyrimidin-5-yl)oxazole-4-carboxamide
2-(5-tert-butyl-2-methylphenoxy)-N-(2-(dimethylamino)-4,6-dimethoxypyrimidin-5-yl)thiazole-4-carboxamide
2-(5-tert-butyl-2-methylphenoxy)-N-(4,6-dimethoxy-2-(1-methylazetidin-3-ylamino)pyrimidin-5-yl)oxazole-4-carboxamide
2-(5-tert-butyl-2-methylphenoxy)-N-(4,6-dimethoxy-2-(1-methylazetidin-3-ylamino)pyrimidin-5-yl)thiazole-4-carboxamide
2-(3-tert-butylphenoxy)-N-(2-(2-(dimethylamino)ethylamino)-4,6-dimethoxypyrimidin-5-yl)oxazole-4-carboxamide
2-(3-tert-butylphenoxy)-N-(2-(2-(dimethylamino)ethylamino)-4,6-dimethoxypyrimidin-5-yl)thiazole-4-carboxamide
2-(3-tert-butylphenoxy)-N-(2-(dimethylamino)-4,6-dimethoxypyrimidin-5-yl)oxazole-4-carboxamide
2-(3-tert-butylphenoxy)-N-(2-(dimethylamino)-4,6-dimethoxypyrimidin-5-yl)thiazole-4-carboxamide
2-(3-tert-butylphenoxy)-N-(4,6-dimethoxy-2-(1-methylazetidin-3-ylamino)pyrimidin-5-yl)oxazole-4-carboxamide
2-(3-tert-butylphenoxy)-N-(4,6-dimethoxy-2-(1-methylazetidin-3-ylamino)pyrimidin-5-yl)thiazole-4-carboxamide
2-(3-tert-butylphenoxy)-N-(4,6-dimethoxy-2-(3-morpholinopropylamino)pyrimidin-5-yl)oxazole-4-carboxamide
2-(3-tert-butylphenoxy)-N-(4,6-dimethoxy-2-(3-morpholinopropylamino)pyrimidin-5-yl)thiazole-4-carboxamide
2-(3,3-dimethyl-2,3-dihydro-1H-inden-5-yloxy)-N-(2-(2-(dimethylamino)ethylamino)-4,6-dimethoxypyrimidin-5-yl)oxazole-4-carboxamide
2-(3,3-dimethyl-2,3-dihydro-1H-inden-5-yloxy)-N-(2-(2-(dimethylamino)ethylamino)-4,6-dimethoxypyrimidin-5-yl)thiazole-4-carboxamide
2-(3,3-dimethyl-2,3-dihydro-1H-inden-5-yloxy)-N-(2-(dimethylamino)-4,6-dimethoxypyrimidin-5-yl)oxazole-4-carboxamide
2-(3,3-dimethyl-2,3-dihydro-1H-inden-5-yloxy)-N-(2-(dimethylamino)-4,6-dimethoxypyrimidin-5-yl)thiazole-4-carboxamide
1-(4,6-dimethoxy-2-(1-methylazetidin-3-ylamino)pyrimidin-5-yl)-2-(3,3-dimethyl-2,3-dihydro-1H-inden-5-yloxy)oxazole-4-carboxamide
N-(4,6-dimethoxy-2-(1-methylazetidin-3-ylamino)pyrimidin-5-yl)-2-(3,3-dimethyl-2,3-dihydro-1H-inden-5-yloxy)thiazole-4-carboxamide
N-(2-(2-(dimethylamino)ethylamino)-4,6-dimethoxypyrimidin-5-yl)-2-(3,3,6-trimethyl-2,3-dihydro-1H-inden-5-yloxy)oxazole-4-carboxamide
N-(2-(2-(dimethylamino)ethylamino)-4,6-dimethoxypyrimidin-5-yl)-2-(3,3,6-trimethyl-2,3-dihydro-1H-inden-5-yloxy)thiazole-4-carboxamide
N-(2-(dimethylamino)-4,6-dimethoxypyrimidin-5-yl)-2-(3,3,6-trimethyl-2,3-dihydro-1H-inden-5-yloxy)oxazole-4-carboxamide
N-(2-(dimethylamino)-4,6-dimethoxypyrimidin-5-yl)-2-(3,3,6-trimethyl-2,3-dihydro-1H-inden-5-yloxy)thiazole-4-carboxamide
N-(4,6-dimethoxy-2-(1-methylazetidin-3-ylamino)pyrimidin-5-yl)-2-(3,3,6-trimethyl-2,3-dihydro-1H-inden-5-yloxy)oxazole-4-carboxamide
N-(4,6-dimethoxy-2-(1-methylazetidin-3-ylamino)pyrimidin-5-yl)-2-(3,3,6-trimethyl-2,3-dihydro-1H-inden-5-yloxy)thiazole-4-carboxamide
2-(5-tert-butyl-2-methylphenoxy)-N-(4,6-dimethoxy-2-(methylamino)pyrimidin-5-yl)oxazole-4-carboxamide
2-(5-tert-butyl-2-methylphenoxy)-N-(2-(3-(dimethylamino)propylamino)-4,6-dimethoxypyrimidin-5-yl)oxazole-4-carboxamide
N-(2-(2-aminoethylamino)-4,6-dimethoxypyrimidin-5-yl)-2-(5-tert-butyl-2-methylphenoxy)oxazole-4-carboxamide
2-(5-tert-butyl-2-methylphenoxy)-N-(4,6-dimethoxy-2-(2-(methylamino)ethylamino)pyrimidin-5-yl)oxazole-4-carboxamide
2-(5-tert-butyl-2-methylphenoxy)-N-(2-(2-(ethyl(methyl)amino)ethylamino)-4,6-dimethoxypyrimidin-5-yl)oxazole-4-carboxamide
N-(2-(2-(azetidin-1-yl)ethylamino)-4,6-dimethoxypyrimidin-5-yl)-2-(5-tert-butyl-2-methylphenoxy)oxazole-4-carboxamide
2-(5-tert-butyl-2-methylphenoxy)-N-(4,6-dimethoxy-2-(2-(pyrrolidin-1-yl)ethylamino)pyrimidin-5-yl)oxazole-4-carboxamide
2-(5-tert-butyl-2-methylphenoxy)-N-(4,6-dimethoxy-2-(3-(pyrrolidin-1-yl)propylamino)pyrimidin-5-yl)oxazole-4-carboxamide
2-(5-tert-butyl-2-methylphenoxy)-N-(2-((2-(dimethylamino)ethyl)(methyl)amino)-4,6-dimethoxypyrimidin-5-yl)oxazole-4-carboxamide
2-(5-tert-butyl-2-methylphenoxy)-N-(2-((2-(trimethylammonium)ethyl)amino)-4,6-dimethoxypyrimidin-5-yl)oxazole-4-carboxamide iodide
2-(5-tert-butyl-2-methylphenoxy)−/−(4,6-dimethoxy-2-(1-methylpiperidin-4-ylamino)pyrimidin-5-yl)oxazole-4-carboxamide
2-(5-tert-butyl-2-methylphenoxy)-N-(4,6-dimethoxy-2-(piperidin-4-ylamino)pyrimidin-5-yl)oxazole-4-carboxamide
2-(5-tert-butyl-2-methylphenoxy)-N-(4,6-dimethoxy-2-(1-methylpiperidin-4-yloxy)pyrimidin-5-yl)oxazole-4-carboxamide
2-(5-tert-butyl-2-methylphenoxy)-N-(4,6-dimethoxy-2-(piperidin-4-yloxy)pyrimidin-5-yl)oxazole-4-carboxamide
N-(2-(azetidin-3-ylamino)-4,6-dimethoxypyrimidin-5-yl)-2-(5-tert-butyl-2-methylphenoxy)oxazole-4-carboxamide
2-(5-tert-butyl-2-methylphenoxy)-N-(4,6-dimethoxy-2-(1-methylazetidin-3-yloxy)pyrimidin-5-yl)oxazole-4-carboxamide
N-(2-(azetidin-3-yloxy)-4,6-dimethoxypyrimidin-5-yl)-2-(5-tert-butyl-2-methylphenoxy)oxazole-4-carboxamide
N-(2-(3-aminopyrrolidin-1-yl)-4,6-dimethoxypyrimidin-5-yl)-2-(5-tert-butyl-2-methylphenoxy)oxazole-4-carboxamide
2-(5-tert-butyl-2-methylphenoxy)-N-(2-((1-ethylpyrrolidin-2-yl)methylamino)-4,6-dimethoxypyrimidin-5-yl)oxazole-4-carboxamide
2-(5-tert-butyl-2-methylphenoxy)−/−(2-((pyrrolidin-2-yl)methylamino)-4,6-dimethoxypyrimidin-5-yl)oxazole-4-carboxamide
2-(5-tert-butyl-2-methylphenoxy)-N-(2-(2-hydroxyethylamino)-4,6-dimethoxypyrimidin-5-yl)oxazole-4-carboxamide
2-(5-tert-butyl-2-methylphenoxy)-N-(2-(2-hydroxyethylamino)-4,6-dimethoxypyrimidin-5-yl)thiazole-4-carboxamide
N-(2-(2-aminoethylamino)-4,6-dimethoxypyrimidin-5-yl)-2-(5-tert-butyl-2-methylphenoxy)thiazole-4-carboxamide
2-(5-tert-butyl-2-methylphenoxy)-N-(4,6-dimethoxy-2-(2-(methylamino)ethylamino)pyrimidin-5-yl)thiazole-4-carboxamide
2-(5-tert-butyl-2-methylphenoxy)-N-(2-(2-(ethylamino)ethylamino)-4,6-dimethoxypyrimidin-5-yl)oxazole-4-carboxamide
2-(5-tert-butyl-2-methylphenoxy)-N-(2-(2-(ethylamino)ethylamino)-4,6-dimethoxypyrimidin-5-yl)thiazole-4-carboxamide
2-(5-tert-butyl-2-methylphenoxy)-N-(2-(2-(isopropylamino)ethylamino)-4,6-dimethoxypyrimidin-5-yl)oxazole-4-carboxamide
2-(5-tert-butyl-2-methylphenoxy)-N-(2-(2-(isopropylamino)ethylamino)-4,6-dimethoxypyrimidin-5-yl)thiazole-4-carboxamide
2-(5-tert-butyl-2-methylphenoxy)-N-(2-(2-(tert-butylamino)ethylamino)-4,6-dimethoxypyrimidin-5-yl)oxazole-4-carboxamide
2-(5-tert-butyl-2-methylphenoxy)-N-(2-(2-(tert-butylamino)ethylamino)-4,6-dimethoxypyrimidin-5-yl)thiazole-4-carboxamide
N-(2-(2-hydroxyethylamino)-4,6-dimethoxypyrimidin-5-yl)-2-(5-isopropyl-2-methylphenoxy)oxazole-4-carboxamide
N-(2-(2-hydroxyethylamino)-4,6-dimethoxypyrimidin-5-yl)-2-(5-isopropyl-2-methylphenoxy)thiazole-4-carboxamide
N-(2-(2-aminoethylamino)-4,6-dimethoxypyrimidin-5-yl)-2-(5-isopropyl-2-methylphenoxy)oxazole-4-carboxamide
N-(2-(2-aminoethylamino)-4,6-dimethoxypyrimidin-5-yl)-2-(5-isopropyl-2-methylphenoxy)thiazole-4-carboxamide
N-(4,6-dimethoxy-2-(2-(methylamino)ethylamino)pyrimidin-5-yl)-2-(5-isopropyl-2-methylphenoxy)oxazole-4-carboxamide
N-(4,6-dimethoxy-2-(2-(methylamino)ethylamino)pyrimidin-5-yl)-2-(5-isopropyl-2-methylphenoxy)thiazole-4-carboxamide
N-(4,6-dimethoxy-2-(2-(ethylamino)ethylamino)pyrimidin-5-yl)-2-(5-isopropyl-2-methylphenoxy)oxazole-4-carboxamide
N-(4,6-dimethoxy-2-(2-(ethylamino)ethylamino)pyrimidin-5-yl)-2-(5-isopropyl-2-methylphenoxy)thiazole-4-carboxamide
2-(5-isopropyl-2-methylphenoxy)-N-(2-(2-(isopropylamino)ethylamino)-4,6-dimethoxypyrimidin-5-yl)oxazole-4-carboxamide
2-(5-isopropyl-2-methylphenoxy)-N-(2-(2-(isopropylamino)ethylamino)-4,6-dimethoxypyrimidin-5-yl)thiazole-4-carboxamide
N-(2-(2-(tert-butylamino)ethylamino)-4,6-dimethoxypyrimidin-5-yl)-2-(5-isopropyl-2-methylphenoxy)oxazole-4-carboxamide
N-(2-(2-(tert-butylamino)ethylamino)-4,6-dimethoxypyrimidin-5-yl)-2-(5-isopropyl-2-methylphenoxy)thiazole-4-carboxamide
N-(2-(2-hydroxyethylamino)-4,6-dimethoxypyrimidin-5-yl)-2-(3,3,6-trimethyl-2,3-dihydro-1H-inden-5-yloxy)oxazole-4-carboxamide
N-(2-(2-hydroxyethylamino)-4,6-dimethoxypyrimidin-5-yl)-2-(3,3,6-trimethyl-2,3-dihydro-1H-inden-5-yloxy)thiazole-4-carboxamide
N-(2-(2-aminoethylamino)-4,6-dimethoxypyrimidin-5-yl)-2-(3,3,6-trimethyl-2,3-dihydro-1H-inden-5-yloxy)oxazole-4-carboxamide
N-(2-(2-aminoethylamino)-4,6-dimethoxypyrimidin-5-yl)-2-(3,3,6-trimethyl-2,3-dihydro-1H-inden-5-yloxy)thiazole-4-carboxamide
N-(4,6-dimethoxy-2-(2-(methylamino)ethylamino)pyrimidin-5-yl)-2-(3,3,6-trimethyl-2,3-dihydro-1H-inden-5-yloxy)oxazole-4-carboxamide
N-(4,6-dimethoxy-2-(2-(methylamino)ethylamino)pyrimidin-5-yl)-2-(3,3,6-trimethyl-2,3-dihydro-1H-inden-5-yloxy)thiazole-4-carboxamide
N-(2-(2-(ethylamino)ethylamino)-4,6-dimethoxypyrimidin-5-yl)-2-(3,3,6-trimethyl-2,3-dihydro-1H-inden-5-yloxy)oxazole-4-carboxamide
N-(2-(2-(ethylamino)ethylamino)-4,6-dimethoxypyrimidin-5-yl)-2-(3,3,6-trimethyl-2,3-dihydro-1H-inden-5-yloxy)thiazole-4-carboxamide
N-(2-(2-(isopropylamino)ethylamino)-4,6-dimethoxypyrimidin-5-yl)-2-(3,3,6-trimethyl-2,3-dihydro-1H-inden-5-yloxy)oxazole-4-carboxamide
N-(2-(2-(isopropylamino)ethylamino)-4,6-dimethoxypyrimidin-5-yl)-2-(3,3,6-trimethyl-2,3-dihydro-1H-inden-5-yloxy)thiazole-4-carboxamide
N-(2-(2-(tert-butylamino)ethylamino)-4,6-dimethoxypyrimidin-5-yl)-2-(3,3,6-trimethyl-2,3-dihydro-1H-inden-5-yloxy)oxazole-4-carboxamide
N-(2-(2-(tert-butylamino)ethylamino)-4,6-dimethoxypyrimidin-5-yl)-2-(3,3,6-trimethyl-2,3-dihydro-1H-inden-5-yloxy)thiazole-4-carboxamide
2-(5-tert-butyl-2-methylphenoxy)-N-(4,6-dimethoxy-2-(2-oxoimidazolidin-1-yl)pyrimidin-5-yl)oxazole-4-carboxamide
2-(5-tert-butyl-2-methylphenoxy)-N-(4,6-dimethoxy-2-(2-oxoimidazolidin-1-yl)pyrimidin-5-yl)thiazole-4-carboxamide
N-(2-(2-tert-Butoxyethoxy)-4,6-dimethoxypyrimidin-5-yl)-2-(5-tert-butyl-2-methylphenoxy)thiazole-4-carboxamide
2-(5-tert-Butyl-2-methylphenoxy)-N-(2-(2-hydroxyethoxy)-4,6-dimethoxypyrimidin-5-yl)thiazole-4-carboxamide
2-(5-Chloro-2-methylphenoxy)-N-(2-(2-hydroxyethylamino)-4,6-dimethoxypyrimidin-5-yl)thiazole-4-carboxamide
2-(2,5-Dimethylphenoxy)-N-(2-(2-hydroxyethylamino)-4,6-dimethoxypyrimidin-5-yl)thiazole-4-carboxamide
N-(2-(2-Hydroxyethylamino)-4,6-dimethoxypyrimidin-5-yl)-2-(o-tolyloxy)thiazole-4-carboxamide
2-(2-Chloro-5-(trifluoromethyl)phenoxy)-N-(2-(2-hydroxyethylamino)-4,6-dimethoxypyrimidin-5-yl)thiazole-4-carboxamide
2-(6-Bromo-3,3-dimethyl-2,3-dihydro-1H-inden-5-yloxy)-N-(2-(2-(isopropylamino)ethylamino)-4,6-dimethoxypyrimidin-5-yl)thiazole-4-carboxamide
2-(6-Chloro-3,3-dimethyl-2,3-dihydro-1H-inden-5-yloxy)-N-(2-(2-(isopropylamino)ethylamino)-4,6-dimethoxypyrimidin-5-yl)thiazole-4-carboxamide
N-(2-(2-(Isopropylamino)ethylamino)-4,6-dimethoxypyrimidin-5-yl)-2-(6-methoxy-3,3-dimethyl-2,3-dihydro-1H-inden-5-yloxy)thiazole-4-carboxamide
N-(2-(2-(Isopropylamino)ethylamino)-4,6-dimethoxypyrimidin-5-yl)-2-(5-methoxy-2-methylphenoxy)thiazole-4-carboxamide
2-(2-Bromo-5-tert-butylphenoxy)-N-(2-(2-(isopropylamino)ethylamino)-4,6-dimethoxypyrimidin-5-yl)thiazole-4-carboxamide
2-(5-tert-Butyl-2-methoxyphenoxy)-N-(2-(2-(isopropylamino)ethylamino)-4,6-dimethoxypyrimidin-5-yl)thiazole-4-carboxamide
2-(5-Bromo-2-methylphenoxy)-N-(2-(2-(isopropylamino)ethylamino)-4,6-dimethoxypyrimidin-5-yl)thiazole-4-carboxamide
2-(4-Chloro-5-isopropyl-2-methylphenoxy)-N-(2-(2-(isopropylamino)ethylamino)-4,6-dimethoxypyrimidin-5-yl)thiazole-4-carboxamide
2-(2-tert-Butylphenoxy)-N-(2-(2-(isopropylamino)ethylamino)-4,6-dimethoxypyrimidin-5-yl)thiazole-4-carboxamide
N-(2-(2-(Isopropylamino)ethylamino)-4,6-dimethoxypyrimidin-5-yl)-2-(2-methyl-5-propoxyphenoxy)thiazole-4-carboxamide
2-(5-Isopropoxy-2-methylphenoxy)-N-(2-(2-(isopropylamino)ethylamino)-4,6-dimethoxypyrimidin-5-yl)thiazole-4-carboxamide
2-(5-Ethoxy-2-methylphenoxy)-N-(2-(2-(isopropylamino)ethylamino)-4,6-dimethoxypyrimidin-5-yl)thiazole-4-carboxamide
2-(5-Isobutoxy-2-methylphenoxy)-N-(2-(2-(isopropylamino)ethylamino)-4,6-dimethoxypyrimidin-5-yl)thiazole-4-carboxamide
N-(2-(2-Hydroxyethylamino)-4,6-dimethoxypyrimidin-5-yl)-2-(2-methyl-5-(2-morpholinoethoxy)phenoxy)thiazole-4-carboxamide tert-butyl 4-(4,6-dimethoxy-5-(2-(3,3,6-trimethyl-2,3-dihydro-1H-inden-5-yloxy)thiazole-4-carboxamido)pyrimidin-2-yl)piperazine-1-carboxylate
N-(4,6-dimethoxy-2-(piperazin-1-yl)pyrimidin-5-yl)-2-(3,3,6-trimethyl-2,3-dihydro-1H-inden-5-yloxy)thiazole-4-carboxamide tert-butyl 4-(4,6-dimethoxy-5-(2-(3,3,6-trimethyl-2,3-dihydro-1H-inden-5-yloxy)thiazole-4-carboxamido)pyrimidin-2-yl)-1,4-diazepane-1-carboxylate
N-(2-(1,4-diazepan-1-yl)-4,6-dimethoxypyrimidin-5-yl)-2-(3,3,6-trimethyl-2,3-dihydro-1H-inden-5-yloxy)thiazole-4-carboxamide
tert-butyl 2-((4,6-dimethoxy-5-(2-(3,3,6-trimethyl-2,3-dihydro-1H-inden-5-yloxy)thiazole-4-carboxamido)pyrimidin-2-ylamino)methyl)pyrrolidine-1-carboxylate
N-(4,6-dimethoxy-2-(pyrrolidin-2-ylmethylamino)pyrimidin-5-yl)-2-(3,3,6-trimethyl-2,3-dihydro-1H-inden-5-yloxy)thiazole-4-carboxamide
2-(5-tert-butyl-2-cyanophenoxy)-N-(2-(2-(isopropylamino)ethylamino)-4,6-dimethoxypyrimidin-5-yl)thiazole-4-carboxamide
2-(5-(2-hydroxypropan-2-yl)-2-methylphenoxy)-N-(2-(2-(isopropylamino)ethylamino)-4,6-dimethoxypyrimidin-5-yl)thiazole-4-carboxamide
N-(2-((1H-imidazol-2-yl)methylamino)-4,6-dimethoxypyrimidin-5-yl)-2-(5-tert-butyl-2-methylphenoxy)thiazole-4-carboxamide
2-(5-tert-butyl-2-chlorophenoxy)-N-(2-(2-(isopropylamino)ethylamino)-4,6-dimethoxypyrimidin-5-yl)thiazole-4-carboxamide
5-(5-tert-butyl-2-methylphenoxy)-N-(2-(2-(isopropylamino)ethylamino)-4,6-dimethoxypyrimidin-5-yl)-4-methyl-4H-1,2,4-triazole-3-carboxamide
5-(3-tert-butylphenoxy)-N-(2-(2-(isopropylamino)ethylamino)-4,6-dimethoxypyrimidin-5-yl)-4-methyl-4H-1,2,4-triazole-3-carboxamide
2-(5-tert-butyl-2-methylphenoxy)-N-(4,6-dimethoxy-2-(morpholin-2-ylmethylamino)pyrimidin-5-yl)thiazole-4-carboxamide
N-(2-(2-(N-(bis(dimethylamino)methylene)sulfamoyl)ethylamino)-4,6-dimethoxypyrimidin-5-yl)-2-(5-tert-butyl-2-methylphenoxy)thiazole-4-carboxamide
2-(5-tert-butyl-2-methylphenoxy)-N-(2-(2-cyanoethylamino)-4,6-dimethoxypyrimidin-5-yl)thiazole-4-carboxamide
3-(5-(2-(5-tert-butyl-2-methylphenoxy)thiazole-4-carboxamido)-4,6-dimethoxypyrimidin-2-ylamino)propanoic acid
2-(5-tert-butyl-2-methylphenoxy)-N-(2-(2-(N-isopropylsulfamoyl)ethylamino)-4,6-dimethoxypyrimidin-5-yl)thiazole-4-carboxamide
N-(2-(3-amino-3-oxopropylamino)-4,6-dimethoxypyrimidin-5-yl)-2-(5-tert-butyl-2-methylphenoxy)thiazole-4-carboxamide
2-(5-tert-butyl-2-methylphenoxy)-N-(2-(2-(ethylamino)ethoxy)-4,6-dimethoxypyrimidin-5-yl)thiazole-4-carboxamide
2-(5-tert-butyl-2-methylphenoxy)-N-(2-(2-(isopropoylamino)ethoxy)-4,6-dimethoxypyrimidin-5-yl)thiazole-4-carboxamide
2-(5-tert-butyl-2-methylphenoxy)-N-(2-(3-(ethylamino)propyl)-4,6-dimethoxypyrimidin-5-yl)thiazole-4-carboxamide
2-(5-tert-butyl-2-methylphenoxy)-N-(2-(3-(isopropylamino)propyl)-4,6-dimethoxypyrimidin-5-yl)thiazole-4-carboxamide
2-(5-tert-butyl-2-methylphenoxy)-N-(2-(3-(ethylamino)propylamino)-4,6-dimethoxypyrimidin-5-yl)thiazole-4-carboxamide
2-(5-tert-butyl-2-methylphenoxy)-N-(2-(3-(isopropylamino)propylamino)-4,6-dimethoxypyrimidin-5-yl)thiazole-4-carboxamide
2-(5-tert-butyl-2-methylphenoxy)-N-(2-(3-(ethylamino)propoxy)-4,6-dimethoxypyrimidin-5-yl)thiazole-4-carboxamide
2-(5-tert-butyl-2-methylphenoxy)-N-(2-(3-(isopropylamino)propoxy)-4,6-dimethoxypyrimidin-5-yl)thiazole-4-carboxamide
2-(5-tert-butyl-2-methylphenoxy)-N-(2-(4-(ethylamino)butyl)-4,6-dimethoxypyrimidin-5-yl)thiazole-4-carboxamide
2-(5-tert-butyl-2-methylphenoxy)-N-(2-(4-(isopropylamino)butyl)-4,6-dimethoxypyrimidin-5-yl)thiazole-4-carboxamide

Compounds of the invention may be chiral. They may be in the form of a single enantiomer or diastereomer, or a racemate.

The compounds of the invention may be prepared in racemic form, or prepared in individual enantiomeric form by specific synthesis or resolution as will be appreciated in the art. The compounds may, for example, be resolved into their enantiomers by standard techniques, such as the formation of diastereomeric pairs by salt formation with an optically active acid followed by fractional crystallisation and regeneration of the free base. Alternatively, the enantiomers of the novel compounds may be separated by HPLC using a chiral column.

Some compounds of the formula may exist in different tautomeric forms, which also fall within the scope of the invention.

A compound of the invention may be in a protected amino, or protected hydroxy or protected carboxy form. The terms “protected amino”, “protected hydroxy” and “protected carboxy” as used herein refer to amino, hydroxy and carboxy groups which are protected in a manner familiar to those skilled in the art. For example, an amino group can be protected by a benzyloxycarbonyl, tert-butoxycarbonyl, acetyl or like group, or in the form of a phthalimido or like group. A carboxyl group can be protected in the form of a readily cleavable ester such as the methyl, ethyl, benzyl or tert-butyl ester.

Some compounds of the formula may exist in the form of solvates, for example hydrates, which also fall within the scope of the present invention.

Compounds of the invention may be in the form of pharmaceutically acceptable salts, for example, addition salts of inorganic or organic acids. Such inorganic acid addition salts include, for example, salts of hydrobromic acid, hydrochloric acid, nitric acid, phosphoric acid and sulphuric acid. Organic acid addition salts include, for example, salts of acetic acid, benzenesulphonic acid, benzoic acid, camphorsulphonic acid, citric acid, 2-(4-chlorophenoxy)-2-methylpropionic acid, 1,2-ethanedisulphonic acid, ethanesulphonic acid, ethylenediaminetetraacetic acid (EDTA), fumaric acid, glucoheptonic acid, gluconic acid, glutamic acid, glycarsamide, 4-hexylresorcinol, hippuric acid, 2-(4-hydroxybenzoyl)benzoic acid, 1-hydroxy-2-naphthoic acid, 3-hydroxy-2-naphthoic acid, 2-hydroxyethanesulphonic acid, lactobionic acid, n-dodecyl sulphuric acid, maleic acid, malic acid, mandelic acid, methanesulphonic acid, methyl sulphuric acid, mucic acid, 2-naphthalenesulphonic acid, pamoic acid, pantothenic acid, phosphanilic acid ((4-aminophenyl)phosphonic acid), picric acid, salicylic acid, stearic acid, succinic acid, tannic acid, tartaric acid, terephthalic acid, p-toluenesulphonic acid, 10-undecenoic acid and the like.

Salts of certain compounds may also be formed with inorganic bases. Such inorganic base salts include, for example, salts of aluminium, bismuth, calcium, lithium, magnesium, potassium, sodium, zinc and the like. Organic base salts include, for example, salts of N,N′-dibenzylethylenediamine, choline (as a counterion), diethanolamine, ethanolamine, ethylenediamine, N,N′-bis(dihydroabietyl)ethylenediamine, N-methylglucamine, procaine, tris(hydroxymethyl)aminomethane (“TRIS”) and the like.

It will be appreciated that such salts, provided that they are pharmaceutically acceptable, may be used in therapy. Such salts may be prepared by reacting the compound with a suitable acid in a conventional manner.

A compound of the invention may be prepared by any suitable method known in the art and/or by the following processes (in all Schemes X═O or S):

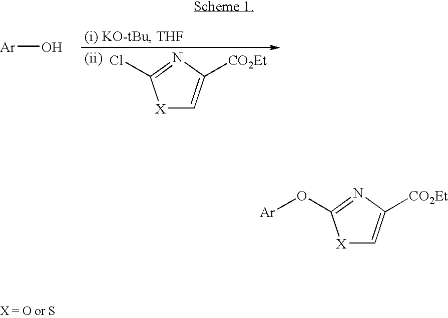

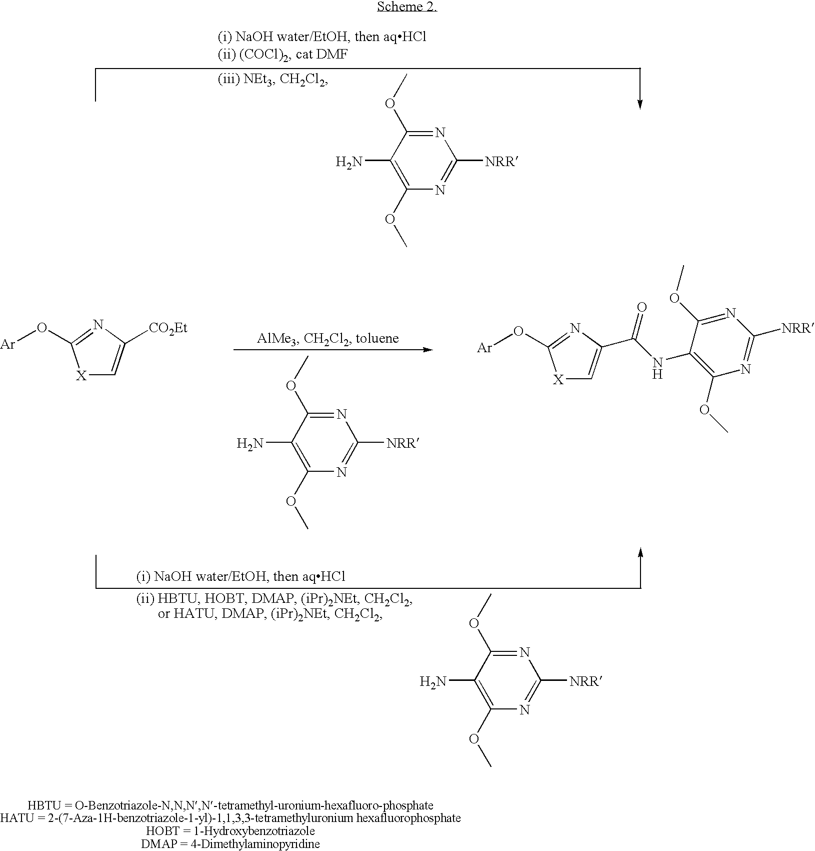

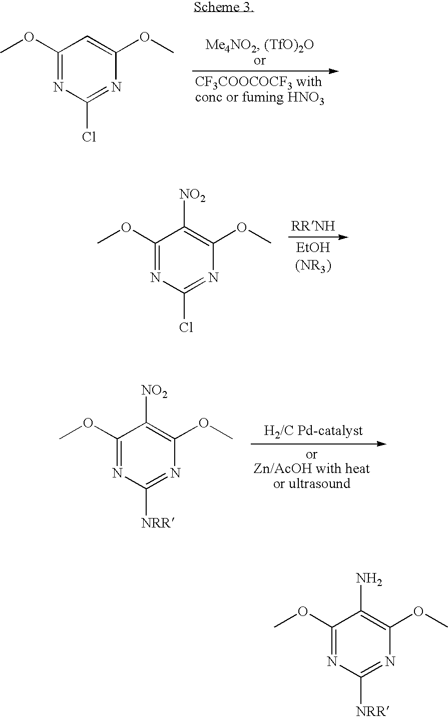

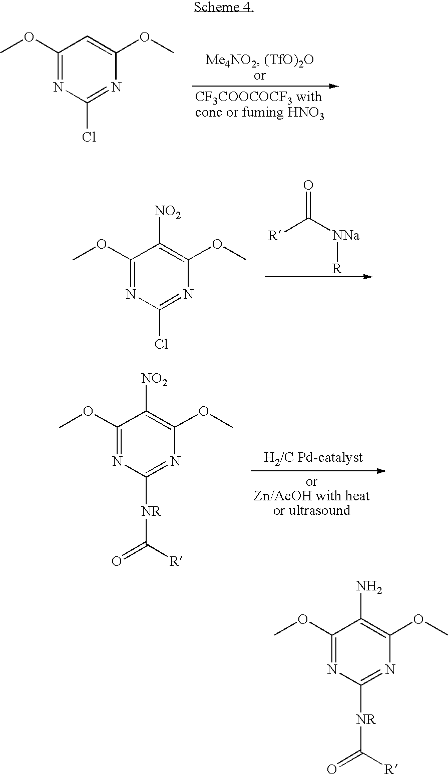

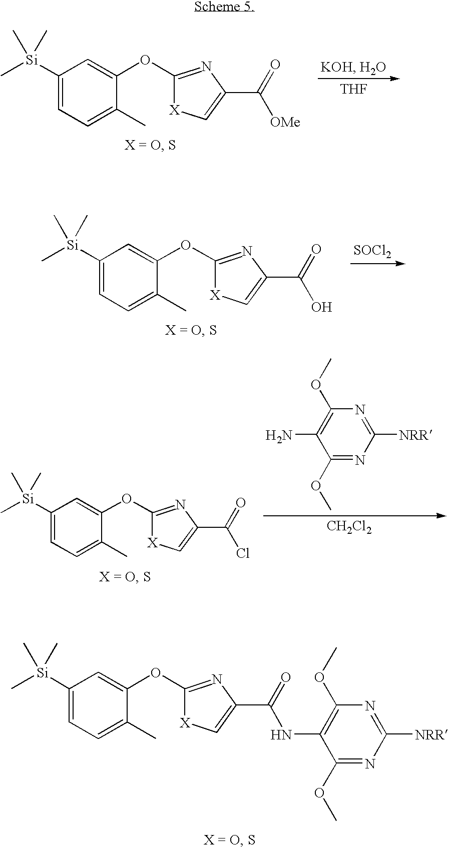

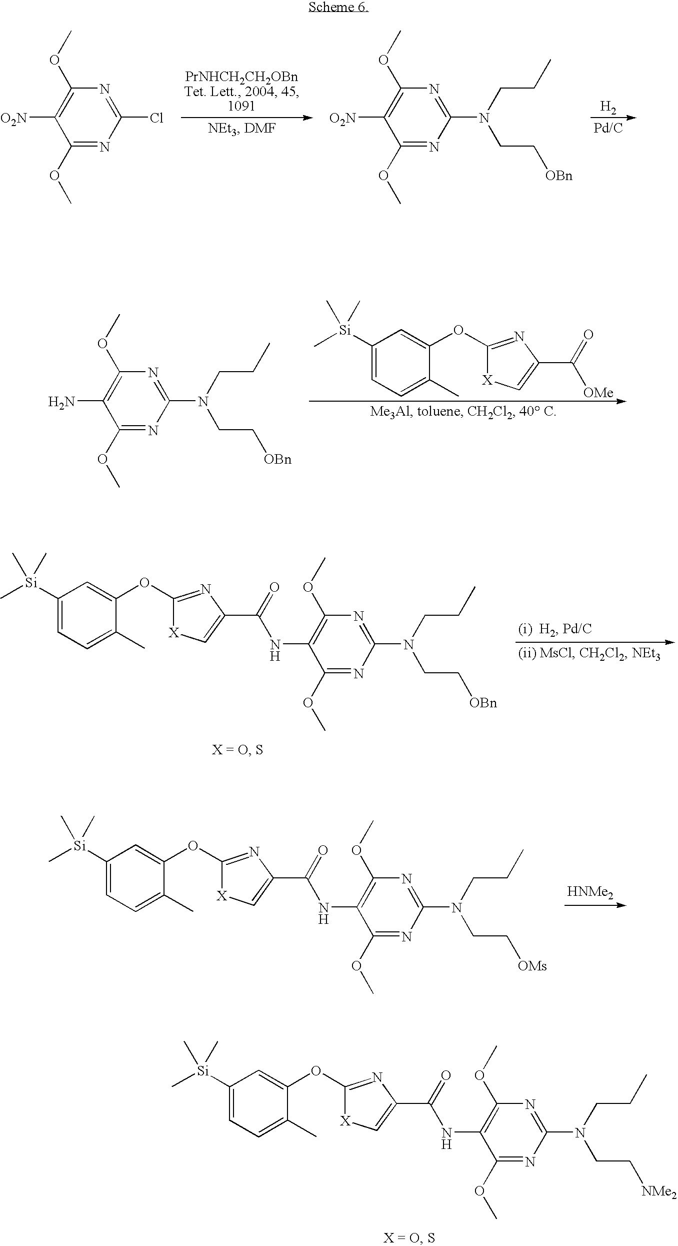

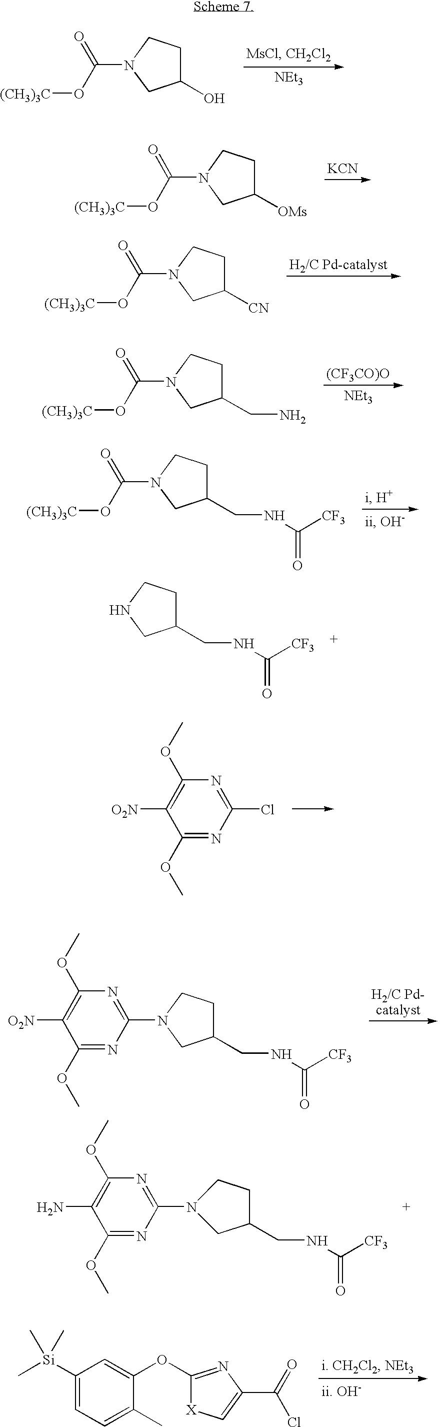

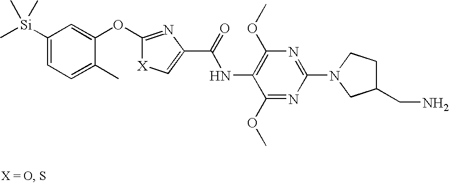

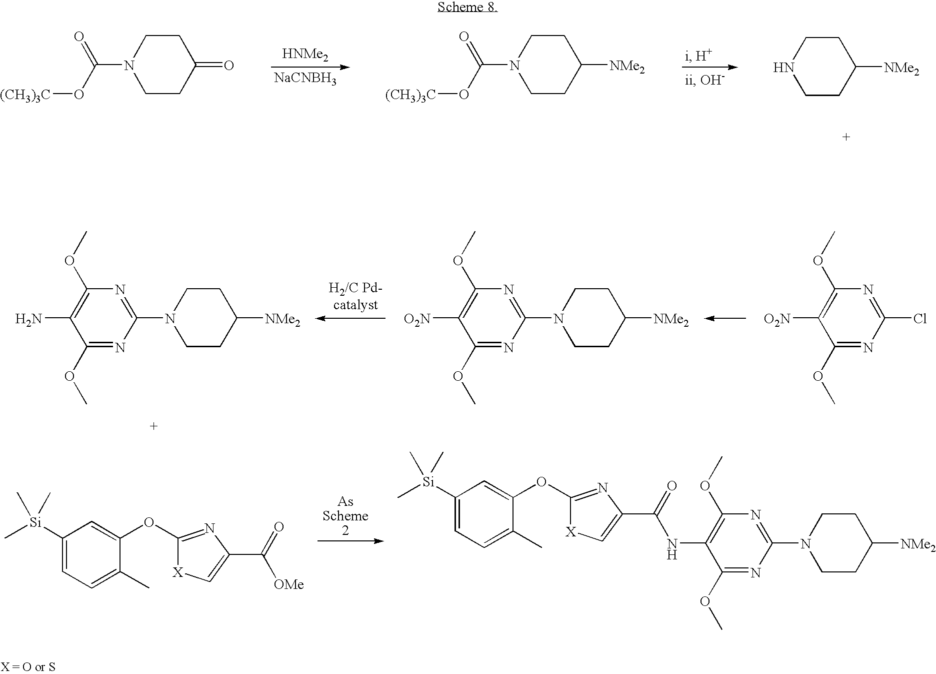

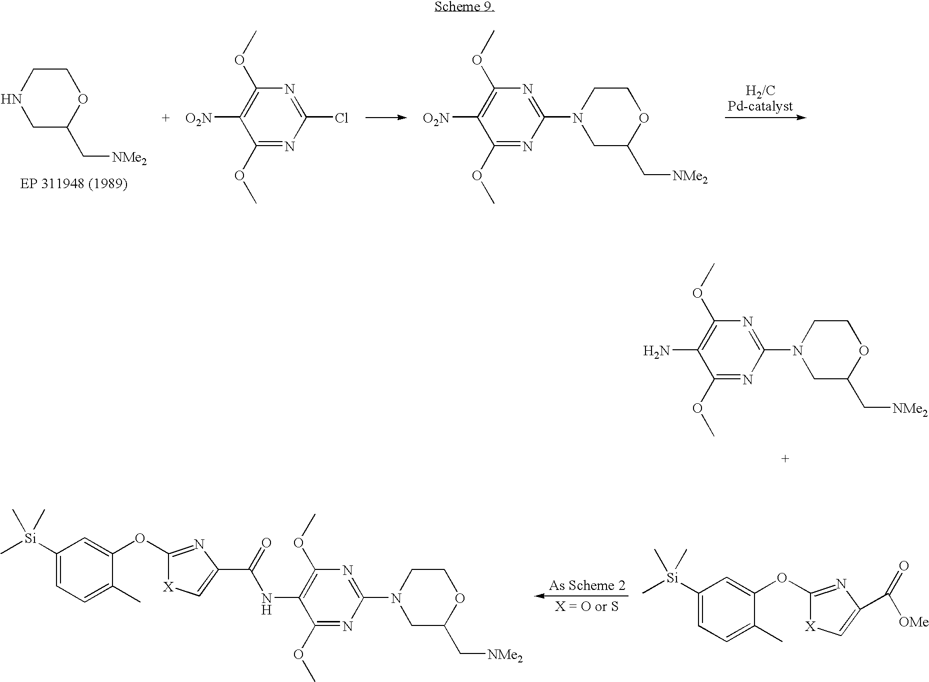

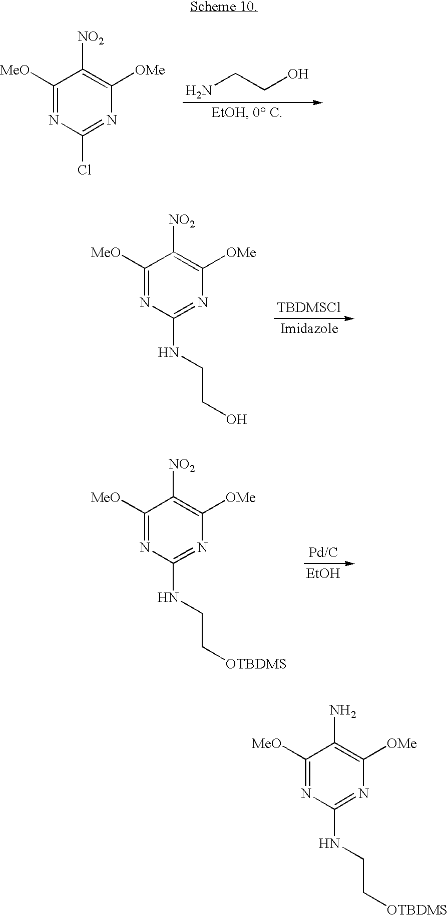

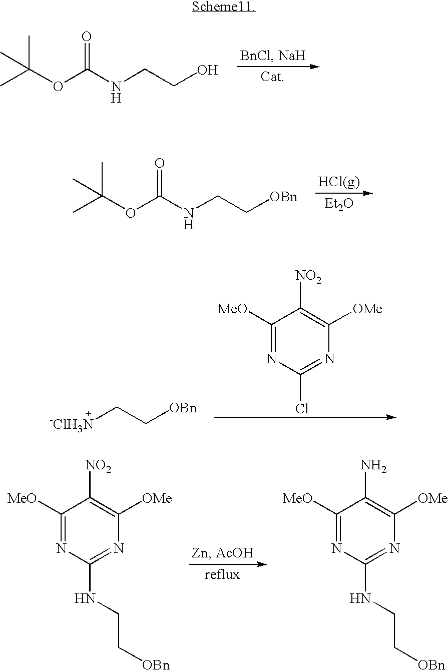

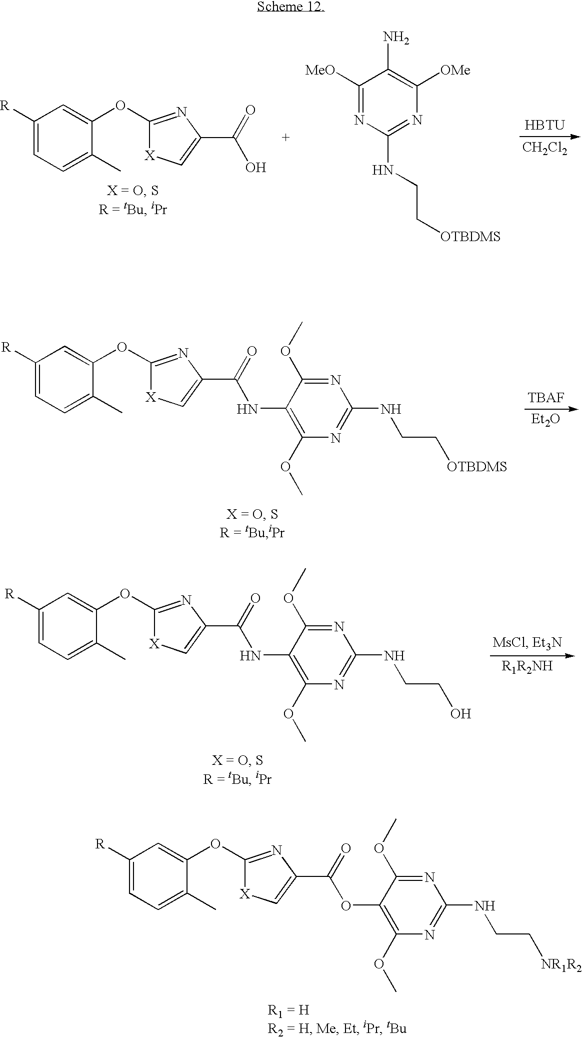

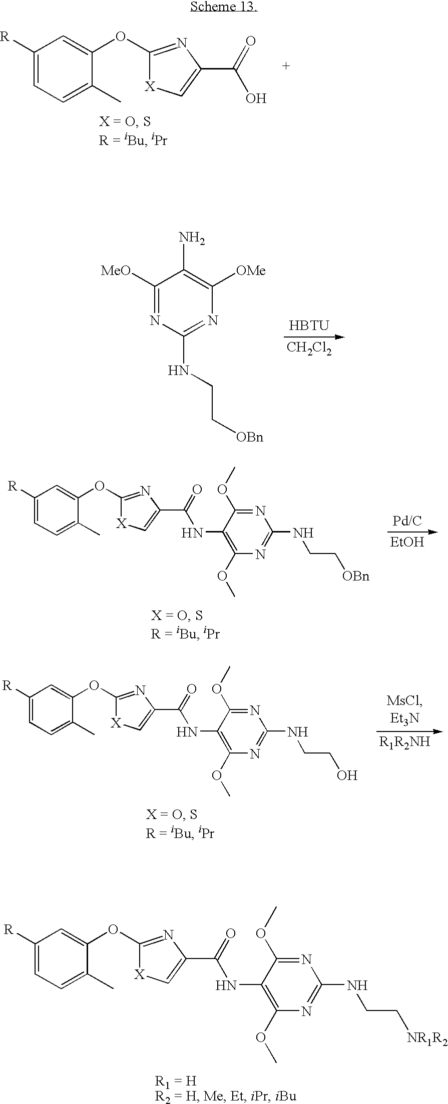

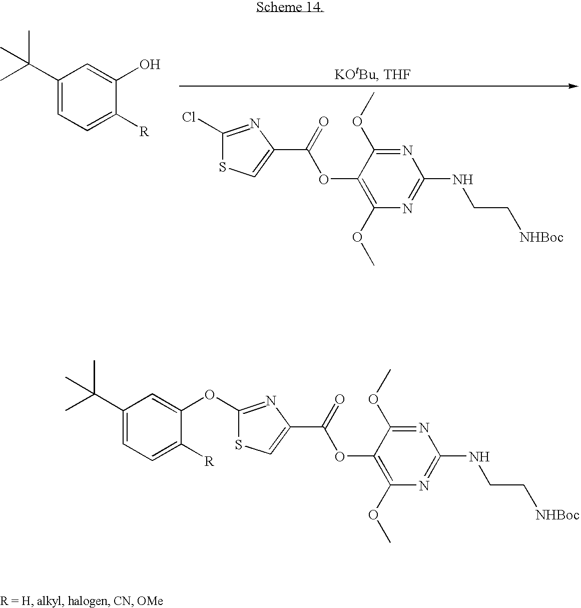

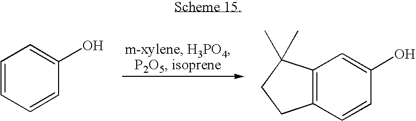

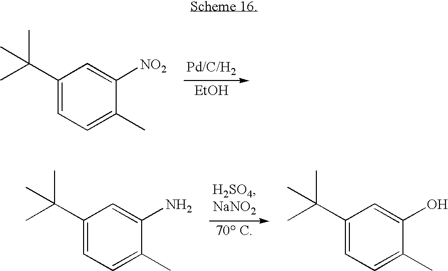

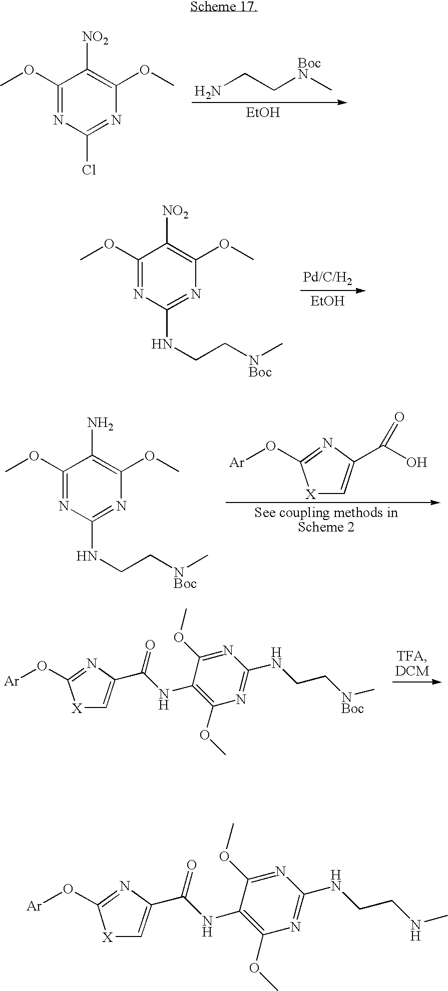

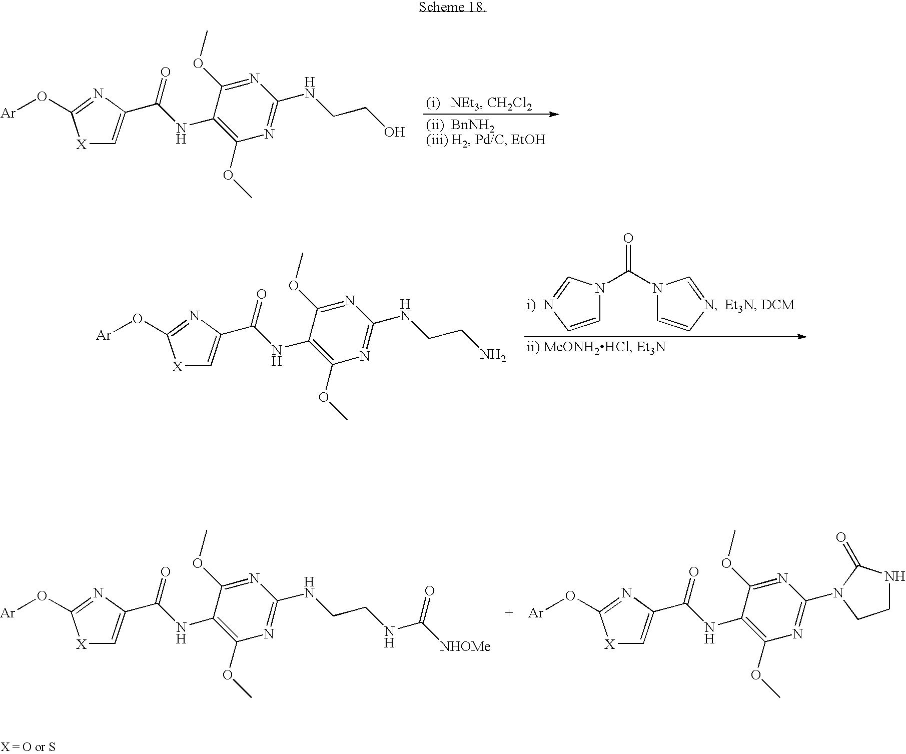

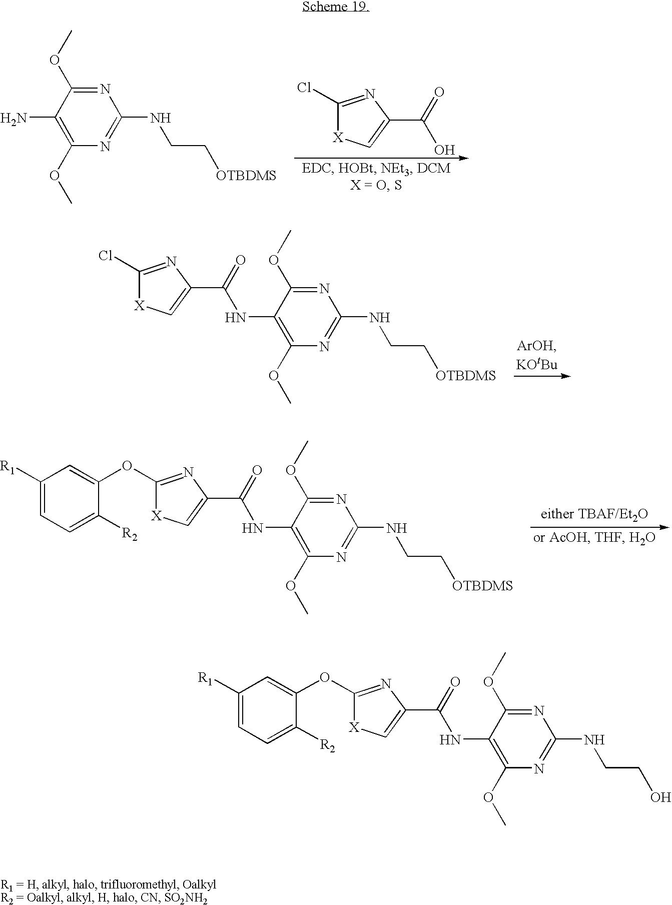

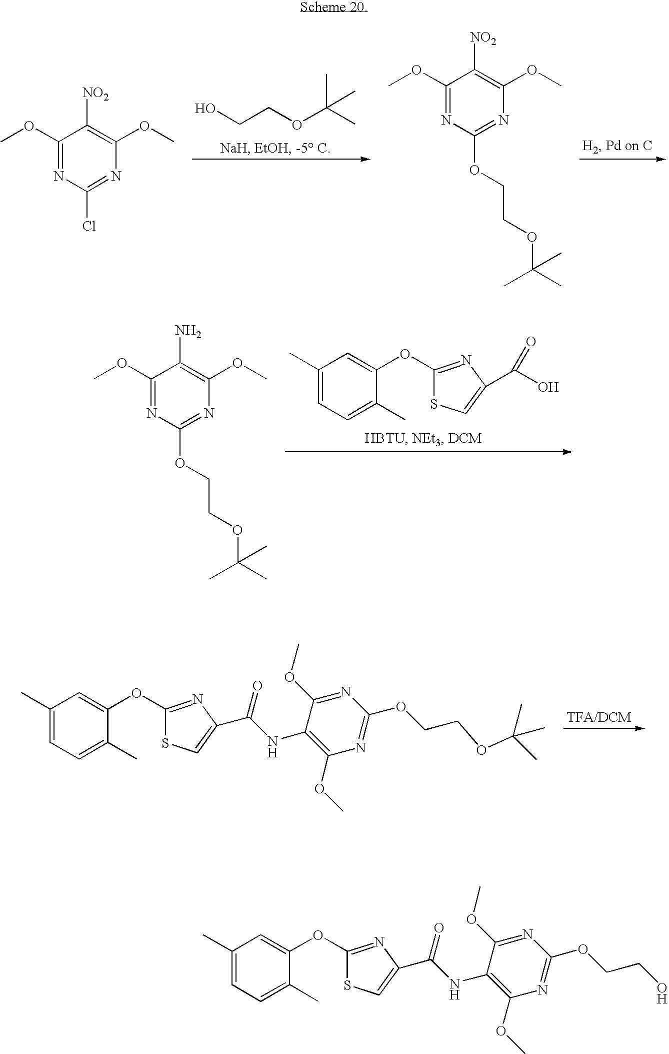

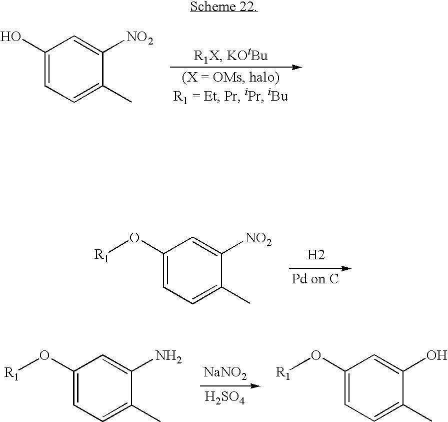

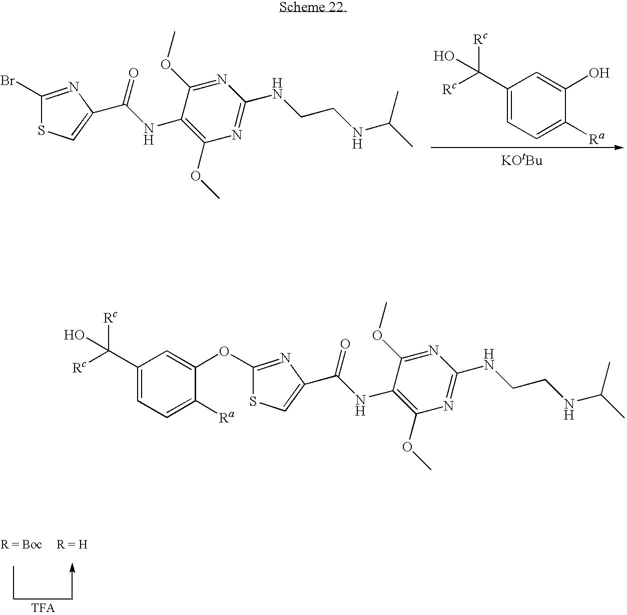

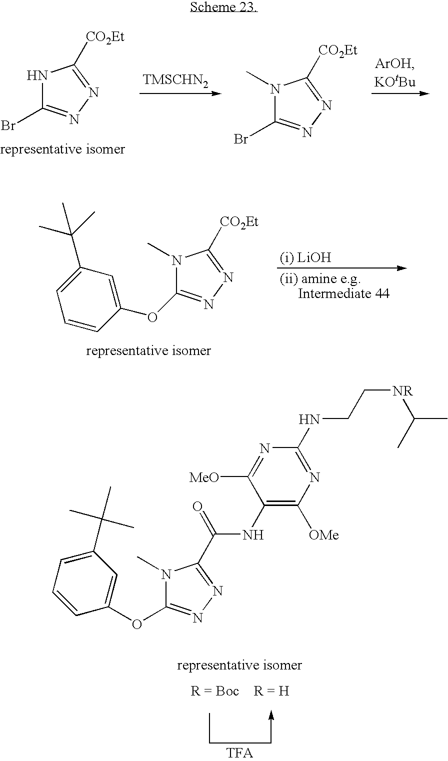

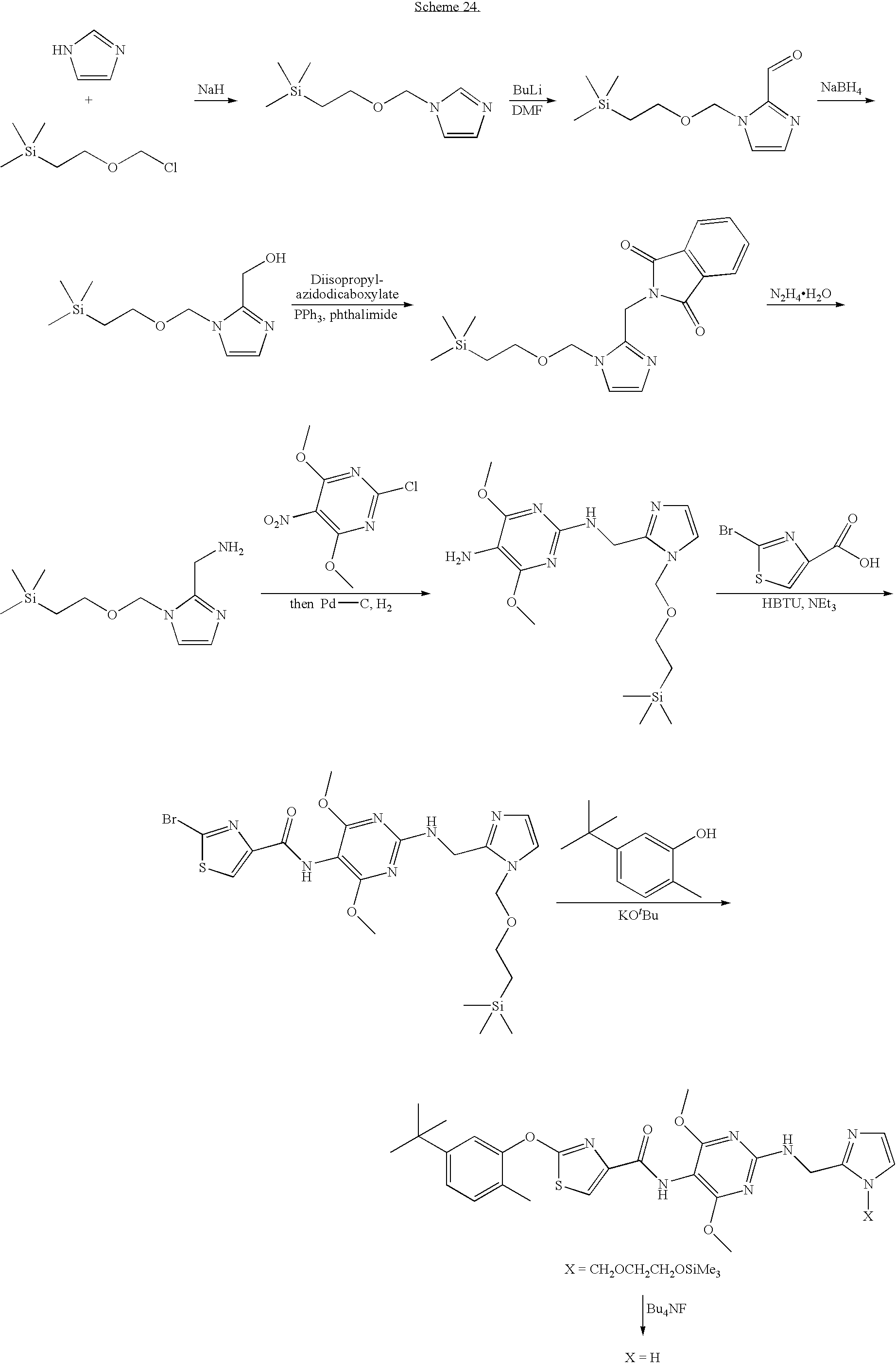

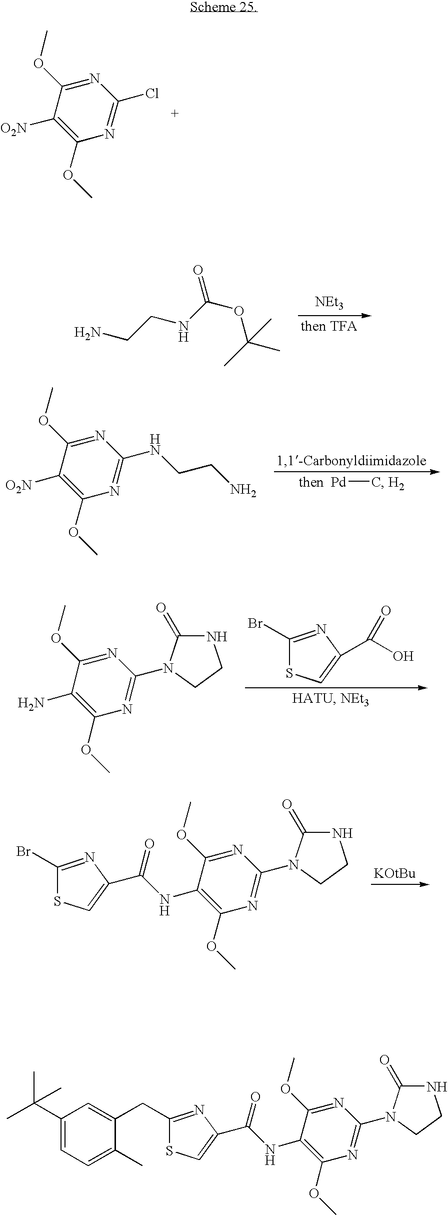

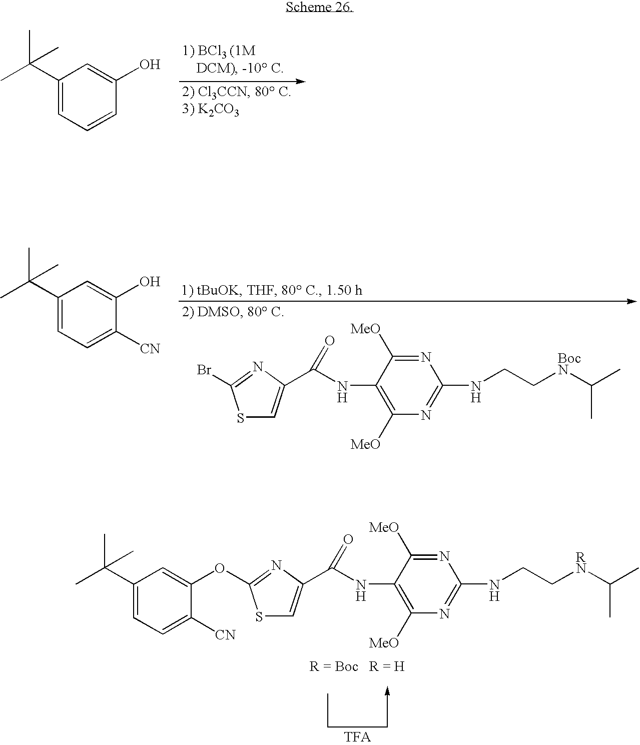

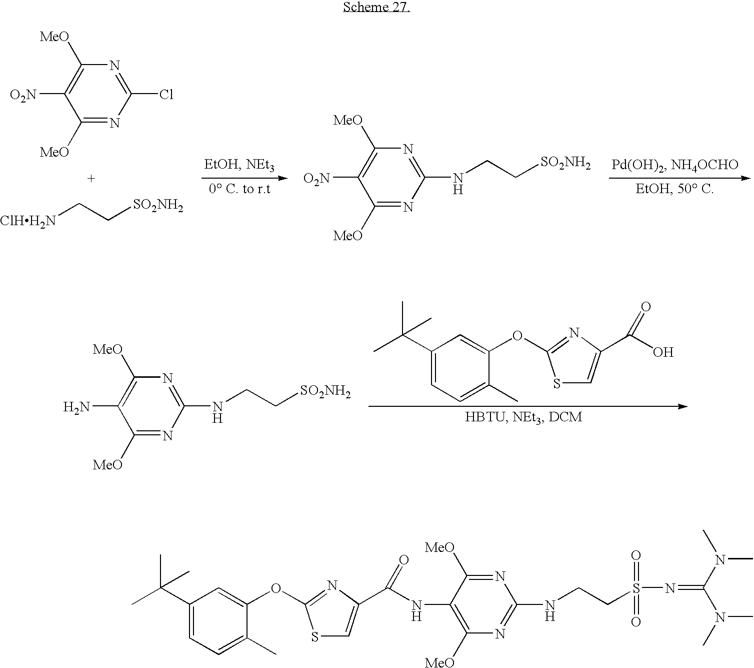

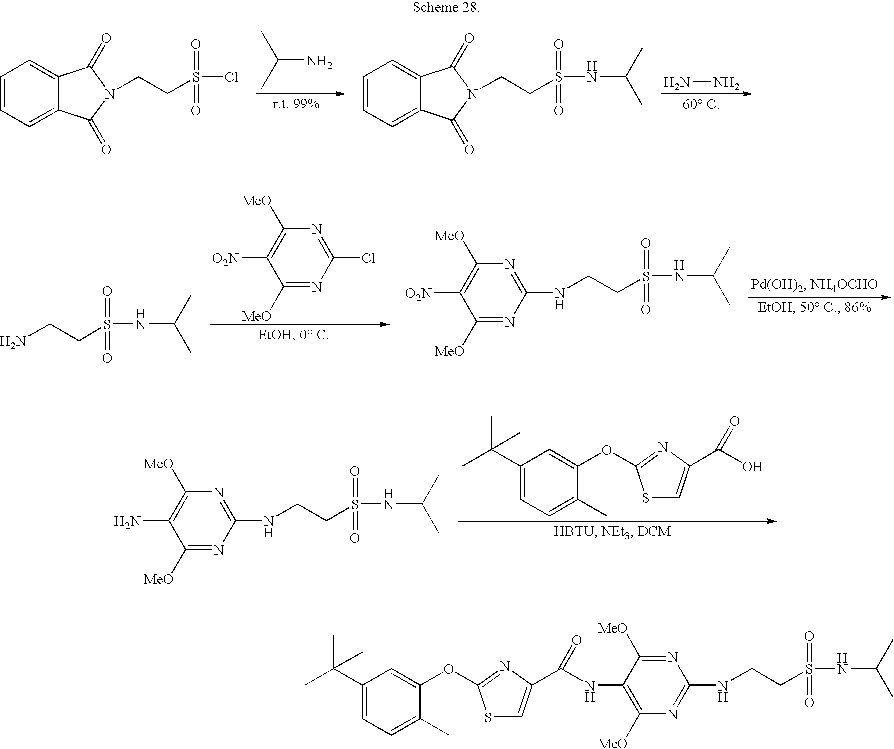

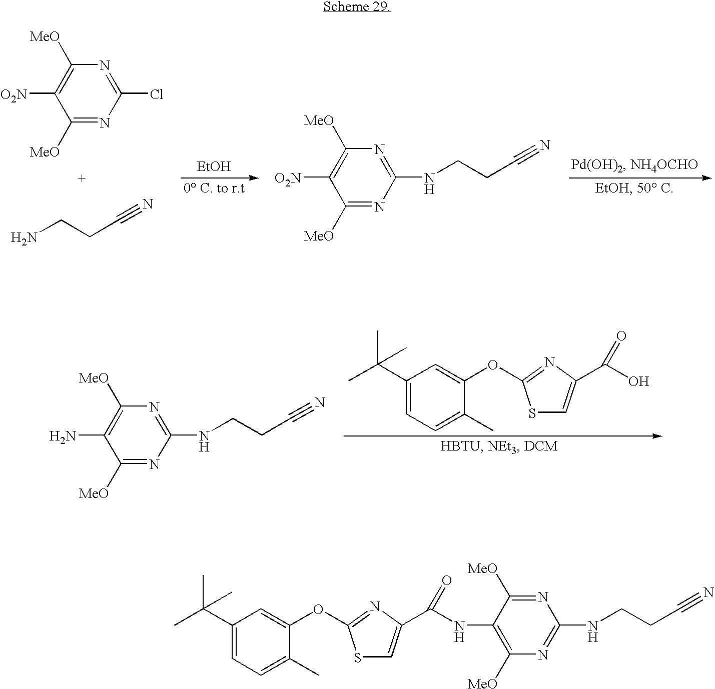

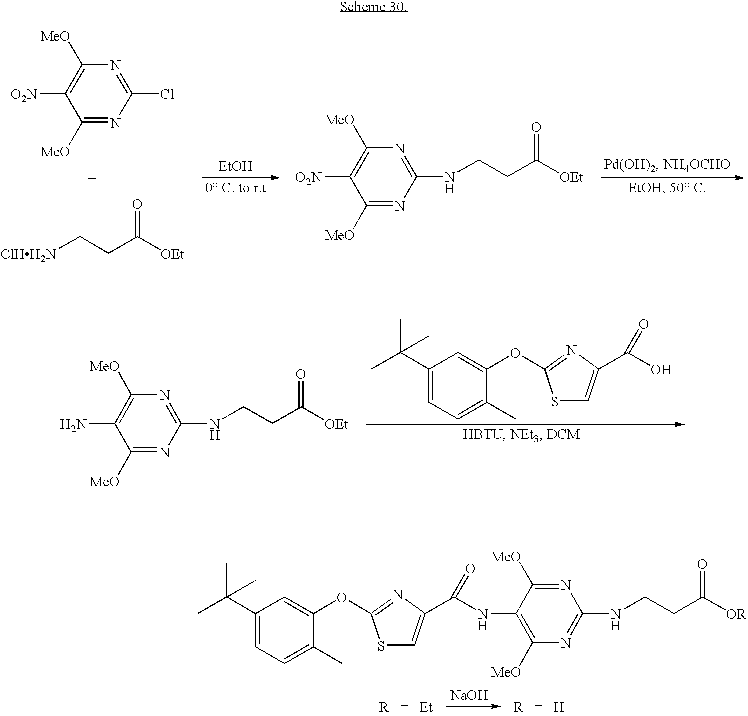

It will be understood that the processes detailed above are solely for the purpose of illustrating the invention and should not be construed as limiting. A process utilising similar or analogous reagents and/or conditions known to one skilled in the art may also be used to obtain a compound of the invention.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

The activity and selectivity of the compounds may be determined by any suitable assay known in the art.

The compounds of the invention may be used in the treatment of numerous ailments, conditions and diseases including, but not limited thereto, cancer, endometriosis, uterine myoma, an ovarian disease, a mammary cystic disease, prostatic hypertrophy, amenorrhea, precocious puberty, premenstrual syndrome, a sex-steroid-dependent pathophysiology, benign prostatic hyperplasia, Alzheimer's disease, HIV infection, AIDS and diseases caused by thyroid malfunction, or to arrest spermatogenesis.

The term “cancer” as used herein refers to any disease or condition characterised by uncontrolled, abnormal growth of cells and includes all known types of cancer, for example cancer of the bladder, breast, colon, brain, bone, head, blood, eye, neck, skin, lungs, ovaries, prostate and rectum; digestive, gastrointestinal, endometrial, hematological, AIDS-related, muscoskeletal, neurological and gynecological cancers; lympomas, melanomas and leukaemia.

In therapeutic use, the active compound may be administered orally, rectally, intra-vaginally, parenterally, by inhalation (pulmonary delivery), topically, ocularly, nasally, or to the buccal cavity. Oral administration is preferred. Thus, the therapeutic compositions of the present invention may take the form of any of the known pharmaceutical compositions for such methods of administration. The compositions may be formulated in a manner known to those skilled in the art so as to give a controlled release, for example rapid release or sustained release, of the compounds of the present invention. Pharmaceutically acceptable carriers suitable for use in such compositions are well known in the art. The compositions of the invention may contain 0.1-99% by weight of active compound. The compositions of the invention are generally prepared in unit dosage form. Preferably, a unit dose comprises the active ingredient in an amount of 1-500 mg. The excipients used in the preparation of these compositions are the excipients known in the art.

Appropriate dosage levels may be determined by any suitable method known to one skilled in the art. It will be understood, however, that the specific dose level for any particular patient will depend upon a variety of factors including the activity of the specific compound employed, the age, body weight, general health, sex, diet, time of administration, route of administration, rate of excretion, drug combination and the severity of the disease undergoing treatment.

Compositions for oral administration are preferred compositions of the invention and there are known pharmaceutical forms for such administration, for example tablets, capsules, granules, syrups and aqueous or oily suspensions. The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable sweetening, flavouring and colouring agents may also be present.

The pharmaceutical compositions of the invention may also be in the form of oil-in-water emulsions. The oily phase may be a vegetable oil, for example olive oil or arachis oil, or a mineral oil, for example liquid paraffin, or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid, find use in the preparation of injectables.

The compounds of the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for topical administration are also suitable for use in the invention. The pharmaceutically active compound may be dispersed in a pharmaceutically acceptable cream, ointment or gel. A suitable cream may be prepared by incorporating the active compound in a topical vehicle such as light liquid paraffin, dispersed in a aqueous medium using surfactants. An ointment may be prepared by mixing the active compound with a topical vehicle such as a mineral oil or wax. A gel may be prepared by mixing the active compound with a topical vehicle comprising a gelling agent. Topically administrable compositions may also comprise a matrix in which the pharmaceutically active compounds of the present invention are dispersed so that the compounds are held in contact with the skin in order to administer the compounds transdermally.

The following Examples illustrate the invention.

In the Examples and Intermediates, all syntheses were carried out under dry nitrogen. Tetrahydrofuran (THF), diethyl ether, dichloromethane (DCM), toluene, N,N-dimethylaminoformamide (DMF), and m-xylene were dried and purified according to standard procedures and stored under nitrogen (as described in The Purification of Laboratory Chemicals, D. D. Perrin, W. L. F. Armarego, D. R. Perrin and C. Chai, Butterworth-Heinemann, 2003). Light petroleum refers to the fraction with b. p. 40-60° C. DMSO refers to dimethylsulfoxide. HBTU refers to O-benzotriazol-1-yl-N,N,N′,N′-tetramethyluronium hexafluorophosphate, EDC refers to 1-(3-dimethylaminopropyl)-3-ethyl-carbodiimide hydrochloride and HATU refers to 2-(7-Aza-1H-benzotriazole-1-yl)-1,1,3,3-tetramethyluronium hexafluorophosphate. Thin layer chromatography (TLC) was performed on silica (SiO₂) plates. ¹H NMR spectra were generated at 400 MHz in CDCl₃ unless otherwise stated. Mass spectral data were generated on a Waters ZQ instrument.

Preparative HPLC was carried out using an XBridgePrep C₁₈ 5 μm OBD column with dimensions 19×100 mm on an Agilent 1100 series instrument.

Method: LCMSXTERRAMETHOD
Acetonitrile (B)/Water + NH₄OH (A) (2.5 mL in 2.5 L water)
Time: 15 min

| Time (min) | % B Acetonitrile | Flow (mL/min) |
|---|---|---|
| 0.00 | 5.0 | 20.00 |
| 10.00 | 95.0 | 20.00 |
| 12.00 | 5.0 | 20.00 |
| 15.00 | 5.0 | 20.00 |

### Intermediate 1: Ethyl 2-(3-tert-butylphenoxy)oxazole-4-carboxylate

To a solution of 3-tert-butyl phenol (1.00 g, 6.65 mmol) in dry THF (25 ml) was added potassium tert-butoxide (747 mg, 6.65 mmol) and the mixture was heated to reflux for 1 h. Upon cooling, the volatiles were removed in vacuo before the residue was taken up into DMSO (20 ml). Ethyl 2-chlorooxazole-4-carboxylate (prepared according to the procedure of K. J. Hodgetts and M. T. Kershaw, Organic Letters, 2002, 4, 2905-2907; 993 mg, 5.65 mmol) was then added before the mixture was heated to 85° C. for 16 h. Upon cooling the reaction mixture was partitioned between ethyl acetate (25 ml) and water (25 ml) and after rigorous shaking the aqueous layer was separated and extracted further with ethyl acetate (3×25 ml). The combined organic phase was washed with brine (50 ml) and dried (MgSO₄) before being concentrated in vacuo. The crude product was then purified by column chromatography (SiO₂; 19:1 light petroleum-ethyl acetate to 9:1) to afford the title compound as a pale yellow oil (1.43 g, 87%). ¹H NMR δ 7.84 (1H, s), 7.20-7.30 (3H, m), 7.10-7.15 (1H, m), 4.27 (2H, q), 1.27 (3H, t) and 1.26 (9H, s).

### Intermediate 2: Ethyl 2-(3-tert-butylphenoxy)thiazolecarboxylate

To a solution of 3-tert-butyl phenol (100 mg, 0.65 mmol) in dry THF (5 ml) was added potassium tert-butoxide (75 mg, 0.65 mmol) and the mixture was heated to reflux for 1 h. Upon cooling, the volatiles were removed in vacuo before the residue was taken up into dimethylsulfoxide (DMSO; 20 ml). Ethyl 2-chlorothiazole-4-carboxylate (prepared according to the procedure of T. R. Kelly and F. Lang, J. Org. Chem., 1996, 61, 4623-4633; however in our hands the material produced by this synthesis was a 2:1 mixture of the 2-chloro- and 5-chloro-thiazole-4-carboxylates; 130 mg, 0.67 mmol) was then added before the mixture was heated to 85° C. for 16 h. Upon cooling the reaction mixture was partitioned between ethyl acetate (25 ml) and water (25 ml) and after rigorous shaking the aqueous later was separated and extracted further with ethyl acetate (3×25 ml). The combined organic phase was washed with brine (50 ml) and dried (MgSO₄) before being concentrated in vacuo. The crude product was then purified by column chromatography (SiO₂; 49:1 light petroleum-ethyl acetate to 19:1) to afford the title compound as a pale yellow oil (100 mg, 50%). ¹H NMR δ 8.20 (1H, s), 7.20-7.27 (3H, m), 7.01-7.05 (1H, m), 4.31 (2H, q), 1.31 (3H, t) and 1.26 (9H, s).

### Intermediate 3: N-(2-(dimethylamino)ethyl)-4,6-dimethoxy-5-nitropyrimidin-2-amine

To a cooled (0° C.) solution of N,N-dimethylethylenediamine (12.6 ml, 115 mmol) in ethanol (200 ml) was added portion-wise 2-chloro-4,6-dimethoxy-5-nitropyrimidine (Intermediate 13; 5 g, 23 mmol) over a period of ca. 10 min. The mixture was stirred for a further hour at 0° C. [reaction complete according to analysis (TLC)]. The ethanol was then removed under reduced pressure and the resulting yellow oil was dissolved in dichloromethane (500 ml) and the solution was washed with saturated aqueous sodium bicarbonate solution (200 ml). The organic solution was separated, dried (MgSO₄) and concentrated under reduced pressure to give a yellow oil. This material was purified by recrystallisation from 3:1 petrol and ethyl acetate to afford a yellow crystalline solid (3.8 g, 61%). The mother liquors were purified by column chromatography (SiO₂, 9:1-methanol in dichloromethane) to give a further quantity of the title product (0.72 g; total 4.52 g, 73%). R_{f} 0.33 (9:1-dichloromethane-methanol). ¹H NMR δ 4.91 (1H, br, s), 4.04 (3H, s), 3.95 (3H, s), 3.45-3.54 (2H, m), 2.51-2.56 (2H, m) and 2.29 (6H, s).

### Intermediate 4: N-(2-(dimethylamino)ethyl)4,6-dimethoxypyrimidine-2,5-diamine

To a solution of N-(2-(dimethylamino)ethyl)-4,6-dimethoxy-5-nitropyrimidin-2-amine (Intermediate 3; 240 mg, 0.89 mmol) was added zinc powder (575 mg, 8.86 mmol, 10 equiv.) and the resulting mixtures was sonicated at room temperature for 3 h. Analysis of the reaction mixture by TLC indicated incomplete reaction (SiO₂ plate, 2:8 methanol-dichloromethane-plate eluted twice) so sonication was continued for 5 h. The solid material was then removed by vacuum filtration and after washing with ethyl acetate the volatiles were removed in vacuo. The residue was taken up into dichloromethane (50 ml), washed with saturated aqueous sodium bicarbonate solution (100 ml), dried (MgSO₄) and concentrated (136 mg, 64%). R_{f} 0.32 (TLC conditions as above). ¹H NMR δ 4.91 (1H, br, s), 3.92 (6H, s), 3.41-3.47 (2H, m), 2.85 (2H, br, s), 2.46-2.51 (2H, m) and 2.29 (6H, s).

### Intermediate 5. 3,3-dimethyl-2,3-dihydro-1H-inden-5-ol

To a solution of phenol (60 g, 638 mmol) in meta-xylene (200 ml) was added 85% phosphoric acid (18 ml, 134 mmol) and phosphorous pentoxide (18 g, 127 mmol) before the mixture was stirred vigorously and heated to 110° C. To this was added isoprene (60 ml, 600 mmol) drop-wise over 30 min. Heating was continued for a further 60 min before the reaction was cooled to ambient temperature. The mixture was partitioned between water (250 ml) and ethyl acetate (250 ml) and the aqueous layer was separated and then washed with further portions of ethyl acetate (2×250 ml). The combined organic material was washed with brine (250 ml) and then dried (MgSO₄) before being concentrated in vacuo. The material was purified by dry flash chromatography (SiO₂, pentane-ethyl acetate) and the material concentrated to give an orange liquid. The product crystallised overnight and the crystals were collected by filtration and washed with cyclohexane to afford the title compound as pale pink needle-like crystals (1.7 g, 2%). ¹H NMR (DMSO) 9.04 (1H, s), 6.94 (1H, d), 6.53-6.50 (2H, m), 2.71 (2H, t), 1.83 (2H, t), 1.17 (6H, s).

### Intermediate 6. Ethyl 2-(3,3-dimethyl-2,3-dihydro-1H-inden-5-yloxy)oxazole-4-carboxylate

To a solution of 3,3-dimethyl-2,3-dihydro-1H-inden-5-ol (Intermediate 5; 430 mg, 2.63 mmol) in dry THF (10 ml) was added potassium tert-butoxide (295 mg, 2.63 mmol) and the mixture was heated to 70° C. for 1 h. Upon cooling, the volatiles were removed in vacuo before the residue was taken up into DMSO (10 ml). Ethyl 2-chlorooxazole-4-carboxylate (prepared according to the procedure of K. J. Hodgetts and M. T. Kershaw, Organic Letters, 2002, 4, 2905-2907; 400 mg, 2.27 mmol) was then added before the mixture was heated to 85° C. for 16 h. Upon cooling the reaction mixture was partitioned between ethyl acetate (25 ml) and water (25 ml) and after rigorous shaking the aqueous layer was separated and extracted further with ethyl acetate (3×25 ml). The combined organic phase was washed with brine (50 ml) and dried (MgSO₄) before being concentrated in vacuo. The crude product was then purified by column chromatography (SiO₂; 19:1 pentane-ethyl acetate) to afford the title compound as a yellow oil (quantitative yield). ¹H NMR (DMSO) 8.55 (1H, s), 7.27-7.29 (1H, m), 7.14-7.2 (2H, m), 4.24 (2H, 4.24), 2.87 (2H, t), 1.93 (2H, t), 1.25 (3H, t), 1.23 (6H, s).

### Intermediate 7. Ethyl 2-(3,3-dimethyl-2,3-dihydro-1H-inden-5-yloxy)thiazole-4-carboxylate

Prepared from 3,3-dimethyl-2,3-dihydro-1H-inden-5-ol (Intermediate 5) and ethyl 2-chlorothiazole-4-carboxylate by the same procedure as for ethyl 2-(3,3-dimethyl-2,3-dihydro-1H-inden-5-yloxy)oxazole-4-carboxylate (Intermediate 6). Title compound isolated as a pale brown oil (quantitative yield). ¹H NMR (DMSO) 8.01 (1H, s), 7.29-7.31 (1H, m), 7.21-7.22 (2H, m), 4.26 (2H, q), 2.88 (2H, t), 1.9 (2H, t), 1.28 (3H, t), 1.23 (6H, s).

### Intermediate 8. 5-tert-Butyl-2-methylnitrobenzene

The title compound was prepared from 1-tert-butyl-4-methylbenzene according to the literature procedure of S. A. Shackelford et al., J. Org. Chem., 2003, 68, 267-275. The title compound was isolated as a brown liquid (91%).

### Intermediate 9. 5-tert-butyl-2-methylbenzenamine

5-tert-Butyl-2-methylnitrobenzene (Intermediate 8; 2.6 g, 13.5 mmol) was dissolved in ethanol (40 ml). The solution was flushed with nitrogen and then charged with palladium on charcoal (10% Pd on C, 500 mg) before the flask was evacuated and flushed with hydrogen. The suspension was stirred at ambient temperature for 18 h and then the reaction was flushed with nitrogen and filtered through a pad of filter aid. The volatiles were removed in vacuo to give the title compound as a pale brown oil (2.2 g, 98%). ¹H NMR (DMSO) 6.72-6.74 (1H, m), 6.56-6.57 (1H, m), 6.39-6.42 (1H, m), 4.6 (2H, br, s), 1.92 (3H, s) and 1.13 (9H, s).

### Intermediate 10. 5-tert-butyl-2-methylphenol

A solution of 5-tert-butyl-2-methylbenzenamine (Intermediate 9; 2.2 g, 13 mmol) in sulphuric acid (15%, 10 0 ml) was heated to 70° C. before a solution of sodium nitrite (1 g, 14.6 mmol) in water (20 ml) was added portion-wise. The mixture was heated to 70° C. for a further 90 minutes before being allowed to cool to ambient temperature. The mixture was then partitioned between water (50 ml) and ethyl acetate (50 ml) and the aqueous layer was separated and washed with further portions of ethyl acetate (2×25 ml). The combined organic material was washed with brine (50 ml) and dried (MgSO₄) before being concentrated in vacuo. The title compound was isolated as a brown liquid (2.1 g, quantitative yield). ¹H NMR (DMSO) 9.03 (1H, s), 6.93-6.95 (1H, m), 6.79-6.80 (1H, m), 6.93-6.95 (1H, m), 2.06 (3H, s), and 1.22 (9H, s).

### Intermediate 11. Ethyl 2-(5-tert-butyl-2-methylphenoxy)oxazole-4-carboxylate

Prepared from 5-tert-butyl-2-methylphenol (Intermediate 10) and ethyl 2-chlorooxazole-4-carboxylate by the same procedure as for ethyl 2-(3,3-dimethyl-2,3-dihydro-1H-inden-5-yloxy)oxazole-4-carboxylate (Intermediate 6). The title compound was isolated as a brown oil (39%). ¹H NMR (DMSO) 8.55 (1H, s), 7.35 (1H, m), 7.3 (2H, m), 4.24 (2H, q), 2.13 (3H, s), 1.27 (9H, s), and 1.22 (3H, t).

### Intermediate 12. Ethyl 2-(5-tert-butyl-2-methylphenoxy)thiazole-4-carboxalate

Prepared from 5-tert-butyl-2-methylphenol (Intermediate 10) and ethyl 2-chlorothiazole-4-carboxylate by the same procedure as for ethyl 2-(3,3-dimethyl-2,3-dihydro-1H-inden-5-yloxy)oxazole-4-carboxylate (Intermediate 6). The title compound was isolated as a brown oil (41%). ¹H NMR (DMSO) 7.99 (1H, s), 7.35 (1H, m), 7.32 (2H, m), 4.26 (2H, q), 2.15 (3H, s), 1.27 (9H, s), and 1.27 (3H, t).

### Intermediate 13. 2-chloro-4,6-dimethoxy-5-nitropyrimidine

Prepared according to WO-A-02/098363 or according to the following procedures.

Method A: 2-Chloro-4,6-dimethoxypyrimidine (50 g, 0.29 mol) was suspended in trifluoroacetic anhydride (175 ml, 1.24 mol, 4 equiv.) and the mixture was cooled in a brine-ice bath to +2° C. Concentrated nitric acid was then added drop-wise to the stirred mixture (CARE; effervescence and exotherm) at such a rate that the exotherm did not cause the temperature of the reaction mixture to rise above +40° C. (about 45 minutes). After 1 hour the reaction was complete (as determined by TLC of an aliquot which was quenched with water and extracted with DCM; plate eluted with DCM). The resulting thick white precipitate was poured onto ice (500 g) and the aqueous solution was extracted with dichloromethane (2×400 ml). The combined organic layers were then washed with saturated aqueous sodium bicarbonate solution (2×200 ml) until the washings remained at pH 7. The solution was dried (MgSO₄) and the solvent was removed under reduced pressure to give a yellow-white crystalline solid. This material was partially dissolved in warm diethyl ether (˜300 ml) with stirring and then cooled in an ice-bath. Filtration gave the product as a pale yellow-white crystalline solid (56 g, 88%).

Method B: 2-Chloro-4,6-dimethoxypyrimidine (2 g, 11.5 mol) was suspended in trifluoroacetic anhydride (3.2 ml, 23 mmol, 2 eq.) and the mixture was cooled in a brine ice-bath to +2° C. Fuming nitric-acid (0.58 ml, 13.8 mmol, 1.2 eq) was then added drop-wise to the stirred mixture (exotherm) at such a rate that the exotherm did not cause the temperature of the reaction mixture to rise above +40° C. (about 1 minute). The thick precipitate was then warmed to room temperature and left overnight. After this time the reaction was complete (as determined by TLC of an aliquot which was quenched with water and extracted with DCM; TLC run with DCM and visualised with uv). Water (20 ml) was added to the resulting thick white precipitate and the aqueous solution was extracted with dichloromethane (2×40 ml). The combined organic layers were then washed with saturated aqueous NaHCO₃ solution (˜20 ml) until the washings remained at pH 7, dried (MgSO₄) and the solvent was removed under reduced pressure to give a white crystalline solid (2.38 g, 95%).

### Intermediate 14. tert-Butyl 2-(4,6-dimethoxy-5-nitropyrimidin-2-ylamino)ethyl(methyl)carbamate

To a cooled (−5° C.) solution of N-(2-aminoethyl)-N-methylcarbamic acid-tert-butyl ester (5 g, 28.7 mmol) in ethanol (60 ml) was added triethylamine. 2-Chloro-4,6-dimethoxy-5-nitropyrimidine (Intermediate 13; 4 g, 18.2 mmol) was added portion-wise over a period of ca. 10 min. The mixture was stirred for a further hour at 0° C. (reaction complete according to analysis (TLC). The ethanol was then removed under reduced pressure and the resulting yellow oil was purified by column chromatography (SiO₂, 1:2 ethyl acetate: petroleum ether) to give a yellow solid (3.76 g; 58%). m/z 358.21 (MH⁺).

### Intermediate 15. tert-Butyl 2-(5-amino-4,6-dimethoxypyrimidin-2-ylamino)ethyl(methyl)carbamate

tert-Butyl 2-(4,6-dimethoxy-5-nitropyrimidin-2ylamino)ethyl(methyl) carbamate (Intermediate 14; 1 g, 2.8 mmol) was dissolved in ethanol (30 ml). The solution was flushed with nitrogen and then charged with palladium on charcoal (10% Pd on C, 150 mg) before the flask was evacuated and flushed with hydrogen. The suspension was stirred at ambient temperature for 18 h and then the reaction was flushed with nitrogen and filtered through a pad of filter aid. The volatiles were removed in vacuo to give the title compound as a pale brown oil (0.9 g, 98%). m/z 328.24 (MH⁺).

### Intermediate 16. 2-(5-tert-Butyl-2-methylphenoxy)oxazole-4-carboxylic acid

To a solution of ethyl 2-(5-tert-butyl-2-methylphenoxy)oxazole-4-carboxylate (Intermediate 11; 1.13 g, 3.74 mmol) dissolved in tetrahydrofuran (15 ml) was added a solution of sodium hydroxide (180 mg, 4.5 mmol) in water (10 ml). The solution was stirred for a further 18 h at ambient temperature whereby the reaction was seen to be complete by TLC analysis. The material was acidified to pH 2 with concentrated hydrochloric acid and then partitioned between ethyl acetate (25 ml) and water (25 ml). After rigorous shaking the aqueous layer was separated and extracted further with ethyl acetate (2×25 ml). The combined organic phase was washed with brine (50 ml) and dried (MgSO₄) before being concentrated in vacuo to give an off white solid (1.03 g; 100%). m/z 276.19 (MH⁺).

### Intermediate 17. tert-Butyl 2-(5-(2-(5-tert-butyl-2-methylphenoxy)oxazole-4-carboxamido)-4,6-dimethoxypyrimidin-2-ylamino)ethyl(methyl)carbamate

To a solution of 2-(5-tert-butyl-2-methylphenoxy)oxazole-4-carboxylic acid (Intermediate 16; 0.385 g, 1.4 mmol), tert-butyl 2-(5-amino-4,6-dimethoxypyrimidin-2-ylamino)ethyl(methyl)carbamate (Intermediate 15; 0.458 g, 1.4 mmol) and triethylamine (0.394 ml, 2.8 mmol) in dichloromethane (20 ml) was added HATU (0.586 g, 1.54 mmol]. The solution was stirred for a further 18 h at ambient temperature whereby the reaction was seen to be complete by TLC analysis. The volatiles were removed in vacuo and the title compound was isolated by chromatography (SiO₂, elution 1:19 MeOH-DCM) as a brown oil (0.45 g, 55%). ¹H NMR δ 7.91 (1H, s), 7.54 (1H, br, s), 7.22-7.31 (3H, m), 3.87 (6H, br, s), 3.45-3.51 (4H, m), 2.93 (3H, br, s), 2.27 (3H, s), 1.46 (9H, br, s) and 1.34 (9H, s).

### Intermediate 18. 2-(5-tert-Butyl-2-methylphenoxy)thiazole-4-carboxylic acid

Prepared from ethyl 2-(5-tert-butyl-2-methylphenoxy)thiazole-4-carboxylate (Intermediate 12; 1.87 g, 5.9 mmol) by the same procedure as for 2-(5-tert-butyl-2-methylphenoxy)oxazole-4-carboxylic acid (Intermediate 16). The title compound was isolated as an off-white solid (1.71 g, 100%). m/z 292.17 (MH⁺).

### Intermediate 19. tert-Butyl 2-(5-(2-(5-tert-butyl-2-methylphenoxy)thiazole-4-carboxamido)-4,6-dimethoxypyrimidin-2-ylamino)ethyl(methyl)carbamate

Prepared from 2-(5-tert-butyl-2-methylphenoxy)thiazole-4-carboxylic acid (Intermediate 18; 0.407 g, 1.4 mmol) and tert-butyl 2-(5-amino-4,6-dimethoxypyrimidin-2-ylamino)ethyl(methyl)carbamate (intermediate 15; 0.458 g, 1.4 mmol) according to the method outlined for Intermediate 17. The title compound was isolated as a brown oil (0.55 g, 65%). ¹H NMR 7.97 (1H, br, s), 7.66 (1H, s), 7.26-7.28 (3H, m), 3.89 (6H, br, s), 3.46-3.52 (4H, m), 2.94 (3H, br, s), 2.28 (3H, s), 1.47 (9H, br, s) and 1.33 (9H, s)

### Intermediate 20. Ethyl 2-(3,3,6-trimethyl-2,3-dihydro-1H-inden-5-yloxy)oxazole-4-carboxylate

The title compound was prepared according to the method outlined for Intermediate 1 from 3,3,6-trimethyl-2,3-dihydro-1H-inden-5-ol (prepared according to WO 03/106446; 0.45 g, 2.56 mmol) and ethyl 2-chlorooxazole-4-carboxylate (prepared according to the procedure of K. J. Hodgetts and M. T. Kershaw, Organic Letters, 2002, 4, 2905-2907; 0.45 g, 2.56 mmol) and isolated as an off-white solid (0.64 g, 80%). m/z 316.20 (MH⁺).

### Intermediate 21. 2-(3,3,6-Trimethyl-2,3-dihydro-1H-inden-5-yloxy)oxazole-4-carboxylic acid

Prepared from ethyl 2-(3,3,6-trimethyl-2,3-dihydro-1H-inden-5-yloxy)oxazole-4-carboxylate (Intermediate 20; 0.63 g, 2.03 mmol) by the same procedure as for 2-(5-tert-butyl-2-methylphenoxy)oxazole-4-carboxylic acid (Intermediate 16). The title compound was isolated as an off-white solid (0.58 g, 99%). m/z 288.14 (MH⁺).

### Intermediate 22. tert-Butyl 2-(4,6-dimethoxy-5-(2-(3,3,6-trimethyl-2,3-dihydro-1H-inden-5-yloxy)oxazole-4-carboxamido)pyrimidin-2-ylamino)ethyl(methyl)carbamate

Prepared from 2-(3,3,6-trimethyl-2,3-dihydro-1H-inden-5-yloxy)oxazole-4-carboxylic acid (Intermediate 21; 0.426 g, 1.48 mmol) and tert-butyl 2-(5-amino-4,6-dimethoxypyrimidin-2-ylamino)ethyl(methyl)carbamate (Intermediate 15; 0.485 g, 1.48 mmol) according to the method outlined for Intermediate 17. The title compound was isolated as an off-white solid (0.54 g, 61%). ¹H NMR 7.91 (1H, s), 7.52 (1H, br, s), 7.12 (1H, br, s), 7.03 (1H, br, s) 3.87 (6H, br, s), 3.53 (2H, m), 3.48 (2H, m), 2.93 (3H, br, s), 2.9 (2H, t), 2.26 (3H, s), 1.98 (2H, t), 1.46 (9H, br, s) and 1.28 (6H, s). m/z 597.37 (MH⁺).

### Intermediate 23. Ethyl 2-(3,3,6-trimethyl-2,3-dihydro-1H-inden-5-yloxy)thiazole-4-carboxylate

The title compound was prepared according to the method outlined for Intermediate 1 from 3,3,6-trimethyl-2,3-dihydro-1H-inden-5-ol (prepared according to WO 03/106446, 0.39 g, 2.24 mmol) and ethyl 2-chlorothiazole-4-carboxylate (prepared according to the procedure of T. R. Kelly and F. Lang, J. Org. Chem., 1996, 61, 4623-4633; however in our hands the material produced by this synthesis was a 2:1 mixture of the 2-chloro- and 5-chloro-thiazole-4-carboxylates; 0.43 g, 2.24 mmol) and isolated as an off-white solid (0.44 g, 60%). m/z 332.15 (MH⁺).

### Intermediate 24. 2-(3,3,6-Trimethyl-2,3-dihydro-1H-inden-5-yloxy)thiazole-4-carboxylic acid

Prepared from ethyl 2-(3,3,6-trimethyl-2,3-dihydro-1H-inden-5-yloxy)thiazole-4-carboxylate (Intermediate 23, 0.44 g, 1.33 mmol) by the same procedure as for 2-(5-tert-butyl-2-methylphenoxy)oxazole-4-carboxylic acid (Intermediate 16). The title compound was isolated as an off-white solid (0.364 g, 91%). m/z 304.12 (MH+).

### Intermediate 25. tert-Butyl 2-(4,6-dimethoxy-5-(2-(3,3,6-trimethyl-2,3-dihydro-1H-inden-5-yloxy)thiazole-4-carboxamido)pyrimidin-2-ylamino)ethyl(methyl)carbamate

Prepared from 2-(3,3,6-trimethyl-2,3-dihydro-1H-inden-5-yloxy)thiazole-4-carboxylic acid (Intermediate 24; 0.36 g, 1.19 mmol) and tert-butyl 2-(5-amino-4,6-dimethoxypyrimidin-2-ylamino)ethyl(methyl)carbamate (Intermediate 15; 0.39 g, 1.19 mmol) according to the method outlined for Intermediate 17. The title compound was isolated as an off-white solid (0.437 g, 59%). m/z 613.31 (MH⁺).

### Intermediate 26. 2-Chlorothiazole-4-carboxylic acid

Prepared from ethyl 2-chlorothiazole-4-carboxylate (1 g, 5.2 mmol) by the same procedure as for 2-(5-tert-butyl-2-methylphenoxy)oxazole-4-carboxylic acid (Intermediate 16). The title compound was isolated as an off-white solid (0.87 g, 100%). m/z 164.01 (MH⁺).

### Intermediate 27. tert-Butyl 2-(5-(2-chlorothiazole-4-carboxamido)-4,6-dimethoxypyrimidin-2-ylamino)ethyl(methyl)carbamate

Prepared from 2-chlorothiazole-4-carboxylic acid (Intermediate 26; 0.25 g, 1.48 mmol) and tert-butyl 2-(5-amino-4,6-dimethoxypyrimidin-2-ylamino)ethyl(methyl)carbamate (Intermediate 15; 0.485 g, 1.48 mmol) according to the method outlined for Intermediate 17. The title compound was isolated as an off-white solid (0.65 g, 92%). ¹H NMR 8.08 (1H, s), 8.01 (1H, br, s), 3.9 (6H, br, s), 3.55 (2H, m), 3.49 (2H, q), 2.94 (3H, br, s), 1.47 (9H, s). m/z 473.10 (MH⁺).

### Intermediate 28. tert-Butyl 2-(5-(2-(5-isopropyl-2-methyl-henoxy)thiazole-4-carboxamido)-4,6-dimethoxypyrimidin-2-ylamino)ethyl(methyl)carbamate

The title compound was prepared according to the method outlined for Intermediate 1 from 5-isopropyl-2-methylphenol (0.197 g, 1.31 mmol) and tert-butyl 2-(5-(2-chlorothiazole-4-carboxamido)-4,6-dimethoxypyrimidin-2-ylamino)ethyl(methyl)carbamate (Intermediate 27; 0.56 g, 1.2 mmol) and isolated as a brown oil (0.7 g, 100%). m/z 587.32 (MH⁺).

### Intermediate 29. 2-(4,6-Dimethoxy-5-nitropyrimidin-2-ylamino)ethanol

2-Amino ethanol (33 g, 0.55 mol, 3 eq.) was dissolved in ethanol (600 ml) and the solution was cooled in an ice-bath. The chloropyrimidine (40 g, 0.18 mol) was added portion-wise over 15 minutes and the solution was warmed to room temperature and stirred overnight. After this time the reaction was complete by tlc (3:1 petrol-EtOAc, uv, KMnO₄, product is a yellow spot). The ethanol was removed under reduced pressure and the residue was partitioned between ethyl acetate (500 ml) and sat. sodium bicarbonate solution (400 ml). The layers were separated and the aqueous layer was extracted with ethyl acetate (1×300 ml), the combined organic layers were then dried (MgSO₄) and the solvent was removed under reduced pressure to give a bright yellow solid. This was triturated with ether/petrol to give the product as a bright yellow powder (35 g, 80%). ¹H NMR (DMSO); 3.4 (2H, q, CH₂N), 3.56 (2H, q, CH₂O), 3.90 (3H, s, OCH₃), 3.94 (3H, s, OCH₃), 4.71 (1H, t, OH), 8.11 (1H, t, NH).

### Intermediate 30. N-(2-(tert-Butyldimethylsilyloxy)ethyl)-4,6-dimethoxy-5-nitropyrimidin-2-amine

2-(4,6-Dimethoxy-5-nitropyrimidin-2-ylamino)ethanol (Intermediate 29; 35 g, 143 mmol) was partially dissolved in dichloromethane (500 ml) and the mixture was cooled in an ice-bath. Imidazole (14.4 g, 214 mmol, 1.5 eq.) was added, followed by tert-butyldimethylsilyl chloride (TBDMS-Cl; 23.7 g, 157 mmol, 1.1 eq.) and the solution was warmed to room temperature and stirred vigorously overnight, after which time a thick white precipitate of imidazole hydrochloride had formed and the reaction was complete (TLC; 3:1 petrol-ether, uv, KMnO₄, product is a yellow spot). Water (500 ml) was added and the layers were separated, the organic layer was washed with water (300 ml), dried (MgSO₄) and the solvent was removed under reduced pressure to give yellow solid. This material was triturated with petrol/ether to give the product as a fine yellow powder (35 g, 75%). ¹H NMR (DMSO); 0.0 (s, 6H, 2×CH₃Si), 0.81 (9H, s, (CH₃)₃CSi), 3.4 (2H, q, CH₂N), 3.68 (2H, t, CH₂O); 3.87, (3H, s, OCH₃), 3.90 (3H, s, OCH₃), 8.16 (1H, t, NH).

### Intermediate 31. N2-(2-(tert-Butyldimethylsilyloxy)ethyl)-4,6-dimethoxypyrimidine-2,5-diamine

N-(2-(tert-butyldimethylsilyloxy)ethyl)-4,6-dimethoxy-5-nitropyrimidin-2-amine (Intermediate 30; 0.65 g, 1.8 mmol) was dissolved in ethanol (30 ml). The solution was flushed with nitrogen and then charged with palladium on charcoal (10% Pd on C, 200 mg) before the flask was evacuated and flushed with hydrogen. The suspension was stirred at ambient temperature for 2 h and then the reaction was flushed with nitrogen and filtered through a pad of filter aid. The volatiles were removed in vacuo to give the title compound as a pale brown oil (0.498 g, 84%). ¹H NMR 4.73 (1H, br, t), 3.85 (6H, s), 3.7 (2H, t), 3.41 (2H, q), 2.89 (2H, br, s), 0.85 (9H, s), and 0.0 (6H, s).

### Intermediate 32. 2-(5-tert-Butyl-2-methylphenoxy)-N-(2-(2-(tert-butyldimethylsilyloxy)ethylamino)-4,6-dimethoxypyrimidin-5-yl)thiazole-4-carboxamide

Prepared from 2-(5-tert-butyl-2-methylphenoxy)thiazole-4-carboxylic acid (Intermediate 18; 0.44 g, 1.52 mmol) and N-(2-(tert-butyldimethylsilyloxy)ethyl)-4,6-dimethoxypyrimidine-2,5-diamine (Intermediate 31; 0.498 g, 1.52 mmol) according to the method outlined for Intermediate 17. The title compound was isolated as an off-white solid (0.77 g, 84%). ¹H NMR 7.86 (1H, br, s), 7.55 (1H, s) 7.14-7.19 (3H, m), 5.12 (1H t), 3.8 (6H, s), 3.69 (2H, t), 3.44 (2H, q), 2.18 (3H, s), 1.24 (9H, s), 0.84 (9H, s) and 0.0 (6H, s). m/z 602.30 (MH⁺).

### Intermediate 33. 2-(5-(2-(5-tert-Butyl-2-methylphenoxy)thiazole-4-carboxamido)-4,6-dimethoxypyrimidin-2-ylamino)ethyl methanesulfonate

Methanesulfonyl chloride (0.096 ml, 1.23 mmol) was added to a cooled (0° C.) solution of 2-(5-tert-butyl-2-methylphenoxy)-N-(2-(2-hydroxyethylamino)-4,6-dimethoxypyrimidin-5-yl)thiazole-4-carboxamide (Example 13; 0.3 g, 0.62 mmol) and triethylamine (0.173 ml, 1.23 mmol) in DCM (5 ml). Stirring was continued at 0° C. for a further 10 minutes after which time TLC showed that the reaction had gone to completion. The reaction mixture was partitioned between dichloromethane (25 ml) and water (25 ml) and after rigorous shaking the aqueous later was separated and extracted further with dichloromethane (1×25 ml). The combined organic phase was washed with saturated sodium hydrogen carbonate (50 ml) and dried (MgSO₄) before being concentrated in vacuo. The title compound was isolated as a brown oil (0.35 g, 100%). m/z 566.20 (MH⁺).

### Intermediate 34: 2-(2-tert-Butoxyethoxy)-4,6-dimethoxy-5-nitropyrimidine

Sodium hydride (91 mg, 2.3 mmol) was added to a cooled (0° C.) solution of 2-chloro-4,6-dimethoxy-5-nitropyrimidine (Intermediate 13; 0.5 g, 2.3 mmol) and ethylene glycol mono-tert butyl ether in anhydrous THF (10 ml) and the solution was stirred for 15 minutes. The mixture was divided between water (20 ml) and ethyl acetate (20 ml). The layers were separated, the organic layer was dried (MgSO₄) and the solvent was removed. The crude product was then purified by column chromatography (SiO₂; 4:1 light petroleum-ethyl acetate) to afford the title compound as an off-white solid (335 mg, 50%). ¹H NMR δ 4.49 (2H, t), 4.06 (6H, s), 3.73 (2H, t) and 1.22 (9H, s).

### Intermediate 35: 2-(2-tert-Butoxyethoxy)-4,6-dimethoxypyrimidin-5-amine

2-(2-tert-Butoxyethoxy)-4,6-dimethoxy-5-nitropyrimidine (Intermediate 34; 0.5 g, 1.66 mmol) was dissolved in ethanol (20 ml). The solution was flushed with nitrogen and then charged with palladium on charcoal (10% Pd on C, 100 mg) before the flask was evacuated and flushed with hydrogen. The suspension was stirred at ambient temperature for 5 h and then the reaction was flushed with nitrogen and filtered through a pad of filter aid. The volatiles were removed in vacuo to give the title compound as a pale brown oil (430 mg, 100%). ¹H NMR δ 4.36 (2H, t), 3.97 (6H, s), 3.72 (2H, t), 3.14 (2H, br s) and 1.22 (9H, s).

### Intermediate 36: N-(2-(2-(tert-Butyldimethylsilyloxy)ethylamino)-4,6-dimethoxypyrimidin-5-yl)-2-chlorothiazole-4-carboxamide

EDC (3.37 g, 18.11 mmol) was added to a solution of 2-chlorothiazole-4-carboxylic acid (Intermediate 26; 2.82 g, 17.25 mmol), N2-(2-(tert-Butyldimethyl silyloxy)ethyl)-4,6-dimethoxypyrimidine-2,5-diamine (Intermediate 31; 5.7 g, 17.42 mmol), hydroxybenzotriazole (2.45 g, 18.11 mmol) and triethylamine (4.85 g, 34.5 mmol) in DCM (100 ml). The solution was stirred for a further 18 h at ambient temperature and then the mixture was washed with aqueous citric acid solution (5%; 50 ml) followed by washing with saturated aqueous sodium hydrogen carbonate solution (50 ml). The material was washed with brine, dried (MgSO₄) and the solvent was removed in vacuo. The title compound was isolated by column chromatography (SiO₂, elution 1:7 ethyl acetate-petroleum ether) as a brown solid (4.5 g, 55%). ¹H NMR δ 7.99 (1H, s), 7.91 (1H, s), 5.15 (1H, br t), 3.80 (6H, s), 3.69 (2H, t), 3.45 (2H, q), 0.84 (9H, s) and 0.0 (6H, s); m/z 474.12 (MH⁺)

### Intermediate 37: N-(2-(2-(tert-Butyldimethylsilyloxy)ethylamino)-4,6-dimethoxypyrimidin-5-yl)-2-(5-chloro-2-methylphenoxy)thiazole-4-carboxamide

The title compound was prepared according to the method outlined for Intermediate 1 from 5-chloro-2-methylphenol (0.16 g, 1.15 mmol) and N-(2-(2-(tert-butyldimethylsilyloxy)ethylamino)-4,6-dimethoxypyrimidin-5-yl)-2-chlorothiazole-4-carboxamide (Intermediate 36; 0.5 g, 1.05 mmol) with the exception that the phenoxide and the chlorothiazole were heated to 10° C. for 2 hours instead of 85° C. for 16 hours. The compound was isolated as a brown oil (0.57 g, 78%); m/z 580.22 (MH+).

### Intermediate 38: N-(2-(2-(tert-Butyldimethylsilyloxy)ethylamino)-4,6-dimethoxypyrimidin-5-yl)-2-(5-Isopropyl-2-methylphenoxy)thiazole-4-carboxamide

The title compound was prepared according to the method outlined for Intermediate 1 from 5-isopropyl-2-methylphenol (0.17 g, 1.16 mmol) and N-(2-(2-(tert-butyldimethylsilyloxy)ethylamino)-4,6-dimethoxypyrimidin-5-yl)-2-chlorothiazole-4-carboxamide (Intermediate 36; 0.5 g, 1.05 mmol). It was isolated as a pale yellow gum (0.38 g, 62%). ¹H NMR δ 7.93 (1H, s), 7.63 (1H, s), 7.21-7.23 (1H, m), 7.0-7.1 (2H, m), 5.2 (1H, br t), 3.87 (6H, s), 3.75-3.78 (2H, m), 3.50-3.54 (2H, m), 2.90-2.94 (1H, m), 2.26 (3H, s), 1.25 (3H, s), 1.23 (3H, s), 0.91 (9H, s) and 0.07 (6H, s); m/z 588.34 (MH+).

### Intermediate 39: N-(2-(2-(tert-Butyldimethylsilyloxy)ethylamino)-4,6-dimethoxypyrimidin-5-yl)-2-(2,5-dimethylphenoxy)thiazole-4-carboxamide

The title compound was prepared according to the method outlined for Intermediate 1 from 2,5-dimethylphenol (0.142 g, 1.16 mmol) and N-(2-(2-(tert-butyldimethylsilyloxy)ethylamino)-4,6-dimethoxypyrimidin-5-yl)-2-chlorothiazole-4-carboxamide (Intermediate 36; 0.5 g, 1.05 mmol). It was isolated as a yellow gum (0.54 g, 91%). ¹H NMR δ 7.91 (1H, s), 7.64 (1H, s), 7.17-7.19 (1H, m), 7.0-5.1 (2H, m), 5.2 (1H, br t), 3.87 (6H, s), 3.76 (2H, t), 3.50-3.54 (2H, m), 2.35 (3H, s), 2.25 (3H, s), 0.91 (9H, s), and 0.07 (6H, s); m/z 560.31 (MH+).

### Intermediate 40: N-(2-(2-(tert-Butyldimethylsilyloxy)ethylamino)-4,6-dimethoxypyrimidin-5-yl)-2-(o-tolyloxy)thiazole-4-carboxamide

The title compound was prepared according to the method outlined for Intermediate 1 from o-cresol (0.125 g, 1.16 mmol) and N-(2-(2-(tert-butyldimethylsilyloxy)ethylamino)-4,6-dimethoxypyrimidin-5-yl)-2-chlorothiazole-4-carboxamide (Intermediate 36; 0.5 g, 1.05 mmol). It was isolated as a yellow gum (0.41 g, 72%). ¹H NMR δ 7.89 (1H, s), 7.65 (1H, s), 7.23-7.32 (4H, m), 5.2 (1H, br t), 3.87 (6H, s), 3.76 (2H, t), 3.50-3.54 (2H, m), 2.3 (3H, s), 0.91 (9H, s), and 0.07 (6H, s). m/z 546.23 (MH+).

### Intermediate 41: 2-Bromothiazole-4-carboxylic acid

The title compound was prepared from methyl 2-bromothiazole-4-carboxylate (5 g, 22.5 mmol) by the same procedure as for 2-(5-tert-butyl-2-methylphenoxy)oxazole-4-carboxylic acid (Intermediate 16). The compound was isolated as a pale brown gum (4.4 g, 94%); m/z 209.99 (MH⁺).

### Intermediate 42: N-(4,6-Dimethoxy-5-nitropyrimidin-2-yl)-N′-isopropylethane-1,2-diamine

N-Isopropylethylene diamine (0.24 ml, 2 mmol) was dissolved in ethanol (5 ml) at 0° C. and the solution was cooled in an ice-bath. 2-Chloro-4,6-dimethoxy-5-nitropyrimidine (Intermediate 13; 0.40 g, 2 mmol) was added portion-wise over 10 minutes and the solution was allowed to warm to room temperature and then stirring continued for a further 16 hours. The ethanol was removed under reduced pressure and the residue was purified by column chromatography (silica, 0-30% methanol in dichloromethane) to give the product as a yellow solid (0.4 mg, 74%), R_{f}=0.3 (methanol-dichloromethane 1:9); m.p.=135° C. ¹H NMR δ_{H} (DMSO) 1.16 (6H, d), 3.0 (2H, m), 3.17 (1 H, m), 3.58 (2H, m), 3.92 (3H, s), 3.98 (3H, s) and 8.24 (2H, m); MS (ES⁺) m/z 286 (MH⁺).

### Intermediate 43: tert-Butyl-2-(4,6-dimethoxy-5-nitropyrimidin-2-yl amino)ethyl (isopropyl)carbamate

N-(4,6-Dimethoxy-5-nitropyrimidin-2-yl)-N′-isopropylethane-1,2-diamine (Intermediate 42; 1 g, 3.5 mmol) was dissolved in a mixture of dioxan and water (15 ml, 3:1). Di-tert-butyl Bicarbonate (912 mg, 4.2 mmol) was added and a precipitate was seen to form over 1 hour. Stirring was continued for a further 2 hours and the dioxan was then removed in vacuo. The material was dissolved in ethyl acetate (100 ml) and washed with saturated aqueous sodium bicarbonate (2×50 ml) before being dried (MgSO₄) and the solvent removed in vacuo. The crude product was purified by chromatography (silica; petrol-ethyl acetate) to afford the title compound as a pale yellow solid (1.3 g, 98%), m.p. 110° C. ¹H NMR δ_{H} 3.82 (3H, s), 3.74 (3H, s), 3.36 (2H, dd), 3.2 (3H, m), 1.3 (9H, s), 0.96 (3H, s) and 0.94 (3H, s); m/z 386.2 (MH⁺).

### Intermediate 44: tert-Butyl 2-(5-amino-4,6-dimethoxypyrimidin-2-yl amino)ethyl(isopropyl)carbamate

tert-Butyl-2-(4,6-dimethoxy-5-nitro pyrimidin-2-yl amino)ethyl (isopropyl) carbamate (Intermediate 13; 9 g, 23.4 mmol) was dissolved in ethanol (200 ml). The solution was flushed with nitrogen and then charged with palladium on charcoal (10% Pd on C, 1 g) before the flask was evacuated and flushed with hydrogen. The suspension was stirred at room temperature for 18 hours before the reaction was then flushed with nitrogen and filtered through a pad of celite. The volatiles were removed in vacuo to give the title compound as a pale brown oil (7.5 g, 92%); m/z 356.23 (MH⁺).

### Intermediate 45: tert-Butyl 2-(5-(2-bromothiazole-4-carboxamido)-4,6-dimethoxypyrimidin-2-ylamino)ethyl(isopropyl)carbamate

The title compound was prepared from 2-bromothiazole-4-carboxylic acid (Intermediate 41, 4.39 g, 21.1 mmol) and tert-butyl 2-(5-amino-4,6-dimethoxypyrimidin-2-yl amino)ethyl(isopropyl)carbamate (Intermediate 44, 7.5 g, 21 mmol) according to the method outlined for N-(2-(2-(tert-butyldimethylsilyloxy)ethylamino)-4,6-dimethoxypyrimidin-5-yl)-2-chlorothiazole-4-carboxamide (Intermediate 36). It was isolated as a pale brown solid (6.65 g, 58%); m/z 547.13 (MH⁺).

### Intermediate 46: 6-Bromo-3,3-dimethyl-2,3-dihydro-1H-inden-5-ol

The title compound was prepared from 3,3-dimethyl-2,3-dihydro-1H-inden-5-ol (Intermediate 5, 1.62 g, 10 mmol) according to the procedure outlined in patent FR2339591. It was isolated as a pale brown liquid (2.07 g, 86%). ¹H NMR (DMSO) δ 9.83 (1H, s), 7.24 (1H, s), 6.7 (1H, s), 2.72 (2H, t), 1.83 (2H, t), and 1.15 (6H, s); m/z 241.17 (MH⁻).

### Intermediate 47: tert-Butyl 2-(5-(2-(6-bromo-3,3-dimethyl-2,3-dihydro-1H-inden-5-yloxy)thiazole-4-carboxamido)-4,6-dimethoxypyrimidin-2-ylamino)ethyl(isopropyl)carbamate

The title compound was prepared according to the method outlined for Intermediate 1 from 6-bromo-3,3-dimethyl-2,3-dihydro-1H-inden-5-ol (Intermediate 46, 0.27 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol). It was isolated as a yellow gum (0.39 g, 60%); m/z 707.27 (MH+).

### Intermediate 48: 6-Chloro-3,3-dimethyl-2,3-dihydro-1H-inden-5-ol

The title compound was prepared from 3,3-dimethyl-2,3-dihydro-1H-inden-5-ol (Intermediate 5, 4 g, 24.7 mmol) according to the procedure outlined in patent FR2339591. It was isolated as a pale yellow liquid (2.67 g, 55%). ¹H NMR δ 7.11 (1H, s), 6.79 (1H, s), 2.78-2.82 (2H, m), 1.89-1.93 (2H, m), and 1.22 (6H, s); m/z 195.3 (MH⁻).

### Intermediate 49: tert-Butyl 2-(5-(2-(6-chloro-3,3-dimethyl-2,3-dihydro-1H-inden-5-yloxy)thiazole-4-carboxamido)-4,6-dimethoxypyrimidin-2-ylamino)ethyl(isopropyl)carbamate

The title compound was prepared according to the method outlined for Intermediate 1 from 6-chloro-3,3-dimethyl-2,3-dihydro-1H-inden-5-ol (Intermediate 48, 0.22 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol). It was isolated as a yellow gum (0.35 g, 58%); m/z 661.30 (MH+).

### Intermediate 50: 5-Hydroxy-6-methoxy-3,3-dimethyl-2,3-dihydroinden-1-one

The title compound was prepared from 2-methoxyphenol (30 g, 242 mmol) and 3,3-dimethylacrylic acid (19.4 g, 194 mmol) according to the procedure outlined in patent WO 02/098363. It was isolated as a pale yellow liquid (10 g, 25%). ¹H NMR δ 7.13 (1H, s), 6.96 (1H, s), 6.26 (1H, s), 3.93 (3H, s) 2.55 (2H, s) and 1.38 (6H, s); m/z 207.2 (MH+).

### Intermediate 51: 6-Methoxy-3,3-dimethyl-2,3-dihydro-1H-inden-5-ol

The title compound was prepared from 5-hydroxy-6-methoxy-3,3-dimethyl-2,3-dihydroinden-1-one (Intermediate 50, 7.87 g, 38.2 mmol) according to the procedure outlined in patent WO 02/098363. It was isolated as a pale yellow liquid (7.33 g, 100%). ¹H NMR δ 6.72 (1H, s), 5.51 (1H, s), 3.86 (3H, s), 2.8 (2H, t), 1.9 (2H, t) and 1.2 (6H, s); m/z 191.28 (MH⁺).

### Intermediate 52: tert-Butyl 2-(4,6-dimethoxy-5-(2-(6-methoxy-3,3-dimethyl-2,3-dihydro-1H-inden-5-yloxy)thiazole-4-carboxamido)pyrimidin-2-ylamino)ethyl(isopropyl)carbamate

The title compound was prepared according to the method outlined for Intermediate 1 from 6-methoxy-3,3-dimethyl-2,3-dihydro-1H-inden-5-ol (Intermediate 51, 0.21 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol). It was isolated as a yellow gum (0.54 g, 90%); m/z 657.40 (MH+).

### Intermediate 53: 5-Methoxy-2-methylphenol

A solution of 5-methoxy-2-methylbenzenamine (2 g, 14.6 mmol) in sulphuric acid (15%, 50 ml) was heated to 70° C. before a solution of sodium nitrite (1.1 g, 16 mmol) in water (10 ml) was added portion-wise. The mixture was heated to 70° C. for a further 90 minutes before being allowed to cool to ambient temperature. The mixture was then partitioned between water (50 ml) and ethyl acetate (50 ml) and the aqueous layer was separated and washed with further portions of ethyl acetate (2×25 ml). The combined organic material was washed with brine (50 ml) and dried (MgSO₄) before being concentrated in vacuo. The title compound was isolated by column chromatography (petroleum ether-ethyl acetate 3:7) as a purple liquid (0.57 g, 29%). ¹H NMR (DMSO) δ 9.25 (1H, s), 6.91-6.93 (1H, m), 6.34-6.35 (1H, m), 6.25-6.28 (1H, m), 3.64 (3H, s) and 2.02 (3H, s). m/z 137.07 (MH+).

### Intermediate 54: tert-Butyl 2-(4,6-dimethoxy-5-(2-(5-methoxy-2-methylphenoxy)thiazole-4-carboxamido)pyrimidin-2-ylamino)ethyl(isopropyl)carbamate

The title compound was prepared according to the method outlined for Intermediate 1 from 5-methoxy-2-methylphenol (Intermediate 53, 0.15 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol). It was isolated as a pale purple gum (0.3 g, 55%); m/z 603.22 (MH⁺).

### Intermediate 55: 2-Bromo-5-tert-butylphenol

To a cooled solution of 3-tert-butylphenol (5 g, 33.3 mmol) in carbon tetrachloride (CARE! 40 ml) at 5° C. was added bromine (CARE! 1.7 ml, 33.3 mmol) portion-wise over 30 minutes and the reaction was allowed to stir at −5° C. for a further 30 minutes. The reaction was partioned between water and dichloromethane and the organic layer was washed with water until pH neutral before being dried (MgSO₄) and concentrated in vacuo to give the title compound as a yellow liquid (7 g, 92%). ¹H NMR (DMSO) δ 7.35-7.37 (1H, m), 7.05-7.06 (1H, m), 6.83-6.86 (1H, m), 1.29 (9H, s); m/z 227.24/229.22 (MH⁻)

### Intermediate 56: tert-Butyl 2-(5-(2-(2-bromo-5-tert-butylphenoxy)thiazole-4-carboxamido)-4,6-dimethoxypyrimidin-2-ylamino)ethyl(isopropyl)carbamate

The title compound was prepared according to the method outlined for Intermediate 1 from 2-bromo-5-tert-butylphenol (Intermediate 55, 0.23 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol). It was isolated as a pale brown liquid (0.48 g, 75%); m/z 695.25 (MH⁺).

### Intermediate 57: 5-tert-Butyl-2-methoxyphenol

The title compound was prepared from 2-bromo-5-tert-butylphenol (Intermediate 55, 2 g, 8.7 mmol) according to the procedure outlined in patent WO 02/098363. It was purified by column chromatography (ethyl acetate: petroleum ether, 1:9) and isolated as a white solid (0.97 g, 62%). ¹H NMR δ 7 (1H, m), 6.85-6.87 (1H, m), 6.78-6.8 (1H, m), 3.87 (3H, s) and 1.29 (9H, s). m/z 180.15 (MH+).

### Intermediate 58: tert-Butyl 2-(5-(2-(5-tert-butyl-2-methoxyphenoxy)thiazole-4-carboxamido)-4,6-dimethoxypyrimidin-2-ylamino)ethyl(isopropyl)carbamate

### 5-tert-butyl-2-methoxyphenol

The title compound was prepared according to the method outlined for Intermediate 1 from 5-tert-butyl-2-methoxyphenol (Intermediate 57, 0.23 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol). It was isolated as a pale yellow liquid (0.53 g, 90%); m/z 645.37 (MH+).

### Intermediate 59: 5-Bromo-2-methylphenol

To a solution of 5-amino-2-methylphenol (50 g, 0.41 mol) in hydrobromic acid (200 mL, 48% solution) and water (200 ml) in a brine ice bath was added sodium nitrite (30.5 g, 0.45 mol, 1.1 equiv.) portion-wise at such a rate that the temperature remained below +10° C., and the mixture was stirred for a further 15 minutes. To this solution was added copper(I) bromide (64 g, 0.44 mol, 1.1 equiv.) and the reaction was subsequently heated to 80° C. Effervescence was observed at 60-65° C. indicating that the reaction had occurred. The mixture was heated at 80° C. for a further 30 minutes and then allowed to cool to room temperature. The resulting black tarry mixture was extracted with petroleum ether 40-60° C. (4×400 ml) and the combined organic extracts were dried (MgSO₄) and concentrated under reduced pressure. [Note that a large tarry component does not dissolve in either petrol or the aqueous layer and sometimes the petrol layer was simply decanted from this once all the aqueous layer had been run out from the separating funnel]. The resulting orange-white solid was recrystallised from petrol to give the product as a white solid (13 g 17%). The mother liquors were further purified by dry flash column chromatography (particle size 3-35 A), eluting with 0-20% diethyl ether in petroleum ether 40-60° C. to give a yellow-white crystalline solid which was re-crystallised from petroleum ether 40-60° C. to give a further 5-g and an overall yield of 18 g (24%); R_{f}=0.26 (4:1 petrol-ethyl acetate), m.p. 77-78° C. (from petrol). ¹H NMR δ_{H} 2.21 (3H, s), 4.89-4.95 (1H, br, s), 6.96-6.97 (1H, br, m) and 6.99-7.00 (2H, m).

### Intermediate 60: tert-Butyl 2-(5-(2-(5-bromo-2-methylphenoxy)thiazole-4-carboxamido)-4,6-dimethoxypyrimidin-2-ylamino)ethyl(isopropyl)carbamate

The title compound was prepared according to the method outlined for Intermediate 1 from 5-bromo-2-methylphenol (Intermediate 59, 0.21 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol). It was isolated as a pale brown gum (0.35 g, 58%), m/z 653.22 (MH⁺).

### Intermediate 61: 4-Chloro-5-isopropyl-2-methylphenol

A couple of grains of iodine were added to 5-isopropyl-2-methylphenol (2 g, 13.3 mmol) dissolved in acetic acid (40 ml). Sulfuryl chloride (1.5 ml, 19.95 mmol) was added to the solution and the reaction was heated to 45° C. for 60 minutes. The reaction was cooled and then divided between dichloromethane (30 ml) and water (30 ml). The organic layer was washed with water until the aqueous layer was pH neutral and then dried (MgSO₄). The title compound was purified by column chromatography (ethyl acetate-petroleum ether, 1:19) and isolated as a pale yellow oil (1.37 g, 56%). ¹H NMR δ 7.14 (1H, s), 6.74 (1H, s), 2.23 (3H, s), 1.24 (3H, s) and 1.23 (3H, s); m/z 183.16 (MH⁻).

### Intermediate 62: tert-Butyl 2-(5-(2-(4-chloro-5-isopropyl-2-methylphenoxy)thiazole-4-carboxamido)-4,6-dimethoxypyrimidin-2-ylamino)ethyl(isopropyl)carbamate

The title compound was prepared from 4-chloro-5-isopropyl-2-methylphenol (Intermediate 61, 0.2 g, 1.1 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol) according to the method outlined for Intermediate 1. It was isolated as a pale brown gum (0.5 g, 100%); m/z 649.27 (MH+).

### Intermediate 63: tert-Butyl 2-(5-(2-(2-tert-butylphenoxy)thiazole-4-carboxamido)-4,6-dimethoxypyrimidin-2-ylamino)ethyl(isopropyl)carbamate

The title compound was prepared from 2-tert-butylphenol (0.18 g, 1.2 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol) according to the method outlined for Intermediate 1. It was isolated as a clear oil (0.26 g, 46%); m/z 615.22 (MH+).

### Intermediate 64: Propyl methanesulfonate

To a cooled (0° C.) mixture of propan-1-ol (2.9 ml, 38.4 mmol) and triethylamine (5.4 ml, 38.4 mmol) was added methanesulphonyl chloride (2.98 ml, 38.4 mmol). The reaction was stirred at 0° C. for 30 minutes and then for a further 30 minutes at ambient temperature before the dichloromethane was removed in vacuo and then the mixture was partitioned between ethyl acetate (20 ml) and water (20 ml). The organic layer was washed with brine (20 ml), dried (MgSO₄) and then the solvent was removed in vacuo to give the title compound as a yellow oil (5.3 g, 100%). ¹H NMR δ 4.19 (2H, t), 3.0 (3H, s), 1.74-1.82 (2H, m) and 1.0 (3H, t).

### Intermediate 65: 1-Methyl-2-nitro-4-propoxybenzene

To a solution of 4-methyl-3-nitrophenol (5 g, 32 mmol) in anhydrous THF (25 ml) was added potassium tert-butoxide (3.77 g, 33.6 mmol). The resulting suspension was stirred at ambient temperature for 60 minutes before a solution of propyl methanesulfonate (Intermediate 64, 5.3 g, 38.4 mmol) in DMSO (25 ml) was added and the whole stirred at 95° C. for 2 hours. The mixture was divided between water (40 ml) and ethyl acetate (40 ml) and the layers were separated before the organic layer was washed with aquoeus sodium hydroxide solution (2 N, 20 ml) and then washed with brine (20 ml). The organic layer was dried (MgSO₄) and the solvent was removed. The crude product was purified by column chromatography (SiO₂; 9:1 petroleum ether-ethyl acetate) to afford the title compound as a pale yellow oil (6.14 g, 98%). ¹H NMR δ 7.5 (1H, d), 7.21 (1H, m), 7.04-7.07 (1H, m), 3.94 (2H, t), 2.5 (3H, s), 1.78-1.87 (2H, m) and 1.05 (3H, t).

### Intermediate 66: Methyl-5-propoxybenzenamine

1-Methyl-2-nitro-4-propoxybenzene (Intermediate 65; 4.4 g, 22.6 mmol) was dissolved in ethanol (20 ml). The solution was flushed with nitrogen and then charged with palladium on charcoal (10% Pd on C, 500 mg) before the flask was evacuated and flushed with hydrogen. The suspension was stirred at ambient temperature for 5 h and then the reaction was flushed with nitrogen and filtered through a pad of filter aid. The volatiles were removed in vacuo to give the title compound as a pale brown oil (3.65 g, 98%). ¹H NMR δ 6.91-6.94 (1H, d), 6.26-6.29 (1H, m), 6.26 (1H, s), 3.86 (2H, t), 3.58 (2H, br s), 2.10 (3H, s), 1.73-1.82 (2H, m) and 1.01 (3H, t).

### Intermediate 67: 2-Methyl-5-propoxyphenol

2-Methyl-5-propoxybenzenamine (Intermediate 66; 1 g, 6.05 mmol) was dissolved in warm 15% sulphuric acid (40 ml) and the solution was cooled to 0° C. in an ice salt bath to form an off-white suspension of the salt. Sodium nitrite (0.46 g, 6.66 mmol) dissolved in water (5 ml) was added and the mixture was stirred at 0° C. for a further 30 minutes. Copper (II) nitrate (4.54 g, 24.21 mmol) dissolved in water (10 ml) was added followed immediately by the addition of copper (II) oxide. The reaction was stirred at 0° C. for a further 30 minutes and then at ambient temperature for 5 hours. The organic material was extracted into ethyl acetate (2×30 ml) and the resulting organic layer was washed with brine (40 ml), dried (MgSO₄) and the solvent was removed in vacuo. The crude product was then purified by column chromatography (SiO₂; 9:1 petroleum ether-ethyl acetate) to afford the title compound as a pale yellow oil (0.34 g, 34%). ¹H NMR δ 6.98-7.0 (1H, m), 6.4-6.43 (1H, m), 6.38-6.39 (1H, m), 4.67 (3H, s), 3.87 (3H, t), 2.17 (3H, s), 1.74-1.83 (2H, m), and 1.02 (3H, t).

### Intermediate 68: tert-Butyl 2-(4,6-dimethoxy-5-(2-(2-methyl-5-propoxyphenoxy)thiazole-4-carboxamido)pyrimidin-2-ylamino)ethyl(isopropyl)carbamate

The title compound was prepared from 2-methyl-5-propoxyphenol (Intermediate 67; 0.31 g, 1.87 mmol) and tert-butyl 2-(5-(2-bromothiazole-4-carboxamido)-4,6-dimethoxypyrimidin-2-ylamino)ethyl(isopropyl)carbamate (Intermediate 45; 0.5 g, 0.92 mmol) according to the method outlined for Intermediate 1. It was isolated as a clear oil (0.35 g, 60%); m/z 631.22 (MH+).

### Intermediate 69: Isopropyl methanesulfonate

The title compound was prepared from propan-2-ol (2.94 ml, 38.4 mmol) according to the procedure outlined for propyl methanesulfonate (Intermediate 64). The compound was isolated as a yellow oil (5.3 g, 100%). ¹H NMR δ 4.91-4.97 (1H, m), 2.99 (3H, s), 1.42 (3H, s) and 1.4 (3H, s).

### Intermediate 70: 4-Isopropoxy-1-methyl-2-nitrobenzene

The title compound was prepared from 4-methyl-3-nitrophenol (5 g, 32 mmol) and isopropyl methanesulfonate (Intermediate 69, 5.3 g, 38.4 mmol) according to the method outlined for 1-methyl-2-nitro-4-propoxybenzene (Intermediate 65). It was isolated as a pale yellow oil (5.11 g, 82%). ¹H NMR δ7.5 (1H, d), 7.2 (1H, m), 7.02-7.05 (1H, m), 4.55-4.6 (1H, m), 2.5 (3H, s), 1.36 (3H, s) and 1.35 (3H, s).

### Intermediate 71: 5-Isopropoxy-2-methylbenzenamine

The title compound was prepared from 4-isopropoxy-1-methyl-2-nitrobenzene (Intermediate 70, 3.92 g, 20 mmol) according to the method outlined for 2-methyl-5-propoxybenzenamine (Intermediate 66) and isolated as a pale brown oil (3.3 g, 100%). ¹H NMR δ 6.91-6.93 (1H, d), 6.26-6.28 (1H, m), 6.26 (1H, s), 4.43-4.49 (1H, m), 3.57 (2, br s), 2.10 (3H, s), 1.31 (3H, s), and 1.30 (3H, s).

### Intermediate 72: 5-Isopropoxy-2-methylphenol

5-Isopropoxy-2-methylbenzenamine (Intermediate 71; 2.3 g, 13.92 mmol) was dissolved in warm 15% sulphuric acid (100 ml) and the mixture was cooled to 0° C. in an ice salt bath to form an off-white suspension of the salt. Sodium nitrite (0.96 g, 13.92 mmol) dissolved in water (5 ml) was added and the mixture was stirred at 0° C. for 30 minutes. The resulting yellow suspension was warmed for 20 minutes (50° C.) and the organic material extracted in to ethyl acetate (2×30 ml). The organic layer was washed with brine (40 ml), dried (MgSO₄) and the solvent was removed in vacuo. The crude product was purified by column chromatography (SiO₂; 9:1 petroleum ether-ethyl acetate) to afford the title compound as a pale yellow oil (1.3 g, 57%). ¹H NMR δ 7.0-7.02 (1H, d), 6.42-6.45 (1H, m), 6.39-6.4 (1H, m), 4.64 (1H, s), 4.46-4.52 (1H, m), 2.20 (3H, s), 1.34 (3H, s) and 1.33 (3H, s).

### Intermediate 73: tert-Butyl 2-(5-(2-(5-isopropoxy-2-methylphenoxy)thiazole-4-carboxamido)-4,6-dimethoxypyrimidin-2-ylamino)ethyl(isopropyl)carbamate

The title compound was prepared from 5-isopropoxy-2-methylphenol (Intermediate 72; 0.32 g, 1.9 mmol) and tert-butyl 2-(5-(2-bromothiazole-4-carboxamido)-4,6-dimethoxypyrimidin-2-ylamino)ethyl(isopropyl)carbamate (Intermediate 45; 0.8 g, 1.46 mmol) according to the method outlined for Intermediate 1. It was isolated as a dark yellow oil (0.59 g, 64%); m/z 631.16 (MH+).

### Intermediate 74: Ethyl methanesulfonate

Prepared from ethanol (2.43 ml, 41.6 mmol) according to the procedure outlined for propyl methanesulfonate (Intermediate 64). The title compound was isolated as a yellow oil (5.1 g, 100%). ¹H NMR δ 4.28-4.33 (2H, q), 3.0 (3H, s) and 1.42 (3H, t).

### Intermediate 75: 4-Ethoxy-1-methyl-2-nitrobenzene

The title compound was prepared from 4-methyl-3-nitrophenol (5 g, 32 mmol) and ethyl methanesulfonate (Intermediate 74, 5.1 g, 41.6 mmol) according to the method outlined for 1-methyl-2-nitro-4-propoxybenzene (Intermediate 65). It was isolated as a pale yellow oil (5.59 g, 96%). ¹H NMR δ 7.49-7.5 (1H, d), 7.2-7.23 (1H, m), 7.03-7.06 (1H, m), 4.06 (2H, q), 2.52 (3H, s) and 1.43 (3H, t).

### Intermediate 76: 5-Ethoxy-2-methylbenzenamine

The title compound was prepared from 4-ethoxy-1-methyl-2-nitrobenzene (Intermediate 75, 4.43 g, 24.4 mmol) according to the method outlined for 2-methyl-5-propoxybenzenamine (Intermediate 66) and isolated as a pale brown oil (3.5 g, 95%). ¹H NMR δ 6.92-6.94 (1H, d), 6.26-6.29 (1H, m), 6.26 (1H, s), 3.95-4.0 (2H, m), 3.58 (2H, br s), 2.1 (3H, s), and1.38 (3H, t).

### Intermediate 77: 5-Ethoxy-2-methylphenol

The title compound was prepared from 5-ethoxy-2-methylbenzenamine (Intermediate 76; 2 g, 13.22 mmol) according to the method outlined for 5-isopropoxy-2-methylphenol (Intermediate 72). It was isolated as a pale grey liquid (1.2 g, 60%). ¹H NMR δ 6.98-7.0 (1H, m), 6.40-6.43 (1H, m), 6.38-6.39 (1H, m), 3.95-4.0 (2H, m), 2.17 (3H, s) and 1.39 (3H, t).

### Intermediate 78: tert-Butyl 2-(5-(2-(5-ethoxy-2-methylphenoxy)thiazole-4-carboxamido)-4,6-dimethoxypyrimidin-2-ylamino)ethyl(isopropyl)carbamate

The title compound was prepared from 5-ethoxy-2-methylphenol (Intermediate 77; 0.22 g, 1.43 mmol) and tert-butyl 2-(5-(2-bromothiazole-4-carboxamido)-4,6-dimethoxypyrimidin-2-ylamino)ethyl(isopropyl)carbamate (Intermediate 45; 0.6 g, 1.1 mmol) according to the method outlined for Intermediate 1. It was isolated as an off-white foam (0.57 g, 64%); m/z 617.14 (MH⁺).

### Intermediate 79: Isobutyl methanesulfonate

Prepared from 2-methylpropan-1-ol (3.85 ml, 41.6 mmol) according to the procedure outlined for propyl methanesulfonate (Intermediate 64). The title compound was isolated as a yellow oil (6.1 g, 97%). ¹H NMR δ 3.99-4 (2H, d), 3.0 (3H, s), 1.99-2.1 (1H, m), 1.0 (3H, s) and 0.98 (3H, s).

### Intermediate 80: 4-Isobutoxy-1-methyl-2-nitrobenzene

The title compound was prepared from 4-methyl-3-nitrophenol (5 g, 32 mmol) and isobutyl methanesulfonate (Intermediate 79, 6.1 g, 40.35 mmol) according to the method outlined for 1-methyl-2-nitro-4-propoxybenzene (Intermediate 65). It was isolated as a pale yellow liquid (6.69 g, 100%). ¹H NMR δ 7.49-7.5 (1H, d), 7.2-7.22 (1H, m), 7.04-7.07 (1H, m), 3.73-3.75 (2H, d), 2.52 (3H, S), 2.06-2.13 (1H, m), 1.04 (3H, s) and 1.02 (3H, s).

### Intermediate 81: 5-Isobutoxy-2-methylbenzenamine

The title compound was prepared from 4-isobutoxy-1-methyl-2-nitrobenzene (Intermediate 80, 4.53 g, 21.6 mmol) according to the method outlined for 2-methyl-5-propoxybenzenamine (Intermediate 66) and isolated as a pale brown oil (3.8 g, 98%). ¹H NMR δ 6.91-6.93 (1H, m), 6.26-6.29 (1H, m), 6.26 (1H, s), 3.65-3.67 (2H, d), 3.57 (2H, br s), 2.10 (3H, s), 2.0-2.1 (1H, m), 1.0 (3H, s) and 0.99 (3H, s).

### Intermediate 82: 5-Isobutoxy-2-methyl henol

The title compound was prepared from 5-isobutoxy-2-methylbenzenamine (Intermediate 81; 2.5 g, 13.96 mmol) according to the method outlined for 5-isopropoxy-2-methylphenol (Intermediate 72). It was isolated as a pale grey liquid (1.52 g, 61%). ¹H NMR δ 6.97-6.99 (1H, m), 6.40-6.42 (1H, m), 6.38-6.39 (1H, m), 3.65-3.67 (2H, d), 2.17 (3H, s) 2.02-2.09 (1H, m), 1.0 (3H, s). and 0.99 (3H, s).

### Intermediate 83: tert-Butyl 2-(5-(2-(5-isobutoxy-2-methylphenoxy)thiazole-4-carboxamido)-4,6-dimethoxypyrimidin-2-ylamino)ethyl(isopropyl)carbamate

The title compound was prepared according to the method outlined for Intermediate 1 from 5-isobutoxy-2-methylphenol (Intermediate 82; 0.26 g, 1.43 mmol) and tert-butyl 2-(5-(2-bromothiazole-4-carboxamido)-4,6-dimethoxypyrimidin-2-ylamino)ethyl(isopropyl)carbamate (Intermediate 45; 0.6 g, 1.1 mmol). It was isolated as a dark yellow oil (0.55 g, 77%); m/z 645.2 (MH⁺).

### Intermediate 84: 4-(2-(4-Methyl-3-nitrophenoxy)ethyl)morpholine

To a solution of 4-methyl-3-nitrophenol (5 g, 32 mmol) in anhydrous THF (20 ml) was added potassium tert-butoxide (3.77 g, 33.6 mmol). The resulting mixture was stirred at ambient temperature for 60 minutes before a solution of 4-(2-chloroethyl)morpholine hydrochloride (6 g, 3 mmol) in DMSO (20 ml) was added followed by tetrabutylammonium iodide (11.8 g, 32 mmol). The resulting mixture was stirred at 95° C. for 2 hours before being partitioned between water (40 ml) and ethyl acetate (40 ml). The layers were separated and then the organic layer was washed with aqueous sodium hydrorxide solution (20 ml) and then washed with brine (20 ml). The organic layer was dried (MgSO₄) and the solvent was removed before the crude product was purified by column chromatography (SiO₂; 1:1 petroleum ether: ethyl acetate) to afford the title compound as a white solid (5.2 g, 61%); m/z 267.11 (MH⁺).

### Intermediate 85: 2-Methyl-5-(2-morpholinoethoxy)benzenamine

The title compound was prepared from 4-(2-(4-methyl-3-nitrophenoxy)ethyl)morpholine (Intermediate 84; 4.53 g, 21.6 mmol) according to the method outlined for 2-methyl-5-propoxybenzenamine (Intermediate 66). The crude product was then purified by column chromatography (SiO₂; 1:9 methanol:dichloromethane) to afford the product as a pale brown solid (4.6 g, 100%). ¹H NMR δ 6.91-6.93 (1H, m), 6.25-6.28 (2H, m), 4.05 (2H, t), 3.72-3.74 (4H, m), 3.59 (2H, br s), 2.77 (2H, t), 2.55-2.57 (4H, m) and 2.09 (3H, s); m/z 237.27 (MH+).

### Intermediate 86: 2-Methyl-5-(2-morpholinoethoxy)phenol

The title compound was prepared from 2-methyl-5-(2-morpholinoethoxy)benzenamine (Intermediate 85; 1 g, 4.24 mmol) according to the method outlined for 5-isopropoxy-2-methylphenol (Intermediate 72). It was isolated as an off-white solid (0.3 g, 30%). ¹H NMR δ 6.98-7.0 (1H, m), 6.38-6.42 (2H, m), 4.07 (2H, t), 3.73-3.75 (4H, m) 2.78 (2H, t), 2.57-2.59 (4H, m) and 2.17 (3H, s); m/z 238.23 (MH+).

### Intermediate 87: N-(2-(2-(tert-Butyldimethylsllyloxy)ethylamino)-4,6-dimethoxypyrimidin-5-yl)-2-(2-methyl-5-(2-morpholinoethoxy)phenoxy)thiazole-4-carboxamide

The title compound was prepared from 2-methyl-5-(2-morpholinoethoxy)phenol (Intermediate 86; 0.3 g, 1.26 mmol) and N-(2-(2-(tert-butyldimethylsilyloxy)ethylamino)-4,6-dimethoxypyrimidin-5-yl)-2-chlorothiazole-4-carboxamide (Intermediate 36; 0.50 g, 1.05 mmol) according to the method outlined for Intermediate 1. The compound was isolated as a pale brown oil (0.52 g, 73%); m/z 675.26 (MH⁺).

### Intermediate 88: 2-[(4,6-Dimethoxy-5-nitro-pyrimidin-2-ylamino)-methyl]-pyrrolidine-1-carboxylic acid tert-butyl ester

2-Chloro-4,6-dimethoxy-5-nitro-pyrimidine (Intermediate 13; 0.5 g, 2.29 mmol) was taken up in absolute ethanol (25 ml) and treated with triethylamine (0.352 ml, 2.08 mmol), cooled in an ice-water bath and then 2-aminomethyl-pyrrolidine-1-carboxylic acid tert-butyl ester (0.506 g, 2.08 mmol) was added. The cooling bath was allowed to warm to ambient temperature and stirring continued overnight. The reaction mixture was evaporated in vacuo and the residue was divided between saturated sodium hydrogen carbonate solution (25 ml) and ethyl acetate (25 ml). The layers were separated and the organic portion was dried (MgSO₄) and the solvent was removed in vacuo to afford the title compound as a tan oil (1.35 g, 100%)

¹H NMR δ 4.49 (2H, t), 4.06 (6H, s), 3.58-3.45 (3H, br. m), 3.43-3.36 (2H, br m), 1.92 (2H, br s), 1.78 (2H, br s) and 1.49 (9H, m). m/z 382.26 (M−H—).

### Intermediate 89: 2-[(5-Amino-4,6-dimethoxy-pyrimidin-2-ylamino)-methyl]-pyrrolidine-1-carboxylic acid tert-butyl ester

2-[(4,6-Dimethoxy-5-nitro-pyrimidin-2-ylamino)-methyl]-pyrrolidine-1-carboxylic acid tert-butyl ester (Intermediate 88; 1.35 g, 3.52 mmol) was dissolved in absolute ethanol (25 ml). The solution was flushed with nitrogen and then charged with palladium on charcoal (10% Pd on C, 50% water, 200 mg) before the flask was evacuated and flushed with hydrogen. The suspension was stirred at ambient temperature for 18 h and then the reaction was flushed with nitrogen and filtered through a pad of filter aid. The volatiles were removed in vacuo to give the title compound as a pale brown oil (1.23 g, 99%); m/z 354.12 (MH⁺)

### Intermediate 90: 4-(4.6 Dimethoxy-5-nitro-pyrimidin-2-yl)-[1,4]diazepane-1-carboxylic acid tert-butyl ester

2-Chloro-4,6-dimethoxy-5-nitro-pyrimidine (Intermediate 13; 0.5 g, 2.29 mmol) was taken up in absolute ethanol (25 ml) and treated with triethylamine (0.352 ml, 2.08 mmol), cooled in an ice-water bath and then [1,4]Diazepane-1-carboxylic acid tert-butyl ester (0.506 g, 2.08 mmol) was added in one portion. The cooling bath was allowed to warm to ambient temperature and stirring continued overnight. The reaction mixture was evaporated in vacuo and the residue was divided between saturated aqueous sodium hydrogen carbonate solution (25 ml) and ethyl acetate (25 ml). The layers were separated and the organic layer was dried (MgSO₄) and the solvent was removed in vacuo to afford the title compound as a tan oil (0.862 g, 100%) ¹H NMR δ 4.01 (6H, s), 3.91 (2H, m), 3.79 (2H, t), 3.58 (2H, m), 3.41 (2H, t), 3.33 (2H, t), 1.99 (2H, t) and 1.46 (9H, d).

### Intermediate 91: 4-(5-Amino-4,6-dimethoxy-pyrimidin-2-yl)-[1,4]diazepane-1-carboxylic acid tert-butyl ester

4-(4,6 Dimethoxy-5-nitro-pyrimidin-2-yl)-[1,4]diazepane-1-carboxylic acid tert-butyl ester (Intermediate 90; 0.859 g, 2.24 mmol) was dissolved in absolute ethanol (25 ml). The solution was flushed with nitrogen and then charged with palladium on charcoal (10% Pd on C, 50% water, 200 mg) before the flask was evacuated and flushed with hydrogen. The suspension was stirred at ambient temperature for 18 h and then the reaction was flushed with nitrogen and filtered through a pad of filter aid. The volatiles were removed in vacuo to give the title compound as a pale brown oil (0.817 g, 100%).

### Intermediate 92: 4-(4,6-Dimethoxy-5-nitro-pyrimidin-2-yl)-piperazine-1-carboxylic acid tert-butyl ester

2-Chloro-4,6-dimethoxy-5-nitro-pyrimidine (Intermediate 13; 0.5 g, 2.29 mmol) was taken up in absolute ethanol (25 ml) and treated with triethylamine (0.352 ml, 2.08 mmol), cooled in an ice-water bath and then piperazine-1-carboxylic acid tert-butyl ester (0.470 g, 2.08 mmol) was added in one portion. The cooling bath was allowed to warm to ambient temperature and stirring continued overnight. The reaction mixture was evaporated in vacuo and the residue was divided between saturated aqueous sodium hydrogen carbonate solution (25 ml) and ethyl acetate (25 ml). The layers were separated and the organic layer was dried (MgSO₄) and the solvent was removed in vacuo to afford the title compound as a tan oil (0.903 g, 100%). ¹H NMR δ 4.02 (6H, s), 3.87 (4H, m), 3.53 (4H, m) and 1.51 (9H, s).

### Intermediate 93: 4-(5-Amino-4,6-dimethoxy-pyrimidin-2-yl)-piperazine-1-carboxylic acid tert-butyl ester

4-(4,6-Dimethoxy-5-nitro-pyrimidin-2-yl)-piperazine-1-carboxylic acid tert-butyl ester (Intermediate 92; 0.900 mg, 2.4 mmol) was dissolved in absolute ethanol (25 ml). The solution was flushed with nitrogen and then charged with palladium on charcoal (10% Pd on C, 50% water, 200 mg) before the flask was evacuated and flushed with hydrogen. The suspension was stirred at ambient temperature for 18 h and then the reaction was flushed with nitrogen and filtered through a pad of filter aid. The volatiles were removed in vacuo to give the title compound as a pale brown oil (0.878 g, Quant).

### Intermediate 94: 2-Bromo-N-(2-(2-(isopropylamino)ethylamino)-4,6-dimethoxypyrimidin-5-yl)thiazole-4-carboxamide

tert-Butyl 2-(5-(2-bromothiazole-4-carboxamido)-4,6-dimethoxypyrimidin-2-ylamino)ethyl(isopropyl)carbamate (Intermediate 45; 0.545 g, 1 mmol) was dissolved in a mixture of dichloromethane (5 ml) and trifluoroacetic acid (5 ml) and the reaction was stirred for one hour. The reaction mixture was evaporated and the residue was partitioned between ethyl acetate and sodium carbonate solution. The organic extract was dried (MgSO₄) and the solvent removed in vacuo to yield the title compound as a pale brown solid (0.41 g, 92%); m/z 445.01 (M⁺) and 446.99 (M+H⁺).

### Intermediate 95: Ethyl 5-bromo-4-methyl-4H-1,2,4-triazole-3-carboxylate and ethyl 5-bromo-2-methyl-2H-1,2,4-triazole-3-carboxylate and ethyl 5-bromo-1-methyl-1H-1,2,4-triazole-3-carboxylate

Ethyl 5-bromo-2H-1,2,4-triazole-3-carboxylate (Hechung Huaxue, 2004, 12(2), 191-193, Chem. Abs. 141:350095) (3.11 g, 0.14 mmol) was dissolved in methanol (50 ml) and cooled to 0° C. Trimethylsilyl diazomethane (˜14 ml of a 2 M solution in ether) was added until a pale yellow colour persisted and the reaction was warmed to room temperature and quenched by the addition of excess acetic acid. It was then evaporated and the residue was partitioned between ethyl acetate and saturated sodium bicarbonate. The organic extract was dried, filtered and evaporated to yield a residue which was purified and separated into its three isomers by silica chromatography using ethyl acetate/petrol.

Isomer A (least polar) (1.2 g, 5.1 mmol). ¹H NMR (DMSO) δ 4.36 (2H, q, J=7 Hz), 4.10 (3H, s), 1.32 (3H, t, J=7 Hz)

Isomer B (medium polarity) (0.47 g, 2.0 mmol). ¹H NMR (DMSO) δ 4.31 (2H, q, J=7 Hz), 3.92 (3H, s), 1.29 (3H, t, J=7 Hz)

Isomer C (most polar) (0.37 g, 1.6 mmol). ¹H NMR (DMSO) 84.38 (2H, q, J=7 Hz), 3.82 (3H, s), 1.33 (3H, t, J=7 Hz)

Combined yield 62%.

### Intermediate 96: Ethyl 5-(3-tert-butylphenoxy)-4-methyl-4H-1,2,4-triazole-3-carboxylate or Ethyl 5-(3-tert-butylphenoxy)-1-methyl-1H-1,2,4-triazole-3-carboxylate or Ethyl 5-(3-tert-butylphenoxy)-2-methyl-2H-1,2,4-triazole-3-carboxylate

The title compound was prepared according to the method outlined for Intermediate 1 from 3-tert-butyl phenol (0.450 g, 3 mmol) and ethyl 5-bromo-4-methyl-4H-1,2,4-triazole-3-carboxylate or ethyl 5-bromo-2-methyl-2H-1,2,4-triazole-3-carboxylate or ethyl 5-bromo-1-methyl-1H-1,2,4-triazole-3-carboxylate Intermediate 95 (Isomer B); (0.47, 2 mmol) to yield the title compound (0.345 g, 57%). ¹H NMR (d⁶-DMSO) δ 7.34-7.30 (1H, m), 7.26-7.24 (2H, m), 7.15-7.12 (1H, m), 4.3 (2H, q, J=7 Hz), 3.87 (3H, s), 1.39 (3H, t, J=7 Hz) and 1.32 (9H, s).

### Intermediate 97: 5-tert-butyl-2-chlorophenol

5-tert-butyl-2-chlorobenzenamine (17 g, 93 mmol) was dissolved in a mixture of conc. sulfuric acid (20 ml) and water (15 ml) and the solution was cooled to +5° C. Sodium nitrite (7.1 g, 102 mmol, 1.1 eq.) dissolved in water (8 ml) was added drop-wise at such a rate that the temperature of the mixture did not rise above +10° C. and the reaction was stirred at <10° C. for a further 20 minutes. The resulting mixture was then added drop-wise to a vigorously stirred solution of conc. sulfuric acid (55 ml) and water (55 ml) at 60° C. and then heated to 110° C. for 10 minutes. The solution was cooled and water (400 ml) and ethyl acetate (400 ml) were added and the layers were separated. The aqueous layer was then extracted with ethyl acetate (2×200 ml) and the combined organic layers were dried (MgSO₄) and the solvent was removed under reduced pressure. The crude product was then purified by dry flash column chromatography (SiO₂; 0-30% ethyl acetate in petrol) to afford the title compound as a pale yellow oil (12 g, 70%). MS (ES⁺) m/z 185 (MH⁺).

### Intermediate 98: tert-Butyl 2-(5-(2-(5-tert-butyl-2-chlorophenoxy)thiazole-4-carboxamido)-4,6-dimethoxypyrimidin-2-ylamino)ethyl(isopropyl)carbamate

5-tert-Butyl-2-chlorophenol (intermediate 97, 100 mg, 0.55 mmol, 1.5 eq.) was dissolved in THF (4 ml) and potassium tert-butoxide (62 mg, 0.55 mmol, 1.5 eq.) was added. The resulting solution was heated at 70° C. under nitrogen for 1 hour, after which time the reaction mixture was cooled to 50° C. and tert-butyl 2-(5-(2-bromothiazole-4-carboxamido)-4,6-dimethoxypyrimidin-2-ylamino)ethyl(isopropyl)carbamate (intermediate 45, 200 mg, 0.37 mmol) was added as a solution in DMSO (2 ml). This mixture was heated at 85° C. for 24 h, after which time the THF was removed under reduced pressure and dichloromethane (10 ml) and saturated aqueous sodium bicarbonate solution (5 ml) were added. The layers were separated and the aqueous layer was extracted with dichloromethane (10 ml). The combined organic layers were dried (MgSO₄) and the solvent was removed under reduced pressure. The crude product was then purified by column chromatography (silica; 0-10% methanol in dichloromethane) to afford the title compound as a brown oil (249 mg, 70%). ¹H NMR (d⁶-DMSO) δ 1.15 (6H, d), 1.31 (9H, s), 1.44 (9H, s), 3.30 (2H, m), 3.51 (H, q), 3.90 (6H, br s), 7.28 (1H, d), 7.44 (1H, d), 7.71 (1H, s), 7.89 (1, s); MS (ES⁺) m/z 648 (M⁺).

### Intermediate 99: tert-Butyl 2-((4,6-dimethoxy-5-nitropyrimidin-2-ylamino)methyl)morpholine-4-carboxylate

To a mixture of 2-chloro-4,6-dimethoxy-5-nitropyrimidine (intermediate 13; 1 g, 4.6 mmol), Boc-2-(aminomethyl)morpholine-4-carboxylate and ethanol was added triethylamine (0.77 ml, 5.5 mmol, 1.2 eq.) and the solution was heated under reflux for 4 hours. After this time the ethanol was removed under reduced pressure and dichloromethane (100 ml) and saturated aqueous sodium bicarbonate solution (50 ml) were added. The layers were separated and the aqueous layer was extracted with dichloromethane (50 ml). The combined organic layers were dried (MgSO₄) and the solvent was removed under reduced pressure. The crude product was then purified by column chromatography (silica, 0-15% methanol-dichloromethane) to afford the title compound as a yellow oil (100 mg, 5.5%); MS (ES⁺) m/z 398 (MH⁺).

### Intermediate 100: tert-Butyl 2-((5-amino-4,6-dimethoxypyrimidin-2-ylamino)methyl)morpholine-4-carboxylate

tert-Butyl 2-((4,6-dimethoxy-5-nitropyrimidin-2-ylamino)methyl)morpholine-4-carboxylate (intermediate 99; 100 mg, 0.25 mmol) was dissolved in ethanol (5 ml). The solution was flushed with nitrogen and then charged with palladium on charcoal (10% Pd/C, wet, 10 mg) before the flask was evacuated and flushed with hydrogen. The suspension was stirred at ambient temperature for 24 h and then the product mixture was filtered through a pad of celite. The ethanol was removed under reduced pressure to give the title compound as a pale brown oil (84 mg, 91%).

### Intermediate 101: tert-Butyl 2-((5-tert-butyl-2-methylphenoxy)thiazole-4-carboxamido)-4,6-dimethoxypyrimidin-2-ylamino)methyl)morpholine-4-carboxylate

2-(5-tert-butyl-2-methylphenoxy)thiazole-4-carboxylic acid (intermediate 18, 71 mg, 0.24 mmol, 1.1 eq.) and tert-butyl 2-((5-amino-4,6-dimethoxypyrimidin-2-ylamino)methyl)morpholine-4-carboxylate (Intermediate 100, 82 mg, 0.22 mmol, 1.2 eq.) were dissolved in dichloromethane (3 ml) and HBTU (109 mg, 0.29 mmol, 1.2 eq.) and triethylamine (0.07 ml, 0.48 mmol, 2 eq.) were added and the solution was stirred at room temperature overnight. Saturated aqueous sodium bicarbonate solution (2 ml) was added. The layers were separated and the aqueous layer was extracted with dichloromethane (5 ml). The combined organic layers were dried (MgSO₄) and the solvent was removed under reduced pressure. The crude product was then purified by column chromatography (silica, 0-15% methanol-dichloromethane) to give the product as a brown oil (130 mg, 92%); m/z 642 (MH⁺).

### Intermediate 102: 1-((2-(Trimethylsilyl)ethoxy)methyl)-1H-imidazole

Under a blanket of nitrogen, sodium hydride (6.8 g, 0.17 mol, 60% dispersion in mineral oil) was added portion-wise to a solution of imidazole (11.9 g, 0.175 mol) dissolved in dimethylformamide (200 ml). The mixture was stirred at ambient temperature (2 h) before adding neat (2-(chloromethoxy)ethyl)trimethylsilane dropwise (30.8 g, 0.185 mol). The solution was stirred at ambient temperature for 2 hours and then quenched with water (200 ml). The organic material was then extracted into ethyl acetate (4×200 ml), washed with water (20 ml), brine (200 ml) and dried (MgSO₄) prior to concentration in vacuo. The crude product was then purified by column chromatography (silica; methanol-dichloromethane 1-19) to afford the title compound as a pale yellow oil (27 g, 79%). ¹H NMR (d⁶-DMSO) δ 7.57 (1H, s), 7.08 (1H, s), 7.02 (1H, s), 5.25 (2H, s), 3.45 (2H, t), 0.85-0.9 (2H, m) and 0 (9H, s).

### Intermediate 103: 1-((2-(trimethylsilyl)ethoxy)methyl)-1H-imidazole-2-carbaldehyde

n-Butyl lithium (59 ml, 1.6 M solution in hexane, 94 mmol) was added portionwise over 60 minutes to a cooled (−70° C.) solution of 1-((2-(trimethylsilyl)ethoxy)methyl)-1H-imidazole (Intermediate 102, 18.7 g, 94 mmol) in anhydrous THF (150 ml). Anhydrous DMF (8.4 ml, 94 mmol) was added to the cooled (−70° C.) mixture and then it was allowed to return to ambient temperature and stirred for 16 hours. Saturated ammonium chloride (100 ml) was added portionwise over 20 minutes to quench the reaction. The lithium salts were filtered off and the filtrate washed with ethyl acetate (2×200 ml). The organic layer was washed with brine, dried (MgSO₄) and then concentrated in vacuo. The crude product was then purified by column chromatography (silica; methanol-dichloromethane 1-19) to afford the title compound as a pale yellow liquid (10.6 g, 50%). ¹H NMR δ 9.83 (1H, s), 7.36 (1H, s), 7.33 (1H, s), 5.78 (2H, s), 3.53-3.58 (2H, m), 0.89-0.98 (2H, m) and 0 (9H, s).

### Intermediate 104: (1-((2-(trimethylsilyl)ethoxy)methyl)-1H-imidazol-2-yl)methanol

Sodium borohydride (1.78 g, 47 mmol) was added portion-wise to a solution of 1-((2-(trimethylsilyl)ethoxy)methyl)-1H-imidazole-2-carbaldehyde (Intermediate 103, 10.6 g, 47 mmol) in ethanol (60 ml) and stirred at ambient temperature (1 h). The material was divided between ethyl acetate (100 ml) and water (100 ml). The organic layer was washed with brine (100 ml), dried (MgSO₄) and then the solvent removed in vacuo. The crude product was then purified by column chromatography (SiO₂; methanol-dichloromethane 1-19) to afford the title compound as a white solid (8.9 g, 83%). ¹H NMR δ 6.99 (1H, s), 6.96 (1H, s), 5.34 (2H, s), 4.73 (2H, s), 3.5-3.54 (2H, m), 0.9-0.93 (2H, m) and 0 (9H, s).

### Intermediate 105: 2-((1-((2-(trimethylsilyl)ethoxy)methyl)-1H-imidazol-2-yl)methyl)isoindoline-1,3-dione

Diisopropylazadicarboxylate (1.7 ml, 8.66 mmol) was added to a mixture of phthalimide (1.27 g, 8.66 mmol), triphenylphosphine (2.27 g, 8.66 mmol) and (1-((2-(trimethylsilyl)ethoxy)methyl)-1H-imidazol-2-yl)methanol (Intermediate 104, 1.88 g, 8.25 mmol) in THF (30 ml) and stirred at ambient temperature for 2 hours. The solvent was removed in vacuo and the crude product was then purified by column chromatography (SiO₂; ethyl acetate-petrol 1:1) to afford the title compound as a white solid (1.5 g, 51%); m/z 358.08 (MH⁺).

### Intermediate 106: (1-((2-(trimethylsilyl)ethoxy)methyl)-1H-imidazol-2-yl)methanamine

Hydrazine hydrate (0.3 ml, 5.9 mmol) was added to a solution of 2-((1-((2-(trimethylsilyl)ethoxy)methyl)-1H-imidazol-2-yl)methyl)isoindoline-1,3-dione (Intermediate 105, 1.41 g, 3.95 mmol) in ethanol (30 ml) and heated to 60° C. for 1 hour. The resulting mixture was allowed to cool to room temperature before the precipitate was removed by filtration the solvent reduced to dryness in vacuo. The material was dissolved in ethyl acetate (50 ml) and extracted in to dilute hydrochloric acid (2 M, 50 ml). The aqueous layer was basified to pH 14 with sodium hydroxide solution (2 M) and then extracted in to ethyl acetate. The organic layer was washed with brine, dry (MgSO₄) and the solvent removed in vacuo. The title compound was isolated as a yellow oil (460 mg, 51%). ¹H NMR δ 6.96 (1H, s), 6.95 (1H, s), 5.29 (2H, s), 4.73 (2H, s), 3.96 (2H, s), 3.47-3.52 (2H, m), 1.63 (2H, br s) 0.88-0.92 (2H, m) and 0.00 (9H, s).

### Intermediate 107: 4,6-Dimethoxy-5-nitro-N-((1-(2-(trimethylsilyl)-ethoxy)methyl)-1H-imidazol-2-yl)methyl)pyrimidin-2-amine

(1-((2-(trimethylsilyl)ethoxy)methyl)-1H-imidazol-2-yl)methanamine (Intermediate 106, 445 mg, 1.96 mmol) was added to a solution of 2-chloro-4,6-dimethoxy-5-nitropyrimidine (Intermediate 13, 0.32 g, 1.46 mmol) and triethylamine (0.2 ml, 1.46 mmol) in ethanol (10 ml). The solution was stirred at ambient temperature (2 h), the solvent was removed in vacuo and the crude product was then purified by column chromatography (silica; methanol-dichloromethane 1-19) to afford the title compound as a yellow liquid (0.55 g, 92%); m/z 411.10 (MH⁺).

### Intermediate 108: 4,6-Dimethoxy-N2-((1-((2-(trimethyisilyl)ethoxy)-methyl)-1H-imidazol-2-yl)methyl)pyrimidine-2,5-diamine

The title compound was prepared from 4,6-dimethoxy-5-nitro-N-((1-((2-(trimethylsilyl)ethoxy)methyl)-1H-imidazol-2-yl)methyl)pyrimidin-2-amine (Intermediate 107, 0.55 g, 1.34 mmol) by the same procedure as outlined for 2-(2-tert-butoxyethoxy)-4,6-dimethoxypyrimidin-5-amine (Intermediate 35). The title compound was isolated as a pale brown oil (494 mg, 96%); m/z 381.1 (MH⁺).

### Intermediate 109: 2-Bromo-N-(4,6-dimethoxy-2-((1-((2-(trimethylsilyl)ethoxy)methyl)-1H-imidazol-2-yl)methylamino)pyrimidin-5-yl)thiazole-4-carboxamide

To a mixture of 4,6-dimethoxy-N2-((1-((2-(trimethylsilyl)ethoxy)methyl)-1H-imidazol-2-yl)methyl)pyrimidine-2,5-diamine (Intermediate 108, 494 mg, 1.3 mmol), 2-bromothiazole-4-carboxylic acid (Intermediate 41, 272 mg, 1.3 mmol) and triethylamine (0.37 ml, 2.6 mmol) in dichloromethane (20 ml) was added HBTU (0.5 g, 1.3 mmol). The reaction was stirred at ambient temperature for 18 h. The solvent was removed in vacuo and then the mixture divided between ethyl acetate (50 ml) and dilute hydrochloric acid (0.1 M, 50 ml). The organic layer was washed with saturated sodium hydrogen carbonate solution (50 ml), brine (20 ml), dried (MgSO₄) and the solvent was removed in vacuo. The crude product was then purified by column chromatography (SiO₂; methanol-dichloromethane 1-19) to afford the title compound as a pale yellow oil (0.73 g, 98%); m/z 572.02 (MH⁺).

### Intermediate 110: 2-(5-tert-butyl-2-methylphenoxy)-N-(4,6-dimethoxy-2-((1-((2-(trimethylsilyl)ethoxy)methyl)-1H-imidazol-2-yl)methylamino)pyrimidin-5-yl)thiazole4-carboxamide

The title compound was prepared from 2-bromo-N-(4,6-dimethoxy-2-((1-((2-(trimethylsilyl)ethoxy)methyl)-1H-imidazol-2-yl)methylamino)pyrimidin-5-yl)thiazole-4-carboxamide (Intermediate 109, 0.73 g, 1.3 mmol) and 5-tert-butyl-2-methylphenol (Intermediate 10, 0.42 g, 2.6 mmol) according to the procedure outlined for Intermediate 1. It was isolated as a pale yellow oil (0.45 g, 54%). ¹H NMR δ 7.9 (1H, s), 7.6 (1H, s), 7.2-7.3 (3H, m), 6.9-7 (2 H, m), 5.6 (1H, br t), 5.3 (2H, s), 4.73-4.8 (2H, m), 3.88 (6H, s), 3.5 (2H, t), 2.26 (3H, s), 1.31 (9H, s) 0.91 (2H, t) and 0 (9H, s); m/z 654.21 (MH⁺).

### Intermediate 111: tert-Butyl 2-(4,6-dimethoxy-5-nitropyrimidin-2-Ylamino)ethylcarbamate

tert-Butyl 2-aminoethylcarbamate (3.65 g, 22.8 mmol) was added to a mixture of 2-chloro-4,6-dimethoxy-5-nitropyrimidine (Intermediate 13, 5.0 g, 22.8 mmol) and triethylamine (3.2 ml, 22.8 mmol) in ethanol (50 ml). The solution was stirred at ambient temperature (2 h), the solvent was removed in vacuo and the crude product was then purified by column chromatography (silica; ethyl acetate-petroleum ether 1:1) to afford the title compound as a yellow oil (7.42 g, 79%); m/z 344.11 (MH⁺).

### Intermediate 112: N1-(4,6-dimethoxy-5-nitropyrimidin-2-yl)ethane-1,2-diamine

A mixture of tert-butyl 2-(4,6-dimethoxy-5-nitropyrimidin-2-ylamino)ethyl carbamate (Intermediate 111, 7.4 g, 21.5 mmol) in trifluoroacetic acid:dichloromethane 1:1 (40 ml) was made up and stirred at ambient temperature (0.5 h). The material was partitioned between dichloromethane and saturated aqueous sodium hydrogen carbonate solution before the organic layer was separated and dried to give the title compound as a bright yellow solid (3.1 g. 60%). ¹H NMR (d⁶-DMSO) δ 8.13 (1H, br s), 3.94 (3H, s), 3.90 (3H, s) 3.3-3.33 (2H, m), 2.7-2.72 (2H, m) and 1.82 (2H, br s).

### Intermediate 113: 1-(4,6-dimethoxy-5-nitropyrimidin-2-yl)imidazolidin-2-one

1,1′-Carbonyldiimidazole was added to a suspension of N1-(4,6-dimethoxy-5-nitropyrimidin-2-yl)ethane-1,2-diamine (Intermediate 112, 3.1 g, 13 mmol) in anhydrous THF (60 ml) and the mixture was heated to 60° C. for 2 hours. The resulting imidazolide (yellow solid) was filtered off, washed with THF and then suspended in a mixture of 1,4-dioxan:N-methylpyrrolidinone 6:1 (35 ml). The mixture was heated (120° C., 3 h), cooled to ambient temperature and the resulting white solid was filtered off to afford the title compound (1.7 g, 49%). ¹H NMR δ 4.97 (1H, br s), 4.17 (2H, t), 4.09 (6H, s), 3.58 (2H, t); m/z 270.1 (MH⁺)

### Intermediate 114: 1-(5-Amino-4,6-dimethoxypyrimidin-2-yl)imidazolidin-2-one

The title compound was prepared from 1-(4,6-dimethoxy-5-nitropyrimidin-2-yl)imidazolidin-2-one (Intermediate 113, 1.0 g, 3.7 mmol) by the same procedure as outlined for 2-(2-tert-butoxyethoxy)-4,6-dimethoxypyrimidin-5-amine (Intermediate 35). It was isolated as a pale brown solid (860 mg, 97%). m/z 240.08 (MH⁺).

### Intermediate 115: 2-bromo-N-(4,6-dimethoxy-2-(2-oxoimidazolidin-1-yl)pyrimidin-5-yl)thiazole-4-carboxamide

To a mixture of 1-(5-amino-4,6-dimethoxypyrimidin-2-yl)imidazolidin-2-one (Intermediate 114, 0.86 g, 3.6 mmol), 2-bromothiazole-4-carboxylic acid (Intermediate 41, 0.75 g, 3.6 mmol), triethylamine (1.0 ml, 7.16 mmol) and dichloromethane (40 ml) was added HATU (1.4 g, 3.8 mmol). The reaction was stirred at ambient temperature for 18 h. The solvent was removed in vacuo and then the mixture divided between ethyl acetate (50 ml) and dilute hydrochloric acid (0.1 M, 50 ml). The organic layer was washed with saturated aqueous sodium hydrogen carbonate solution (50 ml), brine (20 ml), dried (MgSO₄) and the solvent was removed in vacuo. The crude product was then purified by column chromatography (silica; methanol-dichloromethane 1-19) to afford the title compound as a pale yellow oil (1.4 g, 89%); m/z 430.94 (MH⁺).

### Intermediate 116: 4-tert-Butyl-2-hydroxybenzonitrile

To a solution of tert-butylphenol (5 g, 33.3 mmol) in anhydrous 1,2-dichloroethane (130 ml) at −10° C. was added boron trichloride (36.6 mmol, 36.6 ml of a 1 M solution in dichloromethane) under nitrogen and the resulting mixture stirred for 10 minutes. After removal of the cooling bath, the mixture was stirred at room temperature for 50 minutes. Trichloroacetonitrile (Cl₃CCN; 5.77 g, 40.0 mmol, 4 ml) was added and the reaction was heated under reflux for 17 h (F. Bigi, R. Maggi, G. Sartori, and G. Casnati, Gazz. Chim. Ital. 1992, 122, 283.). After cooling, the reaction mixture was quenched by addition of a suspension of potassium carbonate (25 g, 181.2 mmol) in methanol (80 ml) and the mixture was refluxed for 1.5 hours. The reaction was then allowed to cool to room temperature, the solids were removed by filtration and the solvent evaporated. Water was added to the residue and the mixture was extracted with diethylether. The organic layer was washed with dilute hydrochloric acid and water, then dried (MgSO₄) and evaporate the solvent to give the title compound (2.46 g, 14.1 mmol, 42%) after purification by column chromatography (silcac, 10% ethylacetate in petrol).

### Intermediate 117: tert-butyl 2-(5-(2-(5-tert-butyl-2-cyanophenoxy)thiazole-4-carboxamido)-4,6-dimethoxypyrimidin-2-ylamino)ethyl(isopropyl)carbamate

4-t-butyl-2-hydroxybenzonitrile (Intermediate 116; 648 mg, 3.70 mmol) was dissolved in anhydrous THF (15 ml) and potassium tert-butoxide (297 mg, 2.64 mmol) was added. The reaction mixture was stirred for 1.15 hours at 80° C. before tert-butyl 2-(5-(2-bromothiazole-4-carboxamido)-4,6-dimethoxypyrimidin-2-ylamino)ethyl(isopropyl)carbamate (Intermediate 45; 1.44 g, 2.64 mmol) was added in DMSO. The reaction was stirred at 80° C. under nitrogen for 4 days and upon cooling to room temperature, quenched with hydrochloric acid (2 N) The resulting mixture was extracted with into ethyl acetate, washed with water and brine and dried (MgSO₄). Evaporation of the solvent afforded the title compound (1.18 g, 1.85 mmol, 70%) after purification by column chromatography (silica gel 50% ethyl acetate in petrol).

### Intermediate 118: 2-(4,6-dimethoxy-5-nitropyrimidin-2-ylamino)-ethanesulfonamide

To a solution of 2-aminoethane sulphonamide hydrochloride (200 mg, 1.25 mmol) absolute ethanol (5 ml) was added triethylamine (126.5 mg, 0.18 ml). 2-chloro-4,6-dimethoxy-5-nitropyrimidine (Intermediate 13; 219 mg, 1.00 mmol) was added slowly at 0° C. and the reaction was stirred overnight at room temperature. The solvent was evaporated and the residue was partioned between ethyl acetate and saturated aqueous sodium bicarbonate solution. The mixture was extracted with ethyl acetate and the organic layer washed with brine and dried (MgSO₄). Evaporation of the solvent afforded the desired product (152 mg, 0.50 mmol) after trituration from an ethyl acetate/petrol mixture.

### Intermediate 119: 2-(5-amino-4,6-dimethoxypyrimidin-2-ylamino)-ethanesulfonamide

### 2-(4,6-dimethoxy-5-nitropyrimidin-2-ylamino)-ethanesulfonamide

(Intermediate 118; 152 mg, 0.50 mmol) was dissolved in ethanol (5 ml) and purged with nitrogen. Ammonium formate (315 mg, 5.0 mmol) was added followed by palladium hydroxide (15 mg, 10% weight). The reaction was stirred at 50° C. under nitrogen for 3 hours before being allowed to warm to room temperature and the resulting suspension was filtered and the solvent was evaporated. The residue was taken up into ethyl acetate and washed sequentially with saturated aqueous sodium bicarbonate solution and brine before being dried (MgSO₄). Evaporation of the solvent gave the required amine (86 mg, 62%).

### Intermediate 120: 2-(1,3-dioxoisoindolin-2-yl)-N-isopropyl-ethanesulfonamide

2-Phthalamidoethane sulfonyl chloride (200 mg, 0.73 mmol) was dissolved in isopropylamine (1 ml, 11.74 mmol) and the reaction was stirred under nitrogen until complete (LC-MS). The solvent was evaporated and the material was divided between ethyl acetate and saturated aqueous sodium bicarbonate solution, the organic layer was separated before the inorganic portion was extracted further with ethyl acetate and the combined organinc extracts were washed with brine and dried (MgSO₄). Evaporation of the solvent afforded the title compound (230 mg, 99%) use as a crude in the next step.

### Intermediate 121: 2-Amino-N-isopropylethanesulfonamide

2-(1,3-Dioxoisoindolin-2-yl)-N-isopropyl-ethanesulfonamide (Intermediate 120; 230 mg, 0.78 mmol) was dissolved in ethanol (5 ml) and hydrazine hydrate (0.1 ml, 1.55 mmol) was added. The reaction mixture was stirred at 60° C. overnight and upon cooling to room temperature was filtered through celite (flushing with ethanol). In order to obtain homogeneous material, the crude product was filtered through an SPE cartridge of MPTsOH flushing first with methanol and then with methanol/dichloromethane to remove the impurities followed by 2 N NH₃/methanol to retrieve the pure title compound (129 mg, 99%).

### Intermediate 122: 2-(5-nitro-4,6-dimethoxypyrimidin-2-ylamino)-N-isopropylethanesulfonamide

2-Amino-N-isopropylethanesulfonamide (Intermediate 121; 130 mg, 0.78 mmol) was dissolved in ethanol (3 ml) and the 2-chloro-4,6-dimethoxy-5-nitropyrimidine (Intermediate 13; 172 mg, 0.78 mmol) was added slowly at 0° C. The reaction was stirred overnight at room temperature. The solvent was evaporated and the residue was divided between ethyl acetate and saturated aqueous sodium bicarbonate solution. The inorganic portion was extracted with ethyl acetate and the organic layer washed with brine and dried (MgSO₄). Evaporation of the solvent afforded the desired product (66 mg of pure compound, 0.19 mmol, 24% of pure material) after purification using an NH propyl cartridge (Biotage AB; 2% methanol in dichloromethane).

### Intermediate 123: 2-(5-amino-4,6-dimethoxypyrimidin-2-ylamino)-N-isopropylethanesulfonamide

2-(5-nitro-4,6-dimethoxypyrimidin-2-ylamino)-N-isopropylethanesulfonamide (Intermediate 122; 66 mg, 0.19 mmol) was dissolved in ethanol (2 ml) and purged with nitrogen. Ammonium formate (126 mg, 2.0 mmol) was added followed by palladium hydroxide (7 mg, 10% weight). The reaction was stirred at 50° C. under nitrogen for 3 hours until complete (LC-MS) and then allowed to cool at room temperature, filtered and the solvent was evaporated. The residue was dissolved in ethyl acetate and washed with saturated aqueous sodium bicarbonate solution and brine and dried (MgSO₄). Evaporation of the solvent gave the title compound (52 mg, 86%).

### Intermediate 124: 3-(4,6-dimethoxy 5-nitropyrimidin-2-ylamino)propanenitrile

3-aminopropionitrile (400 mg, 2.85 mmol) was dissolved in ethanol (5 ml) and 2-chloro-4,6-dimethoxy-5-nitropyrimidine (Intermediate 13; 417 mg, 1.90 mmol) was added slowly at 0° C. The reaction was stirred overnight at room temperature. The solvent was evaporated and the residue was divided between ethyl acetate and saturated aqueous sodium bicarbonate solution. The mixture was extracted with ethyl acetate and the organic layer washed with brine and dried (MgSO₄). Evaporation of the solvent afforded the desired product (410 mg, 1.62 mmol, 85%) after purification using an NH propyl cartridge (Biotage AB; 2% methanol in dichloromethane).

### Intermediate 125: 3-(5-amino-4,6-dimethoxypyrimidin-2-ylamino)propanenitrile

3-(4,6-dimethoxy-5-nitropyrimidin-2-ylamino)propanenitrile (Intermediate 124; 410 mg, 0.78 mmol) was dissolved in ethanol (8 ml) and purged with nitrogen. Ammonium formate (490 mg, 7.8 mmol) was added followed by palladium hydroxide (41 mg, 10% weight). The reaction was stirred at 50° C. under nitrogen for 3 hours and upon cooling to room temperature, was filtered and concentrated to dryness. The residue was dissolved in ethyl acetate and washed with saturated aqueous sodium bicarbonate solution and brine and dried over (MgSO₄). Evaporation of the solvent gave the required amine (175 mg, 99%).

### Intermediate 126: ethyl 3-(4,6-dimethoxy-5-nitropyrimidin-2-ylamino)propanoate

β-alanine ethyl ester hydrochloride (200 mg, 1.30 mmol) was dissolved in ethanol (5 ml) and 2-chlorb-4,6-dimethoxy-5-nitropyrimidine (Intermediate 13; 220 mg, 1.00 mmol) was added slowly at 0° C. The reaction was stirred overnight at room temperature before the solvent was evaporated and the residue was divided between ethyl acetate and saturated aqueous sodium bicarbonate solution. The inorganic portion was extracted with ethyl acetate and the combined organic layer was washed with brine and dried (MgSO₄). Evaporation of the solvent afforded the desired product (154 mg, 0.51 mmol, 51%) after trituration from a mixture of petrol and ethyl acetate.

### Intermediate 127: ethyl 3-(5-amino-4,6-dimethoxypyrimidin-2-ylamino)propanoate

Ethyl 3-(4,6-dimethoxy-5-nitropyrimidin-2-ylamino)propanoate (intermediate 126: 154 mg, 0.51 mmol) was dissolved in ethanol (5 ml) and purged with nitrogen. Ammonium formate (323 mg, 5.1 mmol) was added followed by palladium hydroxide (15 mg, 10% weight). The reaction was stirred at 50° C. under nitrogen for 3 hours before it was allowed to cool to room temperature, filtered and the solvent was evaporated. The residue was dissolved in ethyl acetate and washed with saturated sodium bicarbonate solution and brine and dried (MgSO₄). Evaporation of the solvent gave the required amine (136 mg, 99%).

### Intermediate 128: ethyl 3-(5-(2-(5-tert-butyl-2-methylphenoxy)thiazole-4-carboxamido)-4,6-dimethoxypyrimidin-2-ylamino)propanoate

2-(5-tert-Butyl-2-methylphenoxy)thiazole-4-carboxylic acid (Intermediate 18; 147 mg, 0.50 mmol) and ethyl 3-(5-amino-4,6-dimethoxypyrimidin-2-ylamino)propanoate (Intermediate 127; 136 mg, 0.50 mmol) were dissolved in dichloromethane (4 ml) and triethylamine (0.14 ml, 1.0 mmol) and HBTU (284 mg, 0.75 mmol) were added at room temperature. The reaction was stirred until complete (LC-MS) and then saturated aqueous sodium bicarbonate solution was added and the solvent was evaporated. The residue was extracted into ethyl acetate, washed with water and brine, and dried (MgSO₄). Evaporation of the solvent followed by purification by column chromatography (silica gel; 2% methanol and 2% 0.880 aqueous ammonia solution in dichloromethane) afforded the title compound (220 mg, 81%).

### Example 1

### 2-(3-tert-butylphenoxy)-N-(4,6-dimethoxy-2-(3-morpholinopropylamino)pyrimidin-5-yl)oxazole-4-carboxamide

To a cooled (−30° C.) solution of 4,6-dimethoxy-N-(3-morpholinopropyl)pyrimidine-2,5-diamine (prepared according to the procedure in WO-A-02/098363; 1.09 g, 3.67 mmol) was added dropwise a solution of trimethylaluminium (2 M solution in toluene, 5.5 ml, 11 mmol) and the resulting mixture was allowed to warm to −20° C. over 30 min and then to room temperature over 45 min. This mixture was then added dropwise to a cooled (0° C.) solution of ethyl 2-(3-tert-butylphenoxy)oxazole-4-carboxylate (Intermediate 1, 530 mg, 1.83 mmol) in dichloromethane. The resulting mixture was allowed to warm to room temperature over 30 min and then heated to 40° C. for 16 h. Upon cooling the reaction was then quenched with saturated aqueous ammonium acetate (30 ml, CARE EXOTHERM). The aqueous phase was separated and then extracted with ethyl acetate (4×25 ml) and the combined organic extracts were washed with brine (25 ml) and dried (MgSO₄) before being concentrated in vacuo. The crude product was purified by column chromatography (SiO₂; 1% acetic acid in dichloromethane to 1% acetic acid and 5% methanol in dichloromethane). The pure fractions were concentrate, diluted with ethyl acetate and washed with saturated aqueous sodium hydrogen carbonate solution. The aqueous layer was then extracted into ethyl acetate, dried (MgSO₄) and concentrated in vacuo to afford the title compound (314 mg, 32%). M.p. 85° C., R_{f} 0.19 (19:1 dichloromethane-methanol). ¹H NMR δ 7.83 (1H, s), 7.45 (1H, s), 7.21-7.32 (3H, m), 7.13-7.18 (1H, m), 5.68 (1H, br, m), 3.79 (6H, s), 3.66-3.69 (4H, m), 3.37-3.42 (2H, m), 2.40-2.48 (6H, m), 1.67-1.75 (2H, m) and 1.26 (9H, s); m/z 541.3 (MH⁺)

### Example 2

### 2-(3-tert-butylphenoxy)-N-(4,6-dimethoxy-2-(3-morpholinopropylamino)pyrimidin-5-yl)thiazole-4-carboxamide

Prepared according to the method directly above for Example 1 from ethyl 2-(3-tert-butylphenoxy)thiazole-4-carboxylate (Intermediate 2) and 4,6-dimethoxy-N-(3-morpholinopropyl)pyrimidine-2,5-diamine (prepared according to the procedure in WO-A-02/098363) with the exception that the final reaction mixture was allowed to stir at room temperature for 60 h. Work-up and purification as described afforded the title compound (50%). M.p. 68° C., R_{f} 0.32 (19:1 dichloromethane-methanol). ¹H NMR δ 7.84 (1H, s), 7.60 (1H, s), 7.20-7.32 (3H, m), 7.05-7.8 (1H, m), 5.68 (1H, br, m), 3.79 (6H, s), 3.66-3.69 (4H, m), 3.37-3.42 (2H, m), 2.38-2.45 (6H, m), 1.65-1.75 (2H, m) and 1.26 (9H, s); m/z 557.2 (MH⁺).

### Example 3

### 2-(3,3-dimethyl-2,3-dihydro-1H-inden-5-yloxy)-N-(2-(2-(dimethylamino)ethylamino)-4,6-dimethoxypyrimidin-5-yl)oxazole-4-carboxamide

The title compound was prepared according to the method outlined in Example 1 from N-(2-(dimethylamino)ethyl)-4,6-dimethoxypyrimidine-2,5-diamine (Intermediate 4) and ethyl 2-(3,3-dimethyl-2,3-dihydro-1H-inden-5-yloxy)oxazole-4-carboxylate (Intermediate 6) with the exception that after aqueous work up the crude compound was purified first by cation exchange (Argonaut MP-TsOH, elution 2 N ammonia in methanol) and then by column chromatography (SiO₂, elution 19:1-dichloromethane-methanol and 0.5% ammonia). The title compound was isolated as a yellow glass (11% yield). R_{f}=0.10 (1:9-methanol-dichloromethane with 0.1% 0.88 aqueous ammonia solution). ¹H NMR δ 7.82 (1H, s), 7.44 (1H, broad s), 7.14-7.16 (1H, m), 7.04-7.07 (1H, m), 6.98-6.99 (1H, m), 5.30 (1H, br, t), 3.79 (6H, s), 3.38 (2H, q), 2.83 (2H, t), 2.42 (2H, t), 2.20 (6H, s), 1.91 (2H, t) and 1.20 (6H, s); m/z 497.1 (MH⁺).

### Example 4

### 2-(3,3-dimethyl-2,3-dihydro-1H-inden-5-yloxy)-N-(2-(2-(dimethylamino)ethylamino)-4,6-dimethoxypyrimidine-5-yl)thiazole-4-carboxamide

The title compound was prepared according to the method outlined in Example 1 from N-(2-(dimethylamino)ethyl)-4,6-dimethoxypyrimidine-2,5-diamine (Intermediate 4) and ethyl 2-(5-tert-butyl-2-methylphenoxy)thiazole-4-carboxylate (Intermediate 7) with the exception that after aqueous work up the crude compound was purified first by cation exchange (Argonaut MP-TsOH, elution 2 N ammonia in methanol) and then by column chromatography (SiO₂, elution 5% methanol and 0.5% ammonia in dichloromethane). The title compound was isolated as a pale orange foam (49% yield). R_{f}=0.10 (1:9-methanol-dichloromethane with 0.1% 0.88 aqueous ammonia solution). ¹H NMR δ 7.94 (1H, br, s), 7.67 (1H, s), 7.23-7.25 (1H, m), 7.06-7.08 (2H, m), 5.40 (1H, br, t), 3.89 (6H, s), 3.48 (2H, q), 2.93 (2H, t), 2.52 (2H, t), 2.29 (6H, s), 2.0 (2H, t) and 1.28 (6H, s); m/z 513.2 (MH⁺).

### Example 5

### 2-(5-tert-Butyl-2-methylhenoxy)-N-(2-(2-(dimethylamino)ethylamino)-4,6-dimethoxypyrimidin-5-yl)thiazole-4-carboxamide

The title compound was prepared according to the method outlined in Example 1 from N-(2-(dimethylamino)ethyl)-4,6-dimethoxypyrimidine-2,5-diamine (Intermediate 4) and ethyl 2-(5-tert-butyl-2-methylphenoxy)thiazole-4-carboxylate (Intermediate 12) with the exception that after aqueous work up the crude compound was purified first by column chromatography (C18, 0.05% ammonia in water and acetonitrile, gradient elution 5 to 95% acetonitrile) and then by cation exchange (Argonaut MP-TsOH, elution 2 N ammonia in methanol). The title compound was isolated as a pale brown glass (5% yield). R_{f}=0.10 (1:9-methanol-dichloromethane with 0.1% 0.88 aqueous ammonia solution). ¹H NMR δ7.87 (1H, br, s), 7.56 (1H, s), 7.17-7.19 (3H, m), 5.30 (1H, br, t), 3.80 (6H, s), 3.48 (2H, q), 2.43 (2H, t), 2.20 (6H, s) and 1.24 (6H, s); m/z 515.2 (MH⁺).

### Example 6

### 2-(3-tert-Butylphenoxy)-N-(2-(2-(dimethylamino)ethylamino)-4,6-dimethoxypyrimidin-5-yl)oxazole-4-carboxamide

The title compound was prepared according to the method outlined in Example 1 from N-(2-(dimethylamino)ethyl)-4,6-dimethoxypyrimidine-2,5-diamine (Intermediate 4) and ethyl 2-(3-tert-butylphenoxy)oxazole-4-carboxylate (Intermediate 1) with the exception that the crude compound after aqueous work up was purified first by cation exchange (Argonaut MP-TsOH, elution 2 N ammonia in methanol) and then by column chromatography (SiO2, elution 5% methanol and 0.5% ammonia in dichloromethane). The title compound was isolated as a yellow glass (26% yield). R_{f}=0.10 (1:9-methanol-dichloromethane with 0.1% 0.88 aqueous ammonia solution). ¹H NMR δ 7.84 (1H, s), 7.46 (1H, br, s), 7.23-7.32 (3H, m), 7.13-7.16 (1H, m), 5.30 (1H, br, t), 3.79 (6H, s), 3.38 (2H, q), 2.42 (2H, t), 2.20 (6H, s), and 1.27 (9H, s); m/z 485.2 (MH⁺).

### Example 7

### 2-(3-tert-Butylphenoxyy-N-(2-(2-(dimethylamino)ethylamino)-4,6-dimethoxypyrimidin-5-yl)thiazole-4-carboxamide

The title compound was prepared according to the method outlined in Example 1 from N-(2-(dimethylamino)ethyl)-4,6-dimethoxypyrimidine-2,5-diamine (Intermediate 4) and ethyl 2-(3-tert-butylphenoxy)thiazole-4-carboxylate (Intermediate 2) with the exception that after aqueous work up the crude compound was purified first by column chromatography (C18, 0.05% ammonia in water and acetonitrile, gradient elution 5 to 95% acetbnitrile) and then by cation exchange (Argonaut MP-TsOH, elution 2 N ammonia in methanol). The title compound was isolated as a grey glass (38%). R_{f}=0.10 (1:9-methanol-dichloromethane with 0.1% 0.88 aqueous ammonia solution).

¹H NMR δ 7.94 (1H, br, s), 7.70 (1H, s), 7.33-7.40 (3H, m), 7.12-7.15 (1H, m), 5.42 (1H, br, t), 3.89 (6H, s), 3.48 (2H, q), 2.52 (2H, t), 2.29 (6H, s) and 1.35 (9H, s); m/z 501.2 (MH⁺).

### Example 8

### 2-(5-tert-Butyl-2-methylphenoxy)-N-4,6-dimethoxy-2-(2-(methylamino)ethylamino)pyrimidin-5-yl)oxazole-4-carboxamide

To a solution of tert-butyl 2-(5-(2-(5-tert-butyl-2-methylphenoxy)oxazole-4-carboxamido)-4,6-dimethoxypyrimidin-2-ylamino)ethyl(methyl)carbamate (Intermediate 17; 0.503 g, 0.86 mmol) in DCM (10 ml) was added trifluoroacetic acid (10 ml). The solution was stirred for a further hour at ambient temperature and then the volatiles were removed in vacuo. The title compound was isolated by cation exchange (Argonaut MP-TsOH, elution 2 N ammonia in methanol) followed by column chromatography (SiO₂, elution 5% methanol in dichloromethane) as an off-white foam (0.1 g, 24%). ¹H NMR δ 7.85 (1H, s), 7.13-7.21 (3H, m), 5.61 (1H, br, t), 3.78 (6 H, br, s), 3.56 (2H, q), 3.41 (1H, br, s), 2.91 (2H, t), 2.49 (3H, s), 2.18 (3H, s) and 1.25 (9H, s). m/z 485.25 (MH⁺).

### Example 9

### 2-(5-tert-Butyl-2-methylphenoxy)-N-(4,6-dimethoxy-2-(2-(methylamino)ethylamino)pyrimidin-5-yl)thiazole-4-carboxamide

The title compound was prepared according to the method outlined in Example 8 from tert-butyl 2-(5-(2-(5-tert-butyl-2-methylphenoxy)thiazole-4-carboxamido)-4,6-dimethoxypyrimidin-2-ylamino)ethyl(methyl)carbamate (Intermediate 19; 0.54 g, 0.9 mmol) and isolated as a pale green glass (0.31 g, 68%). ¹H NMR δ 7.88 (1H, br, s), 7.56 (1H, s), 7.15-7.20 (3H, m), 5.32 (1H, br, t), 3.8 (6H, br, s), 3.49 (2H, q), 2.81 (2H, t), 2.53 (1H, br, s), 2.43 (3H, s), 2.19 (3H, s) and 1.24 (9H, s); m/z 485.25 (MH⁺).

### Example 10

### N-(4,6-Dimethoxy-2-(2-(methylamino)ethylamino)pyrimidin-5-yl)-2-(3,3,6-trimethyl-2,3-dihydro-1H-inden-5-yloxy)oxazole-4-carboxamide

The title compound was prepared according to the method outlined in Example 8 from tert-butyl 2-(4,6-dimethoxy-5-(2-(3,3,6-trimethyl-2,3-dihydro-1H-inden-5-yloxy)oxazole-4-carboxamido)pyrimidin-2-ylamino)ethyl(methyl)carbamate (Intermediate 22; 0.53 g, 0.9 mmol) and isolated as an off-white solid (0.2 g, 45%). ¹H NMR δ 7.82 (1H, s), 7.45 (1H, br, s), 7.02 (1H, s), 6.93 (1H, s), 5.41 (1H, t), 3.78 (6H, s), 3.51 (2H, q), 2.79-2.85 (4H, m), 2.45 (3H, s), 2.16 (3H, s), 1.89 (2H, t) and 1.18 (6H, s); m/z 497.22 (MH⁺).

### Example 11

### N-(4,6-Dimethoxy-2-(2-(methylamino)ethylamino)pyrimidin-5-yl)-2-(3,3,6-trimethyl-2,3-dihydro-1H-inden-5-yloxy)thiazole-4-carboxamide

The title compound was prepared according to the method outlined in Example 8 from tert-butyl 2-(4,6-dimethoxy-5-(2-(3,3,6-trimethyl-2,3-dihydro-1H-inden-5-yloxy)thiazole-4-carboxamido)pyrimidin-2-ylamino)ethyl(methyl)carbamate (Intermediate 25; 0.44 g, 0.7 mmol) and isolated as an off-white glass (0.2 g, 55%). ¹H NMR δ 7.89 (1H, br, s), 7.55 (1H, s), 7.04 (1H, s), 6.91 (1H, s), 5.24 (1H, t), 3.8 (6H, s), 3.46 (2H, q), 2.81 (2H, t), 2.76 (2H, t), 2.41 (3H, s), 2.17 (3H, s), 1.89 (2H, t), 1.86 (1H, br, s) and 1.17 (6H, s); m/z 512.62 (MH⁺).

### Example 12

### N-(4,6-Dimethoxy-2-(2-(methylamino)ethylamino)pyrimidin-5-yl)-2-(5-isopropyl-2-methylphenoxy)thiazole-4-carboxamide

The title compound was prepared according to the method outlined in Example 8 from tert-butyl 2-(5-(2-(5-isopropyl-2-methylphenoxy)thiazole-4-carboxamido)-4,6-dimethoxypyrimidin-2-ylamino)ethyl(methyl)carbamate (Intermediate 28; 0.7 g, 1.2 mmol) and isolated as an off-white foam (0.31 g, 51%). ¹H NMR δ 7.97 (1H, br, s), 7.66 (1H, s), 7.24 (1H, d), 7.10-7.13 (2H, m), 5.35 (1H, t), 3.89 (6H, s), 3.56 (2H, q), 2.93 (2H, m), 2.87 (2H, t), 2.5 (3H, s), 2.28 (3H, s), 2.1 (1H, br, s), 1.27 (3H, S) and 1.25 (3H, s); m/z 487.24 (MH⁺).

### Example 13

### 2-(5-tert-Butyl-2-methylphenoxy)-N-(2-(2-hydroxyethylamino)-4,6-dimethoxypyrimidin-5-yl)thiazole-4-carboxamide

To a solution of 2-(5-tert-butyl-2-methylphenoxy)-N-(2-(2-(tert-butyldimethylsilyloxy)ethylamino)-4,6-dimethoxypyrimidin-5-yl)thiazole-4-carboxamide (Intermediate 32; 0.7 g, 1.17 mmol) in 1:1 tetrahydrofuran:water (6 ml) was added glacial acetic acid (9 ml). The solution was stirred at ambient temperature for a further 18 h at which time TLC showed that the reaction had gone to completion. The reaction mixture was partitioned between dichloromethane (25 ml) and 2 N NaOH (25 ml) and after rigorous shaking the aqueous later was separated and extracted further with dichloromethane (1×25 ml). The combined organic phase was washed with brine (50 ml) and dried (MgSO₄) before being concentrated in vacuo. The mixture was purified by column chromatography (SiO₂; elution 2% methanol in dichloromethane). The title compound was isolated as an off-white foam (0.6 g, 84%). ¹H NMR δ 7.86 (1H, br, s), 7.56 (1H, s), 7.16-7.19 (3H, m), 5.23 (1H, t), 3.8 (6H, s), 3.75 (2H, q), 3.51 (2H, m), 3.01 (1H, t), 2.19 (3H, s), 1.23 (9H, s); m/z 487.57 (MH⁺).

### Example 14

### 2-(5-tert-Butyl-2-methylphenoxy)-N-(2-(2-(isopropylamino)ethylamino)-4,6-dimethoxypyrimidin-5-yl)thiazole-4-carboxamide

In a sealed tube isopropylamine (0.264 ml, 3.1 mmol) was added to a solution of 2-(5-(2-(5-tert-butyl-2-methylphenoxy)thiazole-4-carboxamido)-4,6-dimethoxypyrimidin-2-ylamino)ethyl methanesulfonate (Intermediate 33; 0.175 g, 0.31 mmol) in tetrahydrofuran (10 ml). The tube was sealed and heated at 65° C. for 6 h after which time the solvent was removed in vacuo. The crude material was purified by cation exchange (Argonaut MP-TsOH, elution 2 N ammonia in methanol) followed by column chromatography (SiO₂, elution 10% methanol in dichloromethane with 1% NH₄OH added). The title compound was isolated as a pale brown oil (0.03 g, 18%). ¹H NMR δ 7.9 (1H, br, s), 7.56 (1H, s), 7.15-7.2 (3H, m), 6.21 (1H, t), 3.8 (6H, s), 3.76-3.82 (2H, m), 3.24 (1H, t), 3.13 (2H, t), 2.19 (3H, s), 1.36 (3H, s), 1.35 (3H, s), 1.24 (9H, s); m/z 529.28 (MH⁺).

### Example 15

### N-(2-(2-tert-Butoxyethoxy)-4,6-dimethoxypyrimidin-5-yl)-2-(5-tert-butyl-2-methylphenoxy)thiazole-4-carboxamide

To a solution of ethyl 2-(5-tert-butyl-2-methylphenoxy)thiazole-4-carboxylate (Intermediate 12; 0.562 g, 1.93 mmol), 2-(2-tert-butoxyethoxy)-4,6-dimethoxypyrimidin-5-amine (Intermediate 35; 0.5 g, 1.93 mmol) and triethylamine (0.542 ml, 3.86 mmol) in dichloromethane (20 ml) was added 0 benzotriazol-1-N,N,N′,N′-tetramethylammonium hexafluorophosphate (HBTU, 0.805 g, 2.12 mmol). The solution was stirred for a further 18 hours at ambient temperature whereby the reaction was seen to be complete by TLC analysis. The material was washed with brine and dried (MgSO₄). The solvent was removed in vacuo. The title compound was isolated by chromatography (SiO₂, elution 1:7 ethyl acetate-petroleum ether) as a clear glass (0.663 g, 63%). ¹H NMR δ 8.05 (1H, s), 7.69 (1H, s), 7.28-7.31 (3H, m), 4.48 (2H, t), 4.0 (6H, s), 3.77 (2H, t), 2.3 (3H, s), 1.35 (9H, s), and 1.26 (9H, s); m/z 545.27 (MH⁺)

### Example 16

### 2-(5-tert-Butyl-2-methylphenoxy)-N-(2-(2-hydroxyethoxy)-4,6-dimethoxypyrimidin-5-yl)thiazole-4-carboxamide

Trifluoroacefic acid (10 ml) was added to a solution of N-(2-(2-tert-butoxyethoxy)-4,6-dimethoxypyrimidin-5-yl)-2-(5-tert-butyl-2-methylphenoxy)thiazole-4-carboxamide (Example 15; 0.630 g, 1.16 mmol) in dichloromethane (10 ml). The solution was stirred for 120 minutes at ambient temperature and then the volatiles were removed in vacuo. The material was dissolved in ethyl acetate (20 ml) and washed with aqueous sodium hydroxide (2 N, 10 ml). The organic layer was washed with brine and dried (MgSO₄). The solvent was removed in vacuo and the title compound was isolated as a white foam (0.4 g, 70%). ¹H NMR δ 8.07 (1H, s), 7.7 (1H, s), 7.28-7.3 (3H, m), 4.51-4.53 (2H, m), 4.0-4.03 (2H, m), 4.0 (6H, s), 2.38 (1H, br s), 2.30 (3H, s) and 1.35 (9H, s); m/z 489.21 (MH⁺).

### Example 17

### 2-(5-Chloro-2-methylphenoxy)-N-(2-(2-hydroxyethylamino)-4,6-dimethoxypyrimidin-5-yl)thiazole-4-carboxamide

To a solution of N-(2-(2-(tert-butyldimethylsilyloxy)ethylamino)-4,6-dimethoxypyrimidin-5-yl)-2-(5-chloro-2-methylphenoxy)thiazole-4-carboxamide (Intermediate 37; 0.57 g, 0.98 ml) in 1:1 tetrahydrofuran:water (6 ml) was added glacial acetic acid (9 ml). The solution was stirred at ambient temperature for a further 18 hours at which time TLC showed that the reaction had gone to completion. The solvent was removed in vacuo and the reaction mixture was partitioned between ethyl acetate (25 ml) and aqueous sodium hydroxide solution (2 N; 25 ml). After rigorous shaking the aqueous later was separated and extracted further with ethyl acetate (1×25 ml). The combined organic phase was washed with brine (50 ml) and dried (MgSO₄) before being concentrated in vacuo. The mixture was purified by column chromatography (SiO₂; elution 2% methanol in dichloromethane). The title compound was isolated as a white solid (0.33 g, 73%). ¹H NMR δ 7.82 (1H, br, s), 7.7 (1H, s), 7.19-7.28 (3H, m), 5.3 (1H, br t), 3.88 (6H, s), 3.80-3.84 (2H, m), 3.56-3.60 (2H, m), 3.05 (1H, br t) and 2.27 (3H, s); m/z 466.12 (MH⁺).

### Example 18

### N-(2-(2-Hydroxyethylamino)-4,6-dimethoxypyrimidin-5-yl)-2-(5-isopropyl-2-methylphenoxy)thiazole-4-carboxamide

The title compound was prepared from N-(2-(2-(tert-butyldimethyl silyloxy)ethylamino)-4,6-dimethoxypyrimidin-5-yl)-2-(5-isopropyl-2-methylphenoxy) thiazole-4-carboxamide (Intermediate 38; 0.765 g, 1.3 mmol) according to the procedure outlined for Example 17. The product was isolated as a white foam (0.4 g, 65%). ¹H NMR δ 7.95 (1H, br, s), 7.65 (1H, s), 7.2-7.23 (1H, m), 7-7.1 (2H, m), 5.43 (1H, br t), 3.87 (6H, s), 3.80 (2H, t), 3.54-3.58 (2H, m), 2.87-2.94 (1H, m) and 2.25 (3H, s), 1.25 (3H, s) and 1.23 (3H, s); m/z 474.23 (MH⁺).

### Example 19

### 2-(2,5-Dimethylphenoxy)-N-(2-(2-hydroxyethylamino)-4,6-dimethoxypyrimidin-5-yl)thiazole-4-carboxamide

The title compound was prepared from N-(2-(2-(tert-butyldimethylsilyloxy)ethylamino)-4,6-dimethoxypyrimidin-5-yl)-2-(2,5-dimethylphenoxy)thiazole-4-carboxamide (Intermediate 39; 0.54 g, 0.96 mmol) according to the procedure outlined for Example 17. The product was isolated as a clear oil (0.26 g, 61%). ¹H NMR δ 7.94 (1H, br, s), 7.65 (1H, s), 7.17-7.19 (1H, m), 7-7.05 (2H, m), 5.47 (1H, br t), 3.86 (6H, s), 3.76-3.8 (2H, m), 3.52-3.56 (2H, m), 3.39 (1H, m), 2.34 (3H, s) and 2.24 (3H, s); m/z 446.20 (MH⁺).

### Example 20

### N-(2-(2-Hydroxyethylamino)-4,6-dimethoxypyrimidin-5-yl)-2-(o-tolyloxy)thiazole-4-carboxamide

The title compound was prepared from N-(2-(2-(tert-butyldimethylsilyloxy)ethylamino)-4,6-dimethoxypyrimidin-5-yl)-2-(o-tolyloxy)thiazole-4-carboxamide (Intermediate 40; 0.42 g, 0.76 mmol) according to the procedure outlined for Example 17. The product was isolated as a white foam (0.19 g, 57%). ¹H NMR δ 7.93 (1H, br, s), 7.68 (1H, s), 7.22-7.34 (4H, m), 5.5 (1H, br t), 3.88 (6H, s), 3.8 (2H, m), 3.57 (2H, m), 3.42 (1H, m) and 2.32 (3H, s); m/z 432.17 (MH⁺).

### Example 21

### 2-(2-Chloro-5-(trifluoromethyl)phenoxy)-N-(2-(2-hydroxyethylamino)-4,6-dimethoxypyrimidin-5-yl)thiazole-4-carboxamide

To a mixture of N-(2-(2-(tert-butyldimethylsilyloxy)ethylamino)-4,6-dimethoxypyrimidin-5-yl)-2-chlorothiazole-4-carboxamide (Intermediate 36, 0.58 g, 1.23 mmol) and 2-chloro-5-(trifluoromethyl)phenol (0.5 ml, 3.7 mmol) dissolved in anhydrous THF (6 ml) was added potassium carbonate (0.51 g, 3.7 mmol). The reaction was heated in a sealed tube in the microwave at 160° C. for 30 minutes and on cooling was partitioned between ethyl acetate and water. The organic layer was washed with brine, dried (MgSO₄) and the solvent was removed in vacuo. The crude product was then purified by column chromatography (C18; 5-95% acetonitrile in 0.05% ammonium hydroxide solution) to afford the title compound as an off-white solid (335 mg, 50%). ¹H NMR δ 7.75 (1H, s), 7.70-7.73 (1H, m), 7.63-7.65 (1H, m), 7.5-7.53 (1H, m) 5.43 (1H, br t), 3.85 (6H, s), 3.8 (2H, t), 3.52-3.56 (2H, m) and 3.26 (1H, br s); m/z 520.06 (MH⁺).

### Example 22

### 2-(6-Bromo-3,3-dimethyl-2,3-dihydro-1H-inden-5-yloxy)-N-(2-(2-(isopropylamino)ethylamino)-4,6-dimethoxypyrimidin-5-yl)thiazole-4-carboxamide

Trifluoroacetic acid (10 ml) was added to a solution of tert-butyl 2-(5-(2-(6-bromo-3,3-dimethyl-2,3-dihydro-1H-inden-5-yloxy)thiazole-4-carboxamido)-4,6-dimethoxypyrimidin-2-ylamino)ethyl(isopropyl)carbamate (Intermediate 47; 0.39 g, 0.55 mmol) in dichloromethane (10 ml). The solution was stirred for 30 min at ambient temperature and then the volatiles were removed in vacuo. The material was dissolved in ethyl acetate (20 ml) and washed with aqueous sodium hydroxide (2 N; 10 ml). The organic layer was washed with brine and dried (MgSO₄). The solvent was removed in vacuo and the title compound was isolated by column chromatography (2N; NH₃ in methanol dichloromethane, 1:9) as an off-white foam (0.21 g, 63%). ¹H NMR δ 7.9 (1H, s), 7.7 (1H, s), 7.46 (1H, s), 7.12 (1H, s), 5.2 (1H, br t), 3.87 (6H, s), 3.48-3.52 (2H, m), 2.91 (2H, t), 2.81-2.85 (3H, m), 1.99 (2H, t), 1.25 (6H, s), 1.08 (3H, s) and 1.06 (3H, s); m/z 605.18 (MH⁺).

### Example 23

### 2-(6-Chloro-3,3-dimethyl-2,3-dihydro-1H-inden-5-yloxy)-N-(2-(2-(isopropylamino)ethylamino)-4,6-dimethoxypyrimidin-5-yl)thiazole-4-carboxamide

Prepared from tert-butyl 2-(5-(2-(6-chloro-3,3-dimethyl-2,3-dihydro-1H-inden-5-yloxy)thiazole-4-carboxamido)-4,6-dimethoxypyrimidin-2-ylamino)ethyl (isopropyl)carbamate (Intermediate 49; 0.35 g, 0.53 mmol) according to the procedure described for Example 22. The title compound was isolated by column chromatography (Biotage KPNH, methanol: dichloromethane, 1:9) as an off-white foam (0.19 g, 64%). ¹H NMR δ 7.88 (1H, s), 7.67 (1H, s), 7.29 (1H, s), 7.1 (1H, s), 5.25 (1H, br t), 3.86 (6H, s), 3.46-3.5 (2H, m), 2.9 (2H, t), 2.8-2.84 (3H, m), 1.98 (2H, t), 1.25 (6H, s), 1.07 (3H, s) and 1.06 (3H, s); m/z 561.17 (MH⁺)

### Example 24

### N-(2-(2-(Isopropylamino)ethylamino)-4,6-dimethoxypyrimidin-5-yl)-2-(6-methoxy-3,3-dimethyl-2,3-dihydro-1H-inden-5-yloxy)thiazole-4-carboxamide

Prepared from tert-butyl 2-(4,6-dimethoxy-5-(2-(6-methoxy-3,3-dimethyl-2,3-dihydro-1H-inden-5-yloxy)thiazole-4-carboxamido)pyrimidin-2-ylamino)ethyl(isopropyl)carbamate (Intermediate 52; 0.54 g, 0.82 mmol) according to the procedure described for Example 22. The title compound was isolated by column chromatography (Biotage KPNH, methanol-dichloromethane, 1:9) as a pale brown solid (0.43 g, 93%). ¹H NMR δ 7.97 (1H, s), 7.59 (1H, s), 7.0 (1H, s), 6.88 (1H, s), 5.23 (1H, br t), 3.86 (6H, s), 3.8 (3H, s), 3.46-3.52 (2H, m), 2.9 (2H, t), 2.8-2.84 (3H, m), 1.96 (2H, t), 1.23 (6H, s), 1.07 (3H, s) and 1.06 (3H, s); m/z 557.24 (MH⁺)

### Example 25

### N-(2-(2-(Isopropylamino)ethylamino)-4,6-dimethoxypyrimidin-5-yl)-2-(5-methoxy-2-methylphenoxy)thiazole-4-carboxamide

Prepared from tert-butyl 2-(4,6-dimethoxy-5-(2-(5-methoxy-2-methylphenoxy)thiazole-4-carboxamido)pyrimidin-2-ylamino)ethyl(isopropyl)carbamate (Intermediate 54; 0.3 g, 0.5 mmol) according to the protocol used to prepare Example 22. The title compound was isolated by column chromatography (2N NH₃ in methanol: dichloromethane, 1:9) as a pale brown solid (0.12 g, 48%). ¹H NMR δ 7.95 (1H, br s), 7.65 (1H, s), 7.18-7.2 (1H, m), 6.78-6.8 (2H, m), 5.9 (1H, br t), 3.84 (6H, s), 3.78 (3H, s), 3.63-3.69 (2H, m), 3.13-3.16 (1H, m), 3.07 (2H, t), 2.2 (3H, s), 1.27 (3H, s) and 1.25 (3H, s); m/z 503.15 (MH⁺)

### Example 26

### 2-(2-Bromo-5-tert-butylphenoxy)-N-(242-(isopropylamino)ethylamino)-4,6-dimethoxypyrimidin-5-yl)thiazole-4-carboxamide

Prepared from tert-butyl 2-(5-(2-(2-bromo-5-tert-butylphenoxy)thiazole-4-carboxamido)-4,6-dimethoxypyrimidin-2-ylamino)ethyl(isopropyl)carbamate (Intermediate 56; 0.48 g, 0.69 mmol) according to the protocol used to prepare Example 22. The title compound was isolated by column chromatography (Biotage KPNH, methanol: dichloromethane, 1:9) as an off-white foam (0.28 mg, 68%). ¹H NMR δ 7.88 (1H, s), 7.69 (1H, s), 7.56-7.58 (1H, m), 7.4 (1H, m), 7.2-7.23 (1H, m), 5.22 (1H, br t), 3.86 (6H, s), 3.47-3.52 (2H, m), 2.8-2.84 (3H, m), 1.3 (9H, s), 1.07 (3H, s) and 1.06 (3H, s); m/z 595.06 (MH⁺)

### Example 27

### 2-(5-tert-Butyl-2-methoxyphenoxy)-N-(2-(2-(isopropylamino)ethylamino)-4,6-dimethoxypyrimidin-5-yl)thiazole-4-carboxamide

Prepared from tert-butyl 2-(5-(2-(5-tert-butyl-2-methoxyphenoxy)thiazole-4-carboxamido)-4,6-dimethoxypyrimidin-2-ylamino)ethyl(isopropyl)carbamate (Intermediate 58, 0.53 g, 0.82 mmol) according to the procedure used to prepare Example 22. The title compound was isolated by column chromatography (Biotage KPNH, methanol: dichloromethane, 1:9) as a pale purple foam (0.44 g, 99%). ¹H NMR δ 7.92 (1H, s), 7.57 (1H, s), 7.22-7.26 (2H, m), 6.92-6.94 (1H, m), 5.17 (1H, br t), 3.82 (6H, s), 3.78 (3H, s), 3.44-3.48 (2H, m), 2.77-2.8 (3H, m), 1.27 (9H, s), 1.04 (3H, s) and 1.02 (3H, s); m/z 545.21 (MH⁺)

### Example 28

### 2-(5-Bromo-2-methylphenoxy)N-(2-2-(isopropylamino)ethylamino)-4,6-dimethoxypyrimidin-5-yl)thiazole-4-carboxamide

Prepared from tert-butyl 2-(5-(2-(5-bromc-2-methylphenoxy)thiazole-4-carboxamido)-4,6-dimethoxypyrimidin-2-ylamino)ethyl(isopropyl)carbamate (Intermediate 60, 0.35 g, 0.53 mmol) according to the procedure used to prepare Example 22. The title compound was isolated by column chromatography (Biotage aminopropyl, ethyl acetate: petroleum ether, 1:4) as an off-white foam (0.14 g, 48%). ¹H NMR δ 7.84 (1H, s), 7.68 (1H, s), 7.39 (1H, m), 7.31-7.33 (1H, m), 7.4-7.16 (1H, m), 5.33 (1H, br t), 3.85 (6H, s), 3.45-3.50 (2H, m), 2.78-2.82 (3H, m), 2.22 (3H, s), 1.06 (3H, s) and 1.04 (3H, s); m/z 553.08 (MH⁺)

### Example 29

### 2-(4-Chloro-5-isopropyl-2-methylphenoxy)-N-(2-(2-(isopropylamino)ethylamino)-4,6-dimethoxypyrimidin-5-yl)thiazole-4-carboxamide

Prepared from tert-butyl 2-(5-(2-(4-chloro-5-isopropyl-2-methylphenoxy)thiazole-4-carboxamido)-4,6-dimethoxypyrimidin-2-ylamino)ethyl(isopropyl)carbamate (Intermediate 61, 0.5 g, 0.77 mmol) according to the procedure described for Example 22. The title compound was isolated by column chromatography (Biotage aminopropyl, ethyl acetate:petroleum ether, 1:4) as an off-white foam (0.35 g, 83%). ¹H NMR δ 7.85 (1H, s), 7.64 (1H, s), 7.24 (1H, s), 7.16 (1H, s), 5.2 (1H, br t), 3.84 (6H, s), 3.45-3.5 (2H, m), 3.30-3.37 (1H, m), 2.77-2.83 (3H, m), 2.21 (3H, s), 1.21 (3H, s), 1.19 (3H, s), 1.06 (3H, s) and 1.04 (3H, s); m/z 549.24 (MH⁺).

### Example 30

### 2-(2-tert-Butylphenoxy)-N-(2-(2-(isopropylamino)ethylamino)-4,6-dimethoxypyrimidin-5-yl)thiazole-4-carboxamide

Prepared from tert-butyl 2-(5-(2-(2-tert-butylphenoxy)thiazole-4-carboxamido)-4,6-dimethoxypyrimidin-2-ylamino)ethyl(isopropyl)carbamate (Intermediate 63, 0.26 g, 0.5 mmol) according to the procedure described for Example 22. The title compound was isolated by column chromatography (C18; 5-95% acetonitrile in 0.05% ammonium hydroxide solution) as a pale cream glass (0.12 g, 47%). ¹H NMR δ 7.93 (1H, s), 7.69 (1H, s), 7.45-7.48 (1H, m), 7.21-7.27 (3H, m), 5.23 (1H, br t), 3.88 (6H, s), 3.49-3.53 (2H, m), 2.83-2.87 (3H, m), 1.41 (9H, s), 1.09 (3H, s) and 1.08 (3H, s); m/z 515.2 (MH⁺).

### Example 31

### N-(2-(2-(Isopropylamino)ethylamino)-4,6-dimethoxypyrimidin-5-yl)-2-(2-methyl-5-propoxyphenoxy)thiazole-4-carboxamide

Prepared from tert-butyl 2-(4,6-dimethoxy-5-(2-(2-methyl-5-propoxyphenoxy)thiazole-4-carboxamido)pyrimidin-2-ylamino)ethyl(isopropyl)carbamate (Intermediate 68, 0.35 g, 0.55 mmol) according to the procedure described for Example 22. The title compound was isolated by column chromatography (Biotage aminopropyl, ethyl acetate:petroleum ether, 1:4) as a pale cream glass (0.26 g, 88%). ¹H NMR δ 7.92 (1H, s), 7.65 (1H, s), 7.16-7.18 (1H, d), 6.77-6.79 (2H, m), 5.24 (1H, br t), 3.86-3.90 (2H, m), 3.87 (6H, s), 3.47-3.52 (2H, m), 2.79-2.85 (3H, m), 2.20 (3H, s), 1.75-1.82 (2H, m), 1.07 (3H, s), 1.06 (3H, s) and 1.02 (3H, t); m/z 531.20 (MH⁺).

### Example 32

### 2-(5-Isopropoxy-2-methylphenoxy)-N-(2-(2-(isopropylamino)ethylamino)-4,6-dimethoxypyrimidin-5-yl)thiazole-4-carboxamide

Prepared from tert-butyl 2-(5-(2-(5-isopropoxy-2-methylphenoxy)thiazole-4-carboxamido)-4,6-dimethoxypyrimidin-2-ylamino)ethyl(isopropyl)carbamate (Intermediate 73, 0.59 g, 0.94 mmol) according to the procedure described for Example 22. The title compound was isolated by column chromatography (Biotage aminopropyl, ethyl acetate: petroleum ether, 1:4) as a pale brown foam (0.26 g, 51%). ¹H NMR δ 7.91 (1H, s), 7.64 (1H, s), 7.16-7.18 (1H, m), 6.76-6.78 (2H, m), 5.24 (1H, br t), 4.45-4.51 (1H, m), 3.87 (6H, s), 3.47-3.52 (2H, m), 2.79-2.86 (3H, m), 2.2 (3H, s), 1.33 (3H, s), 1.31 (3H, s), 1.08 (3H, s) and 1.06 (3H, s); m/z 531.16 (MH⁺).

### Example 33

### 2-(5-Ethoxy-2-methylphenoxy)-N-(2-(2-(isopropylamino)ethylamino)-4,6-dimethoxypyrimidin-5-yl)thiazole-4-carboxamide

Prepared from tert-butyl 2-(5-(2-(5-ethoxy-2-methylphenoxy)thiazole-4-carboxamido)-4,6-dimethoxypyrimidin-2-ylamino)ethyl(isopropyl)carbamate (Intermediate 78; 0.57 g, 0.92 mmol) according to the procedure used for

### Example 22

The title compound was isolated by column chromatography (Biotage aminopropyl, ethyl acetate: petroleum ether, 1:4) as a pale purple foam (0.39 g, 82%). ¹H NMR δ 7.91 (1H, s), 7.64 (1H, s), 7.16-7.18 (1H, m), 6.76-6.79 (2H, m), 5.25 (1H, br t), 3.97-4.02 (2H, m), 3.86 (6H, s), 3.47-3.51 (2H, m), 2.80-2.85 (3H, m), 2.2 (3H, s), 1.39 (3H, t), 1.07 (3H, s) and 1.05 (3H, s); m/z 517.15 (MH⁺).

### Example 34

### 2-(5-Isobutoxy-2-ethylphenoxy)-N-(2-(2-(isopropylamino)ethylamino)-4,6-dimethoxypyrimidin-5-yl)thiazole-4-carboxamide

Prepared from tert-butyl 2-(5-(2-(5-isobutoxy-2-methylphenoxy)thiazole-4-carboxamido)-4,6-dimethoxypyrimidin-2-ylamino)ethyl(isopropyl)carbamate (Intermediate 83; 0.55 g, 0.85 mmol) according to the procedure described for Example 22. The title compound was isolated by column chromatography (Biotage aminopropyl, ethyl acetate: petroleum ether, 1:4) as a pale purple foam (0.31 g, 67%). ¹H NMR (CDCl₃) δ 7.91 (1H, s), 7.64 (1H, s), 7.16-7.18 (1H, m), 6.77-6.79 (2H, m), 5.25 (1H, br t), 3.86 (6H, s), 3.67-3.68 (2H, d), 3.47-3.51 (2H, m), 2.79-2.85 (3H, m), 2.2 (3H, s), 2.01-2.11 (1H, m), 1.07 (3H, s), 1.05 (3H, s), 1.01 (3H, s) and 0.99 (3H, s). m/z 545.18 (MH⁺).

### Example 35

### N-(2-(2-Hydroxyethylamino)-4,6-dimethoxypyrimidin-5-yl)-2-(2-methyl-5-(2-morpholinoethoxy)phenoxy)thiazole-4-carboxamide

The title compound was prepared from N-(2-(2-(tert-butyldimethylsilyloxy)ethylamino)-4,6-dimethoxypyrimidin-5-yl)-2-(2-methyl-5-(2-morpholinoethoxy)phenoxy)thiazole-4-carboxamide (Intermediate 87, 0.52 g, 0.77 mmol) by the same method as for Example 17. It was isolated as a pale pink foam (0.25 g, 58%). ¹H NMR δ 7.921 (1H, s), 7.68 (1H, s), 7.18-7.22 (1H, m), 6.79-6.81 (2H, m), 5.48 (1H, br t), 4.09 (2H, t), 3.87 (6H, s), 3.79 (2H, t), 3.71-3.73 (4H, m), 3.53-3.57 (2H, m), 2.79 (2H, t), 2.55-2.58 (4H, m) and 2.22 (3H, s); m/z 561.15 (MH⁺).

### Example 36

### 2-[(4,6-Dimethoxy-5-{[2-(3,3,6-trimethyl-indan-5-yloxy)-thiazole-4-carbonyl]-amino}-pyrimidin-2-ylamino)-methyl]-pyrrolidine-1-carboxylic acid tert-butyl ester

1-(3-Dimethylaminopropyl)-3-ethyl-carbodiimide hydrochloride (299 mg, 1.55 mmol) was added to a mixture of 2-(3,3,6-trimethyl-2,3-dihydro-1H-inden-5-yloxy)thiazole-4-carboxylic acid (Intermediate 24; 450 mg, 1.485 mmol), 2-[(5-amino-4,6-dimethoxy-pyrimidin-2-ylamino)-methyl]-pyrrolidine-1-carboxylic acid tert-butyl ester (Intermediate 88; 525 mg, 1.485 mmol), hydroxybenzotriazole (210 mg, 1.55 mmol) and triethylamine (0.418 ml, 2.97 mmol) in dichloromethane (20 ml). The solution was stirred for a further 18 hours at ambient temperature and then the mixture was washed with saturated aqueous sodium hydrogen carbonate solution (20 ml). The aqueous phase was back extracted (dichloromethane, 2×20 ml), the organic fractions combined, dried (MgSO₄) and the solvent was removed in vacuo. The title compound was isolated by column chromatography (SiO₂, elution 25% to 75% ethyl acetate in petroleum) to give the title compound as a tan gum (392 mg, 44%). ¹H NMR δ 7.96 (1H, s), 7.64 (1H, s), 7.13 (1H, s), 7.00 (1H, s), 5.67 (0.5H, s), 5.34 (0.5H, s), 4.14 (1H, q), 3.89 (6H, s), 2.90 (2H, t), 2.26 (3H, s), 1.98 (4H, t), 1.87 (2H, br s) and 1.52 (9H, m). ¹³C NMR δ 173.8, 166.2, 159.8, 152.7, 152.7, 144.2, 141.4, 127.6, 127.5, 116.99, 114.6, 53.9, 45.1, 44.2, 41.6, 29.7, 28.5, 28.5, 28.0, 23.9, 16.0; m/z 639.22 (MH⁺).

### Example 37

### N-(4,6-dimethoxy-2-(pyrrolidin-2-ylmethylamino)pyrimidin-5-yl)-2-(3,3,6-trimethyl-2,3-dihydro-1H-inden-5-yloxy)thiazole-4-carboxamide

2-[(4,6-Dimethoxy-5-{[2-(3,3,6-trimethyl-indan-5-yloxy)-hiazole-4-carbonyl]-amino}-pyrimidin-2-ylamino)-methyl]-pyrrolidine-1-carboxylic acid tert-butyl ester (Example 36; 355 mg, 0.55 mmol) was taken up in a mixture of trifluoroacetic acid (2 ml) and dichlromethane (18 ml). After 1 hour the reaction mixture was treated with saturated aqueous sodium hydrogen carbonate (50 ml) and the phases separated. The aqueous phase was back extracted (dichloromethane, 2×20 ml) and the organic phases combined, dried (MgSO₄) and the solvent was removed in vacuo. The title compound was isolated by column chromatography (Alumina, elution 5% to 10% methanol in DCM) to give the title compound (282 mg, 95%). ¹H NMR δ 7.97 (1H, s), 7.64 (1H, s), 7.13 (1H, s), 7.00 (1H, s), 5.29 (1H, t), 3.89 (6H, s), 3.54 (1H, m), 3.38 (1H, m), 3.28 (1H, m), 3.02-2.87 (4H, m), 2.26 (3H, s), 1.98 (4H, tr), 1.49 (2H, m) and 1.27 (6H, s). ¹³C NMR δ 173.8, 166.2, 159.8, 159.6, 152.7, 152.6, 144.1, 127.6, 127.5, 117.0, 114.6, 91.8, 58.1, 53.9, 46.5, 46.1, 44.1, 41.7, 29.7, 29.1, 28.5, 25.7 and 16.0; m/z 539.20 (MH⁺).

### Example 38

### tert-butyl 4-(4,6-dimethoxy-5-(2-(3,3,6-trimethyl-2,3-dihydro-1H-inden-5-yloxy)thiazole-4-carboxamido)pyrimidin-2-yl)-1,4-diazepane-1-carboxylate

1-(3-Dimethylaminopropyl)-3-ethyl-carbodiimide hydrochloride (299 mg, 1.55 mmol) was added to a solution of 2-(3,3,6-trimethyl-2,3-dihydro-1H-inden-5-yloxy)thiazole-4-carboxylic acid (Intermediate 24; 450 mg, 1.485 mmol), 4-(5-amino-4,6-dimethoxy-pyrimidin-2-yl)-[1,4]diazepane-1-carboxylic acid tert-butyl ester (Intermediate 90; 552 mg, 1.485 mmol), hydroxybenzotriazole (210 mg, 1.55 mmol) and triethylamine (0.418 ml, 2.97 mmol) in dichloromethane (20 ml). The solution was stirred for a further 18 hours at ambient temperature and then the mixture was washed with saturated aqueous sodium hydrogen carbonate solution (20 ml). The aqueous phase was back extracted (dichlrormethane, 2×20 ml), the organic fractions combined, dried (MgSO₄) and the solvent was removed in vacuo. The title compound was isolated by column chromatography (SiO₂, elution 25% to 75% ethyl acetate in petroleum) to give the title compound as a tan gum (455 mg, 48%). ¹H NMR δ 7.98 (1H, s), 7.64 (1H, s), 7.14 (1H, s), 7.00 (1H, s), 3.90 (6H, s), 3.86 (2H, m), 3.74 (2H, m), 3.55 (2H, t), 3.40 (1H, t), 3.32 (1H, t), 2.90 (2H, t), 2.26 (3H, s), 1.98 (4H, m), 1.48 (9H, d) and 1.27 (6H, s).

¹³C NMR δ 173.9, 166.0, 159.8, 157.9, 155.5, 155.2, 152.7, 144.2, 141.5, 127.6, 127.6, 117.0, 114.6, 53.8, 49.0, 48.0, 46.7, 46.6, 46.0, 44.1, 41.6, 31.0, 29.7, 28.5, 25.6, 16.0; m/z 638.75 (MH⁺).

### Example 39

### N-(2-(1,4-diazepan-1-yl)-4,6-dimethoxypyrimidin-5-yl)-2-(3,3,6-trimethyl-2,3-dihydro-1H-inden-5-yloxy)thiazole-4-carboxamide

tert-butyl 4-(4,6-dimethoxy-5-(2-(3,3,6-trimethyl-2,3-dihydro-1H-inden-5-yloxy)thiazole-4-carboxamido)pyrimidin-2-yl)-1,4-diazepane-1-carboxylate (Example 38; 419 mg, 0.65 mmol) was taken up in a mixture of trifluoroacetic acid (2 ml) and dichloromethane (18 ml). After 1 hour the reaction mixture was treated with saturated aqueous sodium hydrogen carbonate (50 ml) and the phases separated. The aqueous phase was back-extracted (dichlormethane, 2×20 ml) and the organic phases combined, dried (MgSO₄) and the solvent was removed in vacuo. The title compound was isolated by column chromatography (alumina, elution 5% to 10% methanol in dichlromethane) to give the title compound (282 mg, 99%). ¹H NMR δ 7.98 (1H, s), 7.64 (1H, s), 7.13 (1H, s), 7.01 (1H, s), 3.90 (6H, s), 3.84 (2H, m), 3.03 (2H, m), 2.90 (4H, m), 2.26 (3H, s), 1.99 (4H, m), 1.88 (2H, m) and 1.27 (6H, s). ¹³C NMR δ 173.8, 165.9, 159.8, 158.2, 152.7, 144.2, 141.5, 127.6, 127.5, 117.0, 114.7, 90.7, 53.7, 50.1, 48.9, 48.1, 46.5, 44.2, 41.7, 30.0, 29.7, 28.5 and 16.0; m/z 539.1 (MH⁺).

### Example 40

### tert-butyl 4-(4,6-dimethoxy-5-(2-(3,3,6-trimethyl-2,3-dihydro-1H-inden-5-yloxy)thiazole-4-carboxamido)pyrimidin-2-yl)piperazine-1-carboxylate

1-(3-Dimethylaminopropyl)-3-ethyl-carbodiimide hydrochloride (299 mg, 1.55 mmol) was added to a solution of 2-(3,3,6-trimethyl-2,3-dihydro-1H-inden-5-yloxy)thiazole-4-carboxylic acid (Intermediate 24; 450 mg, 1.485 mmol), 4-(5-Amino-4,6-dimethoxy-pyrimidin-2-yl)-piperazine-1-carboxylic acid tert-butyl ester (Intermediate 92; 548 mg, 1.485 mmol), hydroxybenzotriazole (210 mg, 1.55 mmol) and triethylamine (0.418 ml, 2.97 mmol) in dichloromethane (20 ml). The solution was stirred for a further 18 hours at ambient temperature and then the mixture was washed with saturated sodium hydrogen carbonate solution (20 ml). The aqueous phase was back extracted (dichloromethane, 2×20 ml), the organic fractions combined, dried (MgSO₄) and the solvent was removed in vacuo. The title compound was isolated by column chromatography (SiO₂, elution 25% to 75% ethyl acetate in petroleum) to give the title compound as a tan gum (606 mg, 65%). ¹H NMR δ 7.99 (1H, s), 7.65 (1H, s), 7.13 (1H, s), 7.00 (1H, s), 3.91 (6H, s), 3.79 (2H, m), 3.50 (2H, t), 2.90 (2H, t), 2.26 (3H, s), 1.98 (2H, t), 1.52 (9H, s) and 1.27 (6H, s). ¹³C NMR δ 173.9, 166.0, 159.8, 158.3, 154.8, 152.7, 144.1, 141.5, 127.6, 127.6, 117.0 and 114.6; m/z 624.75 (MH⁺).

### Example 41

### N-(4,6-dimethoxy-2-(piperazin-1-yl)pyrimidin-5-yl)-2-(3,3,6-trimethyl-2,3-dihydro-1H-inden-5-yloxy)thiazole-4-carboxamide

tert-butyl 4-(4,6-dimethoxy-5-(2-(3,3,6-trimethyl-2,3-dihydro-1H-inden-5-yloxy)thiazole-4-carboxamido)pyrimidin-2-yl)piperazine-1-carboxylate (Example 40; 545 mg, 0873 mmol) was taken up in a mixture of trifluoroacetic acid (2 ml) and dichlormethane (18 ml). After 1 hour the reaction mixture was treated with saturated aqueous sodium hydrogen carbonate (50 ml) and the phases separated. The aqueous phase was back extracted (dichlormethane, 2×20 ml) and the organic phases combined, dried (MgSO₄) and the solvent was removed in vacuo. The title compound was isolated by column chromatography (alumina, elution 5% to 10% methanol in dichloromethane) to give the title compound (363 mg, 100%). ¹H NMR δ 7.99 (1H, s), 7.64 (1H, s), 7.13 (1H, s), 7.01 (1H, s), 3.91 (6H, s), 3.80 (4H, m), 2.95-2.88 (6H, m), 2.27 (3H, s), 1.94 (4H, m) and 1.27 (6H, s). ¹³C NMR δ 173.8, 165.9, 159.8, 158.4, 152.68, 152.65, 144.2, 141.5, 127.6, 127.5, 117.0, 114.6, 91.0, 53.8, 46.0, 45.0, 46.5, 44.2, 41.7, 29.7, 29.3, 28.5 and 16.0; m/z 525.1 (MH⁺).

### Example 42

### 2-(5-(2-Hydroxypropan-2-yl)-2-methylphenoxy)-N-(2-(2-(isopropylamino)ethylamino)-4,6-dimethoxypyrimidin-5-yl)thiazole-4-carboxamide

The title compound was prepared according to the method outlined for Intermediate 1 from 5-(2-hydroxypropan-2-yl)-2-methylphenol (Indian Journal of Chemistry, 1984, 23B (II), 1098-1102), (0.332 g, 2 mmol) and 2-Bromo-N-(2-(2-(isopropylamino)ethylamino)-4,6-dimethoxypyrimidin-5-yl)thiazole-4-carboxamide (Intermediate 94; 0.410 g, 0.92 mmol). It was isolated as a pale orange foam (0.2959, 61%). ¹H NMR (d⁶-DMSO) δ 8.83 (1H, s), 7.75 (1H, s), 7.42-7.35 (3H, m), 7.07 (1H, m), 5.15 (1H, m), 3.78 (6H, s), 2.73-2.67 (3H, m), 2.21 (3H, s), 1.41 (6H, s), 0.96 (6H, d, J=6 Hz); m/z 531.6 (M+H⁺)

### Example 43

### 5-(3-tert-butylphenoxy)-N-(2-(2-(isopropylamino)ethylamino)-4,6-dimethoxypyrimidin-5-yl)-2-methyl-2H-1,2,4-triazole-3-carboxamide or 5-(3-tert-butylphenoxy)-N-(2-(2-(isopropylamino)ethylamino)-4,6-dimethoxypyrimidin-5-yl)-1-methyl-1H-1,2,4-triazole-3-carboxamide or 5-(3-tert-butylphenoxy)-N-(2-(2-(isopropylamino)ethylamino)-4,6-dimethoxypyrimidin-5-yl)-4-methyl-4H-1,2,4-triazole-3-carboxamide

Ethyl 5-bromo-4-methyl-4H-1,2,4-triazole-3-carboxylate and ethyl 5-bromo-2-methyl-2H-1,2,4-triazole-3-carboxylate and ethyl 5-bromo-1-methyl-1H-1,2,4-triazole-3-carboxylate (Intermediate 95; 0.345 g, 1.13 mmol) was dissolved in a mixture of THF (5 ml), water (5 ml) and lithium hydroxide (0.120 g, 5 mmol). The reaction was stirred for three hours before being diluted with water and acidified with 1 M hydrochloric acid. The mixture was extracted with ethyl acetate and the organic extract was dried (MgSO₄), filtered and evaporated to yield a solid, m/z 276 (MH)⁺, which was reacted with tert-butyl 2-(5-amino-4,6-dimethoxypyrimidin-2-yl amino)ethyl(isopropyl)carbamate (Intermediate 44) according to the method outlined for Intermediate 36 to yield the protected title compound [m/z 613 (MH)⁺]. This compound was dissolved in trifluoroacetic acid (5 ml) and stirred for one hour before evaporating the solvent and diluting with ethyl acetate. The organic layer was basified with saturated sodium carbonate solution and then dried (MgSO₄), filtered and evaporated. The title compound was isolated by column chromatography (Biotage KPNH, methanol: dichloromethane) as a white solid (0.275 g, 47%). NMR (DMSO) δ 8.81 (1H, s), 7.40-7.23 (4H, m), 6.99 (1H, t, J=6 Hz), 3.81 (3H, s), 3.75 (3H, s), 3.34-3.29 (3H, m), 2.75-2.66 (3H, m), 1.29 (9H, s), 0.96 (3H, d, J=6 Hz); m/z 513 (MH)⁺.

### Example 44

### 2-(5-tert-butyl-2-chlorophenoxy)-N-(2-(2-(isopropylamino)ethylamino)-4,6-dimethoxypyrimidin-5-yl)thiazole-4-carboxamide

tert-Butyl 2-(5-(2-(5-tert-butyl-2-chlorophenoxy)thiazole-4-carboxamido)-4,6-dimethoxypyrimidin-2-ylamino)ethyl(isopropyl)carbamate (intermediate 98, 9 mg, 0.38 mmol) was dissolved in dichloromethane (2 ml) and trifluoroacetic acid (1 ml) was added and the mixture was stirred at room temperature for 30 minutes. After this time the excess trifluoroacetic acid and dichloromethane were removed under reduced pressure and the resulting oil was dissolved in dichloromethane (10 ml) and saturated aqueous sodium bicarbonate solution (5 ml) were added. The layers were separated and the aqueous layer was extracted with dichloromethane (10 ml). The combined organic layers were dried (MgSO₄) and the solvent was removed under reduced pressure. The crude product was then purified column chromatography (aminopropyl cartridge, 0-15% methanol-dichloromethane) to give the product as a cream foam (64 mg, 31%). ¹H NMR δ 1.01 (6H, d), 1.33 (9H, s), 2.82 (t, 2H), 3.50 (q, 2H), 3.90 (s, 6H), 5.25 (t, 1H), 7.25 (1H, d), 7.40 (1H, d), 7.71 (1H, s), 7.88 (1H, s); m/z 549 (M⁺).

### Example 45

### 2-(5-tert-Butyl-2-methylphenoxy)-N-(4,6-dimethoxy-2-(morpholin-2-ylmethylamino)pyrimidin-5-yl)thiazole-4-carboxamide

tert-Butyl 2-((5-tert-butyl-2-methylphenoxy)thiazole-4-carboxamido)-4,6-dimethoxypyrimidin-2-ylamino)methyl)morpholine-4-carboxylate (intermediate 101, 130 mg, 0.2 mmol) was dissolved in dichloromethane (1 ml) and trifluouroacetic acid (0.5 ml) was added and the solution was stirred at room temperature for 30 minutes. After this time the excess trifluoroacetic acid and dichloromethane were removed under reduced pressure and the resulting oil was dissolved in dichloromethane (8 ml) and saturated aqueous sodium bicarbonate solution (4 ml) were added. The layers were separated and the aqueous layer was extracted with dichloromethane (5 ml). The combined organic layers were dried (MgSO₄) and the solvent was removed under reduced pressure. The crude product was then purified column chromatography (aminopropyl cartridge, 0-15% methanol-dichloromethane) to give the product as a pale cream foam (40 mg, 37%). ¹H NMR δ 1.28 (9H, s), 2.56-3.0 (4H, m), 3.31 (1H, m), 3.60-3.76 (4H, m), 3.90 (6H, s), 5.25 (1H, t), 7.22 (m, 3H), 7.66 (1, s); m/z 544 (MH⁺).

### Example 46

### N-(2-((1H-imidazol-2-yl)methylamino)-4,6-dimethoxypyrimidin-5-yl)-2-(5-tert-butyl-2-methylphenoxy)thiazole-4-carboxamide

Tetrabutylammonium fluoride (1 M in THF, 0.62 ml, 1.22 mmol) was added to a solution of 2-(5-tert-butyl-2-methylphenoxy)-N-(4,6-dimethoxy-2-((1-((2-(trimethylsilyl)ethoxy)methyl)-1H-imidazol-2-yl)methylamino)pyrimidin-5-yl)thiazole-4-carboxamide (Intermediate 110, 0.4 g, 0.61 mmol) in anhydrous THF (5 ml). The solution was refluxed (2 h) and then the material divided between ethyl acetate (30 ml) and water (30 ml). The organic layer was washed with brine (20 ml) and then dried (MgSO₄) and the solvent was removed in vacuo. The crude product was then purified by column chromatography (SiO₂; methanol-dichloromethane 1-19) to afford the title compound as a pale brown foam (0.17 g, 52%). ¹H NMR δ 7.98 (1H, br s), 7.65 (1H, s), 7.24-7.25 (3H, m), 6.97 (2H, s), 5.54 (1H, br t), 4.66-4.68 (2H, m), 3.89 (6H, s), 2.26 (3H, s) and 1.31 (9H, s); m/z 524.12 (MH⁺).

### Example 47

### 2-(5-tert-butyl-2-methylphenoxy)-N-(4,6-dimethoxy-2-(2-oxoimidazolidin-1-yl)pyrimidin-5-yl)thiazole-4-carboxamide

The title compound was prepared from 2-bromo-N-(4,6-dimethoxy-2-(2-oxoimidazolidin-1-yl)pyrimidin-5-yl)thiazole-4-carboxamide (Intermediate 115, 0.73 g, 1.3 mmol) and 5-tert-butyl-2-methylphenol (Intermediate 10, 0.42 g, 2.6 mmol) according to the procedure outlined for Intermediate 1. It was isolated as a pale brown solid (0.26 g, 44%). ¹H NMR δ 8.04 (1H, s), 7.65 (1H, s), 7.23-7.6 (3H, m), 5.29 (1H, s), 4.14 (2H, t), 3.99 (6H, s), 3.52 (2H, t), 2.26 (3H, s) and 1.31 (9H, s); m/z=513.15 (MH⁺).

### Example 48

### 2-(5-tert-butyl-2-cyanophenoxy)-N-(2-(2-(isopropylamino)-ethylamino)-4,6-dimethoxypyrimidin-5-yl)thiazole 4-carboxamide

tert-Butyl 2-(5-(2-(5-tert-butyl-2-cyanophenoxy)thiazole-4-carboxamido)-4,6-dimethoxypyrimidin-2-ylamino)ethyl(isopropyl)carbamate (Intermediate 117; 1.18 g, 1.85 mmol) was dissolved in anhydrous dichloromethane (10 ml) and TFA (2 ml, 20%) was added at room temperature. The reaction mixture was stirred for 2 hours before the solvent and TFA was evaporated under reduce pressure and the residue was dissolved in ethyl acetate and washed with a saturated aqueous solution of sodium bicarbonate and brine. The organic layer was dried (MgSO₄) and the solvent was evaporated to give the title compound (509 mg, 51%) after purification by column chromatography (ISOLUTE NH₂ Flash Chromatography cartridge; neat ethyl acetate). ¹H NMR 1.10 (6H, d, J=6.0 Hz), 1.36 (9H, s), 2.85 (3H, m), 3.51 (2H, q, J=6.0 Hz), 3.88 (6H, s), 3.90 (1H, m), 5.24 (1H, t, J=6.0 Hz), 7.41 (1H, dd, J=2.0 and 8.4 Hz), 7.62 (1H, d, J=2.0 Hz), 7.68 (1H, d, J=8.4 Hz), 7.76 (1H, s), 7.81 (1H, s), m/z=540 (MH⁺).

### Example 49

### N-(2-(2-(N-(bis(dimethylamino)methylene)sulfamoyl)ethylamino)-4,6-dimethoxypyrimidin-5-yl)-2-(5-tert-butyl-2-methyl-phenoxy)thiazole-4-carboxamide

2-(5-tert-Butyl-2-methylphenoxy)thiazole-4-carboxylic acid (Intermediate 18; 90 mg, 0.31 mmol) and 2-(5-amino-4,6-dimethoxypyrimidin-2-ylamino)-ethanesulfonamide (Intermediate 119; 86 mg, 0.31 mmol) were dissolved in dichloromethane (2 ml) and triethylamine (0.09 ml, 0.61 mmol) and HBTU (171 mg, 0.45 mmol) were added at room temperature. The reaction was stirred until complete (LC-MS) and then saturated aqueous sodium bicarbonate solution was added and the solvent was evaporated. The residue was extracted into ethyl acetate, washed with water and brine, and dried (MgSO₄). Evaporation of the solvent followed by purification by preparative HPLC (LCMSXTERRAMETHOD; injection volume: 50 μL, retention time: 8.37 minutes) afforded the title compound (17 mg, 9%).

¹H NMR 1.34 (9H, s), 2.29 (3H, s), 3.01 (12H, s), 3.34 (2H, t, J=6.4 Hz), 3.89 (6H, s), 3.99 (2H, q, J=6.4 Hz), 5.88 (1H, t, J=6.4 Hz), 7.26 (3H, m), 7.65 (1H, s), 7.94 (1H, s); m/z=649 (MH⁺).

### Example 50

### 2-(5-tert-butyl-2-methylphenoxy)-N-(2-(2-(N-isopropylsulfamoyl)ethylamino)-4,6-dimethoxypyrimidin-5-yl)thiazole-4-carboxamide

2-(5-tert-Butyl-2-methylphenoxy)thiazole-4-carboxylic acid (Intermediate 18; 48 mg, 0.16 mmol) and 2-(5-amino-4,6-dimethoxypyrimidin-2-ylamino)-N-isopropylethanesulfonamide (Intermediate 123; 52 mg, 0.16 mmol) were dissolved in dichloromethane (3 ml) and triethylamine (0.05 ml, 0.32 mmol) and HBTU (91 mg, 0.24 mmol) were added at room temperature. The reaction was stirred until complete (LC-MS). Then saturated aqueous sodium bicarbonate solution was added and the solvent was evaporated. The residue was extracted into ethyl acetate, washed with water and brine, and dried (MgSO₄). Evaporation of the solvent followed by purification by preparative HPLC (LCMSXTERRAMETHOD; injection volume 150 μL; retention time 11.15 minutes) afforded the title compound (10 mg, 11%).

¹HNMR (CD₃OD): 1.23 (6H, d, J=6.4 Hz), 1.34 (9H, s), 2.26 (3H, s), 3.37 (3H, m), 3.56 (1H, m), 3.83 (2H, t, J=6.0 Hz), 3.91 (6H, s), 7.32 (3H, m), 7.72 (1H, s); m/z=593 (M+H⁺).

### Example 51

### 2-(5-tert-butyl-2-methylphenoxy)-N-(2-(2-cyanoethylamino)-4,6-dimethoxypyrimidin-5-yl)thiazole-4-carboxamide

2-(5-tert-Butyl-2-methylphenoxy)thiazole-4-carboxylic acid (Intermediate 18; 228 mg, 0.78 mmol) and 3-(5-amino-4,6-dimethoxypyrimidin-2-ylamino)propanenitrile (Intermediate 125; 175 mg, 0.78 mmol) were dissolved in dichloromethane (7 ml) and triethylamine (0.28 mL, 2.0 mmol) and HBTU (569 mg, 1.5 mmol) were added at room temperature. The reaction was stirred until complete (LC-MS). Saturated aqueous sodium bicarbonate solution was added and the solvent was evaporated before the residue was extracted into ethyl acetate, washed with water and brine and dried (MgSO₄). Evaporation of the solvent followed by crystallization from methanol afforded the title compound (47 mg, 12%). ¹H NMR 1.34 (9H, s), 2.29 (3H, s), 2.76 (2H, t, J=6.8 Hz), 3.72 (2H, q, J=6.8 Hz), 3.91 (6H, s), 5.29 (1H, t, J=6.8 Hz), 7.26 (3H, m), 7.66 (1H, s), 7.96 (1H, s); m/z=497 (M+H⁺).

### Example 52

### 3-(5-(2-(5-tert-butyl-2-methylphenoxy)thiazole-4-carboxamido)-4,6-dimethoxypyrimidin-2-ylamino)propanoic acid

Ethyl 3-(5-(2-(5-tert-butyl-2-methylphenoxy)thiazole-4-carboxamido)-4,6-dimethoxypyrimidin-2-ylamino)propanoate (Intermediate 128; 220 mg, 0.40 mmol) was dissolved in THF (4 ml) and a solution aqueous sodium hydroxide (0.2 M, 2 mL) was added at room temperature. The mixture was heated to 45° C. for 3 hours. After allowing the reaction to cool to room temperature, hydrochloric acid (0.2 M) was added and the reaction was extracted into ethyl acetate and washed with water and brine. The organic layer was dried (MgSO₄) and the solvent was evaporated to afford the final compound (43 mg, 21%) after purification in the preparative HPLC (LCMSXTERRAMETHOD; injection volume 300 μL; retention time 8.37 minutes). ¹H NMR 1.24 (9H, s), 2.19 (3H, s), 2.54 (2H, t, J=6.0 Hz), 3.63 (2H, q, J=6.0 Hz), 3.82 (6H, s), 5.76 (1H, t, J=6.0 Hz), 7.17 (3H, m), 7.59 (1H, s), 7.93 (1H, s); m/z=516 (M+H⁺).

### Example 53

### Human GnRH Receptor Functional Assay

GnRH-receptor antagonists may be functionally assessed by measurement of change in intracellular calcium levels induced by Gαq mediated increase in IP3 levels. The ability of compounds to block the intracellular release of calcium by GnRH in CHO-K1 cells expressing human GnRH receptors is determined as a measure of the compound's antagonist activity in vitro. Approximately 30,000 cells per assay well (half well 96 well assay plate—Corning) are seeded in normal culture medium. Twenty four hours after seeding the cells are loaded with a calcium sensitive fluorescent dye by replacing the culture medium with assay buffer (1× Hanks buffered saline, 25 mM HEPES, 0.1% w/v fatty acid free BSA, pH7.4) containing 2.5 mM probenecid and 1× Calcium Plus reagent (Molecular) Devices. Cells are incubated at 37° C. for 1 hour to allow for dye uptake. To test for antagonist activity, compounds at a concentration range between 0.1 nM-3.2 μM are added to the assay wells and allowed to incubate 20 minutes prior to stimulation with GnRH. After incubation with test compounds the assay plate is placed in a Flexstation II (Molecular Devices) and GnRH is added at the determined EC₈₀ concentration (final). Ligand-dependent changes in intracellular calcium levels are determined by measuring changes in fluorescence of the dye at 525 nM following excitation at 485 nM. Percentage inhibition curves are plotted using 4-parameter fit algorithm and IC₅₀ values calculated for each compound.

| | | Human |
|---|---|---|
| Example | | GnRH |
| Number | Compound Name | IC₅₀ (nM) |
| 2 | 2-(3-tert-butylphenoxy)-N-(4,6-dimethoxy-2-(3- | 485 |
| | morpholinopropylamino)pyrimidin-5-yl)thiazole-4- | |
| | carboxamide. | |
| 5 | 2-(5-tert-Butyl-2-methylphenoxy)-N-(2-(2- | 193 |
| | (dimethylamino)ethylamino)-4,6-dimethoxypyrimidine-5- | |
| | yl)thiazole-4-carboxamide. | |
| 24 | N-(2-(2-(Isopropylamino)ethylamino)-4,6- | 4 |
| | dimethoxypyrimidin-5-yl)-2-(6-methoxy-3,3-dimethyl-2,3- | |
| | dihydro-1H-inden-5-yloxy)thiazole-4-carboxamide | |

1. A compound of formula (I): 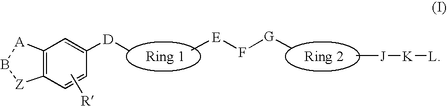
wherein
either B is absent and A and Z are the same or different and are each hydrogen, halogen, alkyl, hydroxy, alkoxy, —ON, —C(R^{c})₂OH, —N(R^{d})C(═X)R^{c}, —C(═X)N(R^{c})(R^{d}), —S(O)ₘ—R^{c}, —N(R^{c})(R^{d})S(O)₂, —S(O)₂N(R^{c})(R^{d}), —N(R^{e})₂, aryl optionally substituted with R^{a} or —O-aryl optionally substituted with R^{a}; or
B is present and is —(CH₂)ₙ—, —C(R^{b})₂— or —O—, or B taken together with A or Z can be —C═C(R^{b})—, —C(R^{b})═C—, —CH₂—CH(R^{b})— or —CH(R^{b})—CH₂—;
D is —O— or —S(O)ₘ—;
E is a bond or is —(CH₂)ₙ—, —N(R^{d})—, —(CH₂)ₙN(R^{d})— or N(R^{d})(CH₂)ₙ—;
F is —C(═X)—;
G is —(CH₂)ₙ—, —N(R^{d})—, —(CH₂)ₙN(R^{d})— or N(R^{d})(CH₂)ₙ;
J is a bond or is —O—, —N(R^{c})C(═X)—, —C(═X)N(R^{c})—, —S(O)ₘ—, —N(R^{c})S(O)ₘ—, —S(O)ₘN(R^{c})—, —N(R^{e})— or —N(R^{g})(R^{h});
K is a bond or is alkylene optionally substituted with R^{b}; or K is cycloalkylene, cycloalkenylene, arylene, heterocycloalkylene, heterocycloalkylene or heteroarylene, any of which is optionally substituted with R^{a};
L is hydrogen, halogen, —N(R^{f})₂, —CN, —SO₂N(R^{a})₂, —SO₂N═C[N(R^{a})₂], —NHC(═O)NHOR^{a}, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}), —CN or a group of the formula 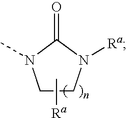
each R^{a} is the same or different and is hydrogen, halogen, alkyl, aryl, hydroxy, alkoxy, -alkoxy-(CH₂)ₙC(═O)OR^{b}, —O-aryl, —C(═X)R^{c}, —NO₂, —CN, —N(R^{c})C(═X)R^{c}, —C(═X)N(R^{c})₂, —S(O)₂N(R^{c})₂ or —N(R^{e})₂;
each R^{b} is the same or different and is hydrogen or alkyl;
each R^{c} is the same or different and is alkyl, cycloalkyl, -alkyl-aryl, -alkyl-cycloalkyl or aryl optionally substituted with R^{a};
each R^{d} is the same or different and is hydrogen or alkyl or aryl optionally with R^{a};
each R^{e} is the same or different and is hydrogen or alkyl; or R^{e} is aryl or heteroaryl, either of which is optionally substituted with R^{a};
each R^{f} is the same or different and is hydrogen or alkyl; or R^{f}—N—R^{f} taken together forms heterocycloalkyl, heterocycloalkenyl or heteroaryl, each of which is optionally substituted with R^{e};
each R^{g} is alkyl, cycloalkyl or alkyl-cycloalkyl, any of which is optionally substituted by an oxo and/or fluoro group;
each R^{h} is alkyl, cycloalkyl, or alkyl-cycloalkyl substituted with N(R^{f})₂;
or R^{g} and R^{h} are taken together to form a heterocycloalkyl ring;
each X is the same or different and is oxygen or sulphur;
Ring 1 is a five- or six-membered heteroaryl ring containing at least 2 heteroatoms from O, N and/or S, which is optionally substituted with one or more R^{a};
Ring 2 is arylene or heteroarylene, either of which is optionally substituted with one or more R^{a};
each m is the same or different and is 0, 1 or 2; and
each n is the same or different and is 0, 1, 2 or 3;
or a pharmaceutically acceptable salt thereof.

2. The compound according to claim 1, wherein D is —O— or —S—.

3. The compound according to claim 1, wherein E is absent.

4. The compound according to claim 1, wherein F is —C(O)—.

5. The compound according to claim 1, wherein G is —N(R^{d})—.

6. The compound according to claim 5, wherein R^{d} is hydrogen.

7. The compound according to claim 1 wherein J is —N(R^{e}) or —O—.

8. The compound according to claim 1 wherein K is ethylene or propylene.

9. The compound according to claim 1, wherein Ring 2 is phenylene, pyrimidylene or pyridinylene, any of which is optionally substituted.

10. The compound according to claim 10, wherein Ring 2 is substituted 1, 2 or 3 times, the substituents being the same or different and selected from alkoxy, halogen and J-K-L.

11. The compound according to any preceding claim 1, wherein either B is absent and A and Z are the same or different and are each hydrogen, halogen, alkyl, hydroxy, alkoxy, —CN, —N(R^{d})C(═X)R^{c}, —C(═X)N(R^{c})(R^{d}), —S(O)ₘ—R^{c}, —N(R^{c})(R^{d})S(O)₂, —S(O)₂N(R^{c})(R^{d}), —N(R^{e})₂, aryl optionally substituted with R^{a} or —O-aryl optionally substituted with R^{a}; or
B is present and is —(CH₂)ₙ—, —C(R^{b})₂— or —O—, or B taken together with A or Z can be —C═C(R^{b})—, —C(R^{b})═C—, —CH₂—CH(R^{b})— or —CH(R^{b})—CH₂—;
J is a bond or is —O—, —N(R^{c})C(═X)—, —C(═X)N(R^{c})—, —S(O)ₘ—, —N(R^{c})S(O)ₘ—, —S(O)ₘN(R^{c})—, —N(R^{e})— or —N(R^{g})(R^{h});
L is hydrogen, halogen, —N(R^{f})₂, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}) or —CN; and
Ring 1 is a five- or six-membered heteroaryl ring containing at least 2 heteroatoms from O, N and S.

12. The compound according to any preceding claim 1, wherein Ring 1 is oxazole, thiazole or triazole.

13. The compound according to claim 11, wherein Ring 1 is oxazole or thiazole.

14. The compound according to claim 1, selected from:
2-(5-tert-butyl-2-methylphenoxy)-N-(2-(2-(dimethylamino)ethylamino)-4,6-dimethoxypyrimidin-5-yl)oxazole-4-carboxamide;
2-(5-tert-butyl-2-methylphenoxy)-N-(2-(2-(dimethylamino)ethylamino)-4,6-dimethoxypyrimidin-5-yl)thiazole-4-carboxamide;
2-(5-tert-butyl-2-methylphenoxy)-N-(2-(dimethylamino)-4,6-dimethoxypyrimidin-5-yl)oxazole-4-carboxamide;
2-(5-tert-butyl-2-methyl phenoxy)-N-(2-(dimethylamino)-4,6-dimethoxypyrimidin-5-yl)thiazole-4-carboxamide.
2-(5-tert-butyl-2-methylphenoxy)-N-(4,6-dimethoxy-2-(1-methylazetidin-3-ylamino)pyrimidin-5-yl)oxazole-4-carboxamide;
2-(5-tert-butyl-2-methylphenoxy)-N-(4,6-dimethoxy-2-(1-methylazetidin-3-ylamino)pyrimidin-5-yl)thiazole-4-carboxamide;
2-(3-tert-butylphenoxy)-N-(2-(2-(dimethylamino)ethylamino)-4,6-dimethoxypyrimidin-5-yl)oxazole-4-carboxamide;
2-(3-tert-butylphenoxy)-N-(2-(2-(dimethylamino)ethylamino)-4,6-dimethoxypyrimidin-5-yl)thiazole-4-carboxamide;
2-(3-tert-butyl phenoxy)-N-(2-(dimethylamino)-4,6-dimethoxypyrimidin-5-yl)oxazole-4-carboxamide;
2-(3-tert-butylphenoxy)-N-(2-(dimethylamino)-4,6-dimethoxypyrimidin-5-yl)thiazole-4-carboxamide;
2-(3-tert-butylphenoxy)-N-(4,6-dimethoxy-2-(1-methylazetidin-3-ylamino)pyrimidin-5-yl)oxazole-4-carboxamide;
2-(3-tert-butyl phenoxy)-N-(4,6-dimethoxy-2-(1-methylazetidin-3-ylamino)pyrimidin-5-yl)thiazole-4-carboxamide;
2-(3-tert-butyl phenoxy)-N-(4,6-dimethoxy-2-(3-morpholinopropylamino)pyrimidin-5-yl)oxazole-4-carboxamide;
2-(3-tert-butylphenoxy)-N-(4,6-dimethoxy-2-(3-morpholinopropylamino)pyrimidin-5-yl)thiazole-4-carboxamide;
2-(3,3-dimethyl-2,3-dihydro-1H-inden-5-yloxy)-N-(2-(2-(dimethylamino)ethylamino)-4,6-dimethoxypyrimidin-5-yl)oxazole-4-carboxamide;
2-(3,3-dimethyl-2,3-dihydro-1H-inden-5-yloxy)-N-(2-(2-(dimethylamino)ethylamino)-4,6-dimethoxypyrimidin-5-yl)thiazole-4-carboxamide;
2-(3,3-dimethyl-2,3-dihydro-1H-inden-5-yloxy)-N-(2-(dimethylamino)-4,6-dimethoxypyrimidin-5-yl)oxazole-4-carboxamide;
2-(3,3-dimethyl-2,3-dihydro-1H-inden-5-yloxy)-N-(2-(dimethylamino)-4,6-dimethoxypyrimidin-5-yl)thiazole-4-carboxamide;
N-(4,6-dimethoxy-2-(1-methylazetidin-3-ylamino)pyrimidin-5-yl)-2-(3,3-dimethyl-2,3-dihydro-1H-inden-5-yloxy)oxazole-4-carboxamide;
N-(4,6-dimethoxy-2-(1-methylazetidin-3-ylamino)pyrimidin-5-yl)-2-(3,3-dimethyl-2,3-dihydro-1H-inden-5-yloxy)thiazole-4-carboxamide;
N-(2-(2-(dimethylamino)ethylamino)-4,6-dimethoxypyrimidin-5-yl)-2-(3,3,6-trimethyl-2,3-dihydro-1H-inden-5-yloxy)oxazole-4-carboxamide;
N-(2-(2-(dimethylamino)ethylamino)-4,6-dimethoxypyrimidin-5-yl)-2-(3,3,6-trimethyl-2,3-dihydro-1H-inden-5-yloxy)thiazole-4-carboxamide;
N-(2-(dimethylamino)-4,6-dimethoxypyrimidin-5-yl)-2-(3,3,6-trimethyl-2,3-dihydro-1H-inden-5-yloxy)oxazole-4-carboxamide;
N-(2-(dimethylamino)-4,6-dimethoxypyrimidin-5-yl)-2-(3,3,6-trimethyl-2,3-dihydro-1H-inden-5-yloxy)thiazole-4-carboxamide;
N-(4,6-dimethoxy-2-(1-methylazetidin-3-ylamino)pyrimidin-5-yl)-2-(3,3,6-trimethyl-2,3-dihydro-1H-inden-5-yloxy)oxazole-4-carboxamide;
N-(4,6-dimethoxy-2-(1-methylazetidin-3-ylamino)pyrimidin-5-yl)-2-(3,3,6-trimethyl-2,3-dihydro-1H-inden-5-yloxy)thiazole-4-carboxamide;
2-(5-tert-butyl-2-methylphenoxy)-N-(4,6-dimethoxy-2-(methylamino)pyrimidin-5-yl)oxazole-4-carboxamide;
2-(5-tert-butyl-2-methylphenoxy)-N-(2-(3-(dimethylamino)propylamino)-4,6-dimethoxypyrimidin-5-yl)oxazole-4-carboxamide;
N-(2-(2-aminoethylamino)-4,6-dimethoxypyrimidin-5-yl)-2-(5-tert-butyl-2-methylphenoxy)oxazole-4-carboxamide;
2-(5-tert-butyl-2-methyl phenoxy)-N-(4,6-dimethoxy-2-(2-(methylamino)ethylamino)pyrimidin-5-yl)oxazole-4-carboxamide;
2-(5-tert-butyl-2-methylphenoxy)-N-(2-(2-(ethyl(methyl)amino)ethylamino)-4,6-dimethoxypyrimidin-5-yl)oxazole-4-carboxamide;
N-(2-(2-(azetidin-1-yl)ethylamino)-4,6-dimethoxypyrimidin-5-yl)-2-(5-tert-butyl-2-methylphenoxy)oxazole-4-carboxamide;
2-(5-tert-butyl-2-methylphenoxy)-N-(4,6-dimethoxy-2-(2-(pyrrolidin-1-yl)ethylamino)pyrimidin-5-yl)oxazole-4-carboxamide;
2-(5-tert-butyl-2-methyl phenoxy)-N-(4,6-dimethoxy-2-(3-(pyrrolidin-1-yl)propylamino)pyrimidin-5-yl)oxazole-4-carboxamide;
2-(5-tert-butyl-2-methylphenoxy)-N-(2-((2-(dimethylamino)ethyl)(methyl)amino)-4,6-dimethoxypyrimidin-5-yl)oxazole-4-carboxamide;
2-(5-tert-butyl-2-methyl phenoxy)-N-(2-((2-(trimethylammonium)ethyl)amino)-4,6-dimethoxypyrimidin-5-yl)oxazole-4-carboxamide iodide;
2-(5-tert-butyl-2-methylphenoxy)-N-(4,6-dimethoxy-2-(1-methylpiperidin-4-ylamino)pyrimidin-5-yl)oxazole-4-carboxamide;
2-(5-tert-butyl-2-methylphenoxy)-N-(4,6-dimethoxy-2-(piperidin-4-ylamino)pyrimidin-5-yl)oxazole-4-carboxamide;
2-(5-tert-butyl-2-methylphenoxy)-N-(4,6-dimethoxy-2-(1-methylpiperidin-4-yloxy)pyrimidin-5-yl)oxazole-4-carboxamide;
2-(5-tert-butyl-2-methylphenoxy)-N-(4,6-dimethoxy-2-(piperidin-4-yloxy)pyrimidin-5-yl)oxazole-4-carboxamide;
N-(2-(azetidin-3-ylamino)-4,6-dimethoxypyrimidin-5-yl)-2-(5-tert-butyl-2-methylphenoxy)oxazole-4-carboxamide;
2-(5-tert-butyl-2-methylphenoxy)-N-(4,6-dimethoxy-2-(1-methylazetidin-3-yloxy)pyrimidin-5-yl)oxazole-4-carboxamide;
N-(2-(azetidin-3-yloxy)-4,6-dimethoxypyrimidin-5-yl)-2-(5-tert-butyl-2-methyl phenoxy)oxazole-4-carboxamide;
N-(2-(3-aminopyrrolidin-1-yl)-4,6-dimethoxypyrimidin-5-yl)-2-(5-tert-butyl-2-methyl phenoxy)oxazole-4-carboxamide;
2-(5-tert-butyl-2-methylphenoxy)-N-(2-((1-ethylpyrrolidin-2-yl)methylamino)-4,6-dimethoxypyrimidin-5-yl)oxazole-4-carboxamide;
2-(5-tert-butyl-2-methylphenoxy)-N-(2-((pyrrolidin-2-yl)methylamino)-4,6-dimethoxypyrimidin-5-yl)oxazole-4-carboxamide;
2-(5-tert-butyl-2-methylphenoxy)-N-(2-(2-hydroxyethylamino)-4,6-dimethoxypyrimidin-5-yl)oxazole-4-carboxamide;
2-(5-tert-butyl-2-methylphenoxy)-N-(2-(2-hydroxyethylamino)-4,6-dimethoxypyrimidin-5-yl)thiazole-4-carboxamide;
N-(2-(2-aminoethylamino)-4,6-dimethoxypyrimidin-5-yl)-2-(5-tert-butyl-2-methylphenoxy)thiazole-4-carboxamide;
2-(5-tert-butyl-2-methylphenoxy)-N-(4,6-dimethoxy-2-(2-(methylamino)ethylamino)pyrimidin-5-yl)thiazole-4-carboxamide;
2-(5-tert-butyl-2-methylphenoxy)-N-(2-(2-(ethylamino)ethylamino)-4,6-dimethoxypyrimidin-5-yl)oxazole-4-carboxamide;
2-(5-tert-butyl-2-methylphenoxy)-N-(2-(2-(ethylamino)ethylamino)-4,6-dimethoxypyrimidin-5-yl)thiazole-4-carboxamide;
2-(5-tert-butyl-2-methyl phenoxy)-N-(2-(2-(isopropylamino)ethylamino)-4,6-dimethoxypyrimidin-5-yl)oxazole-4-carboxamide;
2-(5-tert-butyl-2-methylphenoxy)-N-(2-(2-(isopropylamino)ethylamino)-4,6-dimethoxypyrimidin-5-yl)thiazole-4-carboxamide;
2-(5-tert-butyl-2-methylphenoxy)-N-(2-(2-(tert-butylamino)ethylamino)-4,6-dimethoxypyrimidin-5-yl)oxazole-4-carboxamide;
2-(5-tert-butyl-2-methylphenoxy)-N-(2-(2-(tert-butylamino)ethylamino)-4,6-dimethoxypyrimidin-5-yl)thiazole-4-carboxamide;
N-(2-(2-hydroxyethylamino)-4,6-dimethoxypyrimidin-5-yl)-2-(5-isopropyl-2-methylphenoxy)oxazole-4-carboxamide;
N-(2-(2-hydroxyethylamino)-4,6-dimethoxypyrimidin-5-yl)-2-(5-isopropyl-2-methylphenoxy)thiazole-4-carboxamide;
N-(2-(2-aminoethylamino)-4,6-dimethoxypyrimidin-5-yl)-2-(5-isopropyl-2-methylphenoxy)oxazole-4-carboxamide;
N-(2-(2-aminoethylamino)-4,6-dimethoxypyrimidin-5-yl)-2-(5-isopropyl-2-methyl phenoxy)thiazole-4-carboxamide;
N-(4,6-dimethoxy-2-(2-(methylamino)ethylamino)pyrimidin-5-yl)-2-(5-isopropyl-2-methylphenoxy)oxazole-4-carboxamide;
N-(4,6-dimethoxy-2-(2-(methylamino)ethylamino)pyrimidin-5-yl)-2-(5-isopropyl-2-methylphenoxy)thiazole-4-carboxamide;
N-(4,6-dimethoxy-2-(2-(ethylamino)ethylamino)pyrimidin-5-yl)-2-(5-isopropyl-2-methylphenoxy)oxazole-4-carboxamide;
N-(4,6-dimethoxy-2-(2-(ethylamino)ethylamino)pyrimidin-5-yl)-2-(5-isopropyl-2-methylphenoxy)thiazole-4-carboxamide;
2-(5-isopropyl-2-methylphenoxy)-N-(2-(2-(isopropylaminoethylamino) 4,6-dimethoxypyrimidin-5-yl)oxazole-4-carboxamide;
2-(5-isopropyl-2-methylphenoxy)-N-(2-(2-(isopropylamino)ethylamino)-4,6-dimethoxypyrimidin-5-yl)thiazole-4-carboxamide;
N-(2-(2-(tert-butylamino)ethylamino)-4,6-dimethoxypyrimidin-5-yl)-2-(5-isopropyl-2-methylphenoxy)oxazole-4-carboxamide;
N-(2-(2-(tert-butylamino)ethylamino)-4,6-dimethoxypyrimidin-5-yl)-2-(5-isopropyl-2-methylphenoxy)thiazole-4-carboxamide;
N-(2-(2-hydroxyethylamino)-4,6-dimethoxypyrimidin-5-yl)-2-(3,3,6-trimethyl-2,3-dihydro-1H-inden-5-yloxy)oxazole-4-carboxamide;
N-(2-(2-hydroxyethylamino)-4,6-dimethoxypyrimidin-5-yl)-2-(3,3,6-trimethyl-2,3-dihydro-1H-inden-5-yloxy)thiazole-4-carboxamide;
N-(2-(2-aminoethylamino)-4,6-dimethoxypyrimidin-5-yl)-2-(3,3,6-trimethyl-2,3-dihydro-1H-inden-5-yloxy)oxazole-4-carboxamide;
N-(2-(2-aminoethylamino)-4,6-dimethoxypyrimidin-5-yl)-2-(3,3,6-trimethyl-2,3-dihydro-1H-inden-5-yloxy)thiazole-4-carboxamide;
N-(4,6-dimethoxy-2-(2-(methylamino)ethylamino)pyrimidin-5-yl)-2-(3,3,6-trimethyl-2,3-dihydro-1H-inden-5-yloxy)oxazole-4-carboxamide;
N-(4,6-dimethoxy-2-(2-(methylamino)ethylamino)pyrimidin-5-yl)-2-(3,3,6-trimethyl-2,3-dihydro-1H-inden-5-yloxy)thiazole-4-carboxamide;
N-(2-(2-(ethylamino)ethylamino)-4,6-dimethoxypyrimidin-5-yl)-2-(3,3,6-trimethyl-2,3-dihydro-1H-inden-5-yloxy)oxazole-4-carboxamide;
N-(2-(2-(ethylamino)ethylamino)-4,6-dimethoxypyrimidin-5-yl)-2-(3,3,6-trimethyl-2,3-dihydro-1H-inden-5-yloxy)thiazole-4-carboxamide;
N-(2-(2-(isopropylamino)ethylamino)-4,6-dimethoxypyrimidin-5-yl)-2-(3,3,6-trimethyl-2,3-dihydro-1H-inden-5-yloxy)oxazole-4-carboxamide;
N-(2-(2-(isopropylamino)ethylamino)-4,6-dimethoxypyrimidin-5-yl)-2-(3,3,6-trimethyl-2,3-dihydro-1H-inden-5-yloxy)thiazole-4-carboxamide;
N-(2-(2-(tert-butylamino)ethylamino)-4,6-dimethoxypyrimidin-5-yl)-2-(3,3,6-trimethyl-2,3-dihydro-1H-inden-5-yloxy)oxazole-4-carboxamide;
N-(2-(2-(tert-butylamino)ethylamino)-4,6-dimethoxypyrimidin-5-yl)-2-(3,3,6-trimethyl-2,3-dihydro-1H-inden-5-yloxy)thiazole-4-carboxamide;
2-(5-tert-butyl-2-methyl phenoxy)-N-(4,6-dimethoxy-2-(2-oxoimidazolidin-1-yl)pyrimidin-5-yl)oxazole-4-carboxamide;
2-(5-tert-butyl-2-methylphenoxy)-N-(4,6-dimethoxy-2-(2-oxoimidazolidin-1-yl)pyrimidin-5-yl)thiazole-4-carboxamide;
N-(2-(2-tert-butoxyethoxy)-4,6-dimethoxypyrimidin-5-yl)-2-(5-tert-butyl-2-methyl phenoxy)thiazole-4-carboxamide;
2-(5-tert-butyl-2-methyl phenoxy)-N-(2-(2-hydroxyethoxy)-4,6-dimethoxypyrimidin-5-yl)thiazole-4-carboxamide;
2-(5-chloro-2-methylphenoxy)-N-(2-(2-hydroxyethylamino)-4,6-dimethoxypyrimidin-5-yl)thiazole-4-carboxamide;
2-(2,5-dimethylphenoxy)-N-(2-(2-hydroxyethylamino)-4,6-dimethoxypyrimidin-5-yl)thiazole-4-carboxamide;
N-(2-(2-hydroxyethylamino)-4,6-dimethoxypyrimidin-5-yl)-2-(o-tolyloxy)thiazole-4-carboxamide;
2-(2-chloro-5-(trifluoromethyl)phenoxy)-N-(2-(2-hydroxyethylamino)-4,6-dimethoxypyrimidin-5-yl)thiazole-4-carboxamide;
2-(6-bromo-3,3-dimethyl-2,3-dihydro-1H-inden-5-yloxy)-N-(2-(2-(isopropylamino)ethylamino)-4,6-dimethoxypyrimidin-5-yl)thiazole-4-carboxamide;
2-(6-chloro-3,3-dimethyl-2,3-dihydro-1H-inden-5-yloxy)-N-(2-(2-(isopropylamino)ethylamino)-4,6-dimethoxypyrimidin-5-yl)thiazole-4-carboxamide;
N-(2-(2-(isopropylamino)ethylamino)-4,6-dimethoxypyrimidin-5-yl)-2-(6-methoxy-3,3-dimethyl-2,3-dihydro-1H-inden-5-yloxy)thiazole-4-carboxamide;
N-(2-(2-(isopropylamino)ethylamino)-4,6-dimethoxypyrimidin-5-yl)-2-(5-methoxy-2-methyl phenoxy)thiazole-4-carboxamide;
2-(2-bromo-5-tert-butylphenoxy)-N-(2-(2-(isopropylamino)ethylamino)-4,6-dimethoxypyrimidin-5-yl)thiazole-4-carboxamide;
2-(5-tert-butyl-2-methoxyphenoxy)-N-(2-(2-(isopropylamino)ethylamino)-4,6-dimethoxypyrimidin-5-yl)thiazole-4-carboxamide;
2-(5-bromo 2-methylphenoxy)-N-(2-(2-(isopropylamino)ethylamino)-4,6-dimethoxypyrimidin-5-yl)thiazole-4-carboxamide;
2-(4-chloro-5-isopropyl-2-methylphenoxy)-N-(2-(2-(isopropylamino)ethylamino)-4,6-dimethoxypyrimidin-5-yl)thiazole-4-carboxamide;
2-(2-tert-butylphenoxy)-N-(2-(2-(isopropylamino)ethylamino)-4,6-dimethoxypyridin-5-yl)thiazole-4-carboxamide;
N-(2-(2-(isopropylamino)ethylamino)-4,6-dimethoxypyrimidin-5-yl)-2-(2-methyl-5-propoxyphenoxy)thiazole-4-carboxamide;
2-(5-isopropoxy-2-methylphenoxy)-N-(2-(2-(isopropylamino)ethylamino)-4,6-dimethoxypyrimidin-5-yl)thiazole-4-carboxamide;
2-(5-ethoxy-2-methylphenoxy)-N-(2-(2-(isopropylamino)ethylamino)-4,6-dimethoxypyrimidin-5-yl)thiazole-4-carboxamide;
2-(5-isobutoxy-2-methylphenoxy)-N-(2-(2-(isopropylamino)ethylamino)-4,6-dimethoxypyrimidin-5-yl)thiazole-4-carboxamide;
N-(2-(2-hydroxyethylamino)-4,6-dimethoxypyrimidin-5-yl)-2-(2-methyl-5-(2-morpholinoethoxy)phenoxy)thiazole-4-carboxamide;
tert-butyl 4-(4,6-dimethoxy-5-(2-(3,3,6-trimethyl-2,3-dihydro-1H-inden-5-yloxy)thiazole-4-carboxamido)pyrimidin-2-yl)piperazine-1-carboxylate;
N-(4,6-dimethoxy-2-(piperazin-1-yl)pyrimidin-5-yl)-2-(3,3,6-trimethyl-2,3-dihydro-1H-inden-5-yloxy)thiazole-4-carboxamide;
tert-butyl 4-(4,6-dimethoxy-5-(2-(3,3,6-trimethyl-2,3-dihydro-1H-inden-5-yloxy)thiazole-4-carboxamido)pyrimidin-2-yl)-1,4-diazepane-1-carboxylate;
N-(2-(1,4-diazepan-1-yl)-4,6-dimethoxypyrimidin-5-yl)-2-(3,3,6-trimethyl-2,3-dihydro-1H-inden-5-yloxy)thiazole-4-carboxamide;
tert-butyl 2-((4,6-dimethoxy-5-(2-(3,3,6-trimethyl-2,3-dihydro-1H-inden-5-yloxy)thiazole-4-carboxamido)pyrimidin-2-ylamino)methyl)pyrrolidine-1-carboxylate;
N-(4,6-dimethoxy-2-(pyrrolidin-2-ylmethylamino)pyrimidin-5-yl)-2-(3,3,6-trimethyl-2,3-dihydro-1H-inden-5-yloxy)thiazole-4-carboxamide;
2-(5-tert-butyl-2-cyanophenoxy)-N-(2-(2-(isopropylamino)ethylamino)-4,6-dimethoxypyrimidin-5-yl)thiazole-4-carboxamide; 2-(5-(2-hydroxypropan-2-yl)-2-methylphenoxy)-N-(2-(2-(isopropylamino)ethylamino)-4,6-dimethoxypyrimidin-5-yl)thiazole-4-carboxamide;
N-(2-((1H-imidazoi-2-yl)methylamino)-4,6-dimethoxypyrimidin-5-yl)-2-(5-tert-butyl-2-methylphenoxy)thiazole-4-carboxamide;
2-(5-tert-butyl-2-chlorophenoxy)-N-(2-(2-(isopropylamino)ethylamino)-4,6-dimethoxypyrimidin-5-yl)thiazole-4-carboxamide;
5-(5-tert-butyl-2-methylphenoxy)-N-(2-(2-(isopropylamino)ethylamino)-4,6-dimethoxypyrimidin-5-yl)-4-methyl-4H-1,2,4-triazole-3-carboxamide;
5-(3-tert-butylphenoxy)-N-(2-(2-(isopropylamino)ethylamino)-4,6-dimethoxypyrimidin-5-yl)-4-methyl-4H-1,2,4-triazole-3-carboxamide;
2-(5-tert-butyl-2-methylphenoxy)-N-(4,6-dimethoxy-2-(morpholin-2-ylmethylamino)pyrimidin-5-yl)thiazole-4-carboxamide;
N-(2-(2-(N-(bis(dimethylamino)methylene)sulfamoyl)ethylamino)-4,6-dimethoxypyrimidin-5-yl)-2-(5-tert-butyl-2-methylphenoxy)thiazole-4-carboxamide;
2-(5-tert-butyl-2-methylphenoxy)-N-(2-(2-cyanoethylamino)-4,6-dimethoxypyrimidin-5-yl)thiazole-4-carboxamide;
3-(5-(2-(5-tert-butyl-2-methylphenoxy)thiazole-4-carboxamido)-4,6-dimethoxypyrimidin-2-ylamino)propanoic acid;
2-(5-tert-butyl-2-methylphenoxy)-N-(2-(2-(N-isopropylsulfamoyl)ethylamino)-4,6-dimethoxypyrimidin-5-yl)thiazole-4-carboxamide;
N-(2-(3-amino-3-oxopropylamino)-4,6-dimethoxypyrimidin-5-yl)-2-(5-tert-butyl-2-methylphenoxy)thiazole-4-carboxamide;
2-(5-tert-butyl-2-methylphenoxy)-N-(2-(2-(ethylamino)ethoxy)-4,6-dimethoxypyrimidin-5-yl)thiazole-4-carboxamide;
2-(5-tert-butyl-2-methylphenoxy)-N-(2-(2-(isopropylamino)ethoxy)-4,6-dimethoxypyrimidin-5-yl)thiazole-4-carboxamide;
2-(5-tert-butyl-2-methylphenoxy)-N-(2-(3-(ethylamino)propyl)-4,6-dimethoxypyrimidin-5-yl)thiazole-4-carboxamide;
2-(5-tert-butyl-2-methylphenoxy)-N-(2-(3-(isopropylamino)propyl)-4,6-dimethoxypyrimidin-5-yl)thiazole-4-carboxamide;
2-(5-tert-butyl-2-methylphenoxy)-N-(2-(3-(ethylamino)propylamino)-4,6-dimethoxypyrimidin-5-yl)thiazole-4-carboxamide;
2-(5-tert-butyl-2-methylphenoxy)-N-(2-(3-(isopropylamino)propylamino)-4,6-dimethoxypyrimidin-5-yl)thiazole-4-carboxamide;
2-(5-tert-butyl-2-methylphenoxy)-N-(2-(3-(ethylamino)propoxy)-4,6-dimethoxypyrimidin-5-yl)thiazole-4-carboxamide;
2 (5-tert-butyl 2-methylphenoxy)-N-(2-(3-(isopropylamino)propoxy)-4,6-dimethoxypyrimidin-5-yl)thiazole-4-carboxamide;
2-(5-tert-butyl-2-methylphenoxy)-N-(2-(4-(ethylamino)butyl)-4,6-dimethoxypyrimidin-5-yl)thiazole-4-carboxamide; and
2-(5-tert-butyl-2-methylphenoxy)-N-(2-(4-(isopropylamino)butyl)-4,6-dimethoxypyrimidin-5-yl)thiazole-4-carboxamide.

15. The compound according to any preceding claim 1, which is chiral and is in the form of a single enantiomer or diastereomer.

16. (canceled)

17. A pharmaceutical composition comprising a compound of claim 1 and a pharmaceutically acceptable diluent or carrier.

18. (canceled)

19. A method for the treatment or prevention of endometriosis, uterine myoma, an ovarian disease, a mammary cystic disease, prostatic hypertrophy, amenorrhoea, precocious puberty, premenstrual syndrome, a sex-steroid-dependent pathophysiology or benign prostatic hyperplasia, or to arrest spermatogenesis, wherein the method comprises administering, to a patient in need of such prevention or treatment, a compound of formula (I): 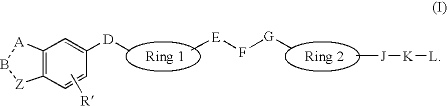
wherein
either B is absent and A and Z are the same or different and are each hydrogen, halogen, alkyl, hydroxy, alkoxy, —CN, —O(R^{c})₂OH, —N(R^{d})C(═X)R^{c}, —C(═X)N(R^{c})(R^{d}), S(O)ₘ—R^{c}, —N(R^{c})(R^{d})S(O)₂, —S(O)₂N(R^{c})(R^{d}), —N(R^{e})₂, aryl optionally substituted with R^{a} or —O-aryl optionally substituted with R^{a}; or
B is present and is —(CH₂)ₙ—, —C(R^{b})₂— or —O—, or B taken together with A or Z can be —C═C(R^{b})—, —C(R^{b})═C—, CH—CH(R^{b})— or —CH(R^{b})—CH₂—
D is —O— or —S(O)ₘ;
E is a bond or is —(CH₂)ₙ, —N(R^{d})—, —(CH₂)ₙN(R^{d})— or —N(R^{d})(CH₂)ₙ—;
F is —C(═X)—;
G is —(CH₂)ₙ—, —N(R^{d})—, —(CH₂)ₙN(R^{d})— or —N(R^{d})(CH₂)ₙ;
J is a bond or is —O—, —N(R^{c})C(═X)—, —C(═X)N(R^{c})—, —S(O)ₘ—, —N(R^{c})S(O)ₘ—, —S(O)ₘN(R^{c})—, —N(R^{e})— or —N(R^{g})(R^{h});
K is a bond or is alkylene optionally substituted with R^{b}; or K is cycloalkylene, cycloalkenylene, arylene, heterocycloalkylene, heterocycloalkylene or heteroarylene, any of which is optionally substituted with R^{a};
L is hydrogen, halogen, —N(R^{f})₂, —CN, —SO₂N(R^{a})₂, —SO₂N═C[N(R^{a})₂], —NHC(═O)NHOR^{a}, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}), —CN or a group of the formula 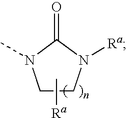
each R^{a} is the same or different and is hydrogen, halogen, alkyl, aryl, hydroxy, alkoxy, -alkoxy-(CH₂)ₙC(═O)OR^{b}, —O-aryl, —C(═X)R^{c}, —NO₂, —CN, —N(R^{c})C(═X)R^{c}, —C(═X)N(R^{c})₂, —S(O)₂N(R^{c})₂ or —N(R^{e})₂;
each R^{b} is the same or different and is hydrogen or alkyl;
each R^{c} is the same or different and is alkyl, cycloalkyl, -alkyl-aryl, -alkyl-cycloalkyl or aryl optionally substituted with R^{a};
each R^{d} is the same or different and is hydrogen or alkyl or aryl optionally with R^{a};
each R^{e} is the same or different and is hydrogen or alkyl; or R^{e} is aryl or heteroaryl, either of which is optionally substituted with R^{a};
each R^{f} is the same or different and is hydrogen or alkyl; or R^{f}—N—R^{f} taken together forms heterocycloalkyl, heterocycloalkenyl or heteroaryl, each of which is optionally substituted with R^{e};
each R^{g} is alkyl, cycloalkyl or alkyl-cycloalkyl, any of which is optionally substituted by an oxo and/or fluoro group;
each R^{h} is alkyl, cycloalkyl, or alkyl-cycloalkyl substituted with N(R^{f})₂ or R^{g} and R^{h} are taken together to form a heterocycloalkyl ring
each X is the same or different and is oxygen or sulphur;
Ring 1 is a five- or six-membered heteroaryl ring containing at least 2 heteroatoms from O, N and/or S, which is optionally substituted with one or more R^{a};
Ring 2 is arylene or heteroarylene, either of which is optionally substituted with one or more R^{a};
each m is the same or different and is 0, 1 or 2; and
each n is the same or different and is 0, 1, 2 or 3;
or a pharmaceutically acceptable salt thereof.

20. The method according to claim 19, for the treatment or prevention of endometriosis with pain, polycystic ovarian disease or secondary amenorrhoea.

21. A method for the treatment or prevention of Alzheimer's disease wherein the method comprises administering, to a patient in need of such prevention or treatment, a compound of formula (I): 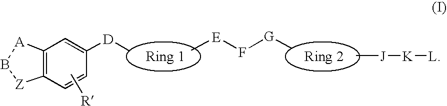
wherein
either B is absent and A and Z are the same or different and are each hydrogen, halogen, alkyl, hydroxy, alkoxy, —CN, —C(R^{c})₂OH, —N(R^{d})C(═X)R^{c}, —C(═X)N(R^{c})(R^{d})—S(O)ₘ—R^{c}, —N(R^{c})(R^{d})S(O)₂, —S(O)₂N(R^{c})(R^{d})—N(R^{e})₂, aryl optionally substituted with R^{a} or —O-aryl optionally substituted with R^{a}; or
B is present and is —(CH₂)ₙ—, —C(R^{b})₂— or —O—, or B taken together with A or Z can be —C═C(R^{b})—, —C(R^{b})═C—, —CH₂—CH(R^{b})— or —CH(R^{b})—CH₂—;
D is —O— or —S(g)ₘ
E is a bond or is —(CH₂)ₙ—, —N(R^{d})—, —(CH₂)ₙN(R^{d})— or —N(R^{d})(CH₂)ₙ;
F is —C(═X)—;
G is —(CH₂)ₙ—, —N(R^{d})—, —(CH₂)—N(R^{d})— or —N(R^{d})(CH₂)ₙ;
J is a bond or is —O—, —N(R^{c})C(═X)—, —C(═X)N(R^{c})—, —S(O)ₘ—, —N(R^{c})S(O)ₘ—S(O)ₘN(R^{c})—, —N(R^{e})— or —N(R^{g})(R^{h});
K is a bond or is alkylene optionally substituted with R^{b}; or K is cycloalkylene, cycloalkenylene, arylene, heterocycloalkylene, heterocycloalkylene or heteroarylene, any of which is optionally substituted with R^{a};
L is hydrogen, halogen, —N(R^{f})₂, —CN, —SON(R^{a})₂, —SO₂N═C[N(R^{a})₂], —NHC(═O)NHOR^{a}, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}), —CN or a group of the formula 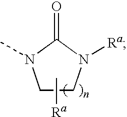
each R^{a} is the same or different and is hydrogen, halogen, alkyl, aryl, hydroxy, alkoxy, -alkoxy-(CH₂)ₙC(═O)OR^{b}, —O-aryl, —C(═X)R^{c}, —NO₂, —CN, —N(R^{c})C(═X)R^{c}, —C(═X)N(R^{c})₂, —S(O)₂N(R^{c})₂ or —N(R^{e})₂;
each R^{b} is the same or different and is hydrogen or alkyl;
each R^{c} is the same or different and is alkyl, cycloalkyl, -alkyl-aryl, -alkyl-cycloalkyl or aryl optionally substituted with R^{a};
each R^{d} is the same or different and is hydrogen or alkyl or aryl optionally with R^{a};
each R^{e} is the same or different and is hydrogen or alkyl; or R^{e} is aryl or heteroaryl, either of which is optionally substituted with R^{a};
each R^{f} is the same or different and is hydrogen or alkyl; or R^{f}—N—R^{f} taken together forms heterocycloalkyl, heterocycloalkenyl or heteroaryl, each of which is optionally substituted with R^{e};
each R^{g} is alkyl, cycloalkyl or alkyl-cycloalkyl, any of which is optionally substituted by an oxo and/or fluoro group;
each R^{h} is alkyl, cycloalkyl, or alkyl-cycloalkyl substituted with N(R^{f}) or R^{g} and R^{h} are taken together to form a heterocycloalkyl ring;
each X is the same or different and is oxygen or sulphur;
Ring 1 is a five- or six-membered heteroaryl ring containing at least 2 heteroatoms from O, N and/or S, which is optionally substituted with one or more R^{a};
Ring 2 is arylene or heteroarylene, either of which is optionally substituted with one or more R^{a};
each m is the same or different and is 0, 1 or 2; and
each n is the same or different and is 0, 1, 2 or 3;
or a pharmaceutically acceptable salt thereof.

22. A method for the treatment or prevention of HIV infection or AIDS wherein the method comprises administering, to a patient in need of such prevention or treatment, a compound of formula (I): 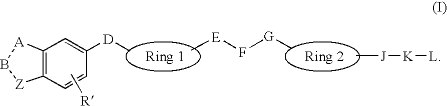
wherein
either B is absent and A and Z are the same or different and are each hydrogen, halogen, alkyl, hydroxy, alkoxy, —CN, —C(R^{c})₂OH, —N(R^{d})C(═X)R^{c}, —C(═X)N(R^{c})(R^{d}), —S(O)ₘ—R^{c}, —N(R^{c})(R^{d})₂, —S(O)₂N(R^{c})(R^{d}), —N(R^{e})₂, aryl optionally substituted with R^{a} or —O-aryl optionally substituted with R^{a}; or
B is resent and is —(CH₂)ₙ—, C(R^{b})₂ or —O— or B taken together with A or Z can be —C═C(R^{b})—, —C(R^{b})═C—, —CH₂—CH(R^{b})— or —CH(R^{b})—CH₂—;
D is —O— or —S(O)ₘ—;
E is a bond or is —(CH₂)ₙ—, —N(R^{d})—, —(CH₂)ₙN(R^{d})— or —N(R^{d})(CH₂)ₙ—;
F is —C(═X)—;
G is —(CH₂)ₙ—, —N(R^{d})—, —(OH)_N(R^{d})— or —N(R^{d})(CH₂)ₙ—;
J is a bond or is —O—, —N(R^{d})C(═X)—, —C(═X)N(R^{c})—, —S(O)ₘ—, —N(R^{c})S(O)ₘ—, —S(O)ₘN(R^{c})—, —N(R^{e})— or —N(R^{g})(R^{h});
K is a bond or is alkylene optionally substituted with R^{b}; or K is cycloalkylene, cycloalkenylene, arylene, heterocycloalkylene, heterocycloalkylene or heteroarylene, any of which is optionally substituted with R^{a};
L is hydrogen, halogen, —N(R^{f})₂, —CN, —SO₂N(R^{a})₂, —SO₂N═C[N(R^{a})₂], —NHC(═O)NHOR^{a}, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}), —CN or a group of the formula 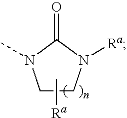
each R^{a} is the same or different and is hydrogen, halogen, alkyl, aryl, hydroxy, alkoxy, -alkoxy-(CH₂)ₙC(═O)OR^{b}, —O-aryl, —C(═X)R^{c}, —NO₂, —CN, —N(R^{c})C(═X)R^{c}, —C(═X)N(R^{c})₂, —S(O)₂N(R^{c})₂ or —N(R^{e})₂;
each R^{b} is the same or different and is hydrogen or alkyl;
each R^{c} is the same or different and is alkyl, cycloalkyl, -alkyl-aryl, -alkyl-cycloalkyl or aryl optionally substituted with R^{a};
each R^{d} is the same or different and is hydrogen or alkyl or aryl optionally with R^{a};
each R^{e} is the same or different and is hydrogen or alkyl; or R^{e} is aryl or heteroaryl, either of which is optionally substituted with R^{a};
each R^{f} is the same or different and is hydrogen or alkyl; or R^{f}—N—RF taken together forms heterocycloalkyl, heterocycloalkenyl or heteroaryl, each of which is optionally substituted with R^{e};
each R^{g} is alkyl, cycloalkyl or alkyl-cycloalkyl, any of which is optionally substituted by an oxo and/or fluoro group;
each R^{h} is alkyl, cycloalkyl, or alkyl-cycloalkyl substituted with N(R^{f})₂;
or R^{g} and R^{h} are taken together to form a heterocycloalkyl ring;
each X is the same or different and is oxygen or sulphur;
Ring 1 is a five- or six-membered heteroaryl ring containing at least 2 heteroatoms from O, N and/or S, which is optionally substituted with one or more R^{a};
Ring 2 is arylene or heteroarylene, either of which is optionally substituted with one or more R^{a};
each m is the same or different and is 0, 1 or 2; and
each n is the same or different and is 0, 1, 2 or 3;
or a pharmaceutically acceptable salt thereof.

23. A method for the treatment or prevention of a disease caused by thymic malfunction wherein the method comprises administering, to a patient in need of such prevention or treatment, a compound of formula (I): 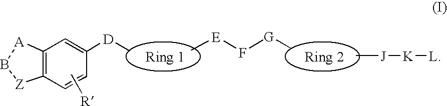
wherein
either B is absent and A and Z are the same or different and are each hydrogen, halogen, alkyl hydroxy, alkoxy, —CN, —C(R^{c})₂OH, —N(R^{d})C(═X)R^{c}, —C(═X)N(R^{c})(R^{d}), —S(O)ₘ R^{c}, —N(R^{c})(R^{d})S(O)₂, —N(R^{e})₂, aryl optionally substituted with R^{a} or —O-aryl optionally substituted with R^{a}; or
B is present and is —(CH₂)ₙ—, —C(R^{b})₂— or —O—, or B taken together with A or Z can be —C═C(R^{b})—, —C(R^{b})═C—, —CH₂CH(R^{b})— or —CH(R^{b})—CH₂
D is —O— or —S(O)ₘ—;
E is a bond or is —(CH₂)ₙ—, —N(R^{d})—, —(CH₂)ₙN(R^{d})— or —N(R^{d})(CH₂)ₙ—
F is —C(═X)—;
G is —(CH₂)ₙ—, —N(R^{d}), —(CH₂)ₙN(R^{d})— or —N(R^{d})(CH₂),
J is a bond or is —O—, —N(R^{c})C(═X)—, —C(═X)N(R^{c})—, —S(O)ₘ—, —N(R^{c})S(O)ₘ, —S(O)ₘN(R^{c})—, —N(R^{e})— or —N(R^{g})(R^{h});
K is a bond or is alkylene optionally substituted with R^{b}; or K is cycloalkylene, cycloalkenylene, arylene, heterocycloalkylene, heterocycloalkylene or heteroarylene, any of which is optionally substituted with R^{a};
L is hydrogen, halogen, —N(R^{f})_{g}, —CN, —SO₂N(R^{a})—SO₂N═C[N(R^{a})₂], —NHC(═O)NHOR^{a}, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}), —CN or a group of the formula 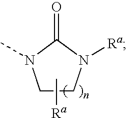
each R^{a} is the same or different and is hydrogen, halogen, alkyl, aryl, hydroxy, alkoxy, -alkoxy-(CH₂)ₙC(═O)OR^{b}, —O-aryl, —C(═X)R^{c}, —NO₂, —CN, AN(R^{c})C(═X)R^{c}, —C(═X)N(R^{c}), —S(O₂)N(R^{c})₂ or —N(R^{e})₂;
each R^{b} is the same or different and is hydrogen or alkyl;
each R^{c} is the same or different and is alkyl, cycloalkyl, -alkyl-aryl, -alkyl-cycloalkyl or aryl optionally substituted with R^{a};
each R^{d} is the same or different and is hydrogen or alkyl or aryl optionally with R^{a};
each R^{e} is the same or different and is hydrogen or alkyl; or R^{e} is aryl or heteroaryl, either of which is optionally substituted with R^{a};
each R^{f} is the same or different and is hydrogen or alkyl; or R^{f}—N—R^{f} taken together forms heterocycloalkyl, heterocycloalkenyl or heteroaryl each of which is optionally substituted with R^{e};
each R^{g} is alkyl, cycloalkyl or alkyl-cycloalkyl, any of which is optionally substituted by an oxo and/or fluoro group;
each R^{h} is alkyl, cycloalkyl, or alkyl-cycloalkyl substituted with N(R^{f})₂ or R^{g} and R^{h} are taken together to form a heterocycloalkyl ring;
each X is the same or different and is oxygen or sulphur;
Ring 1 is a five- or six-membered heteroaryl ring containing at least 2 heteroatoms from O, N and/or S, which is optionally substituted with one or more R^{a};
Ring 2 is arylene or heteroarylene, either of which is optionally substituted with one or more R^{a};
each m is the same or different and is 0, 1 or 2; and
each n is the same or different and is 0, 1, 2 or 3;
or a pharmaceutically acceptable salt thereof.

24. The method according to claim 23, for the treatment or prevention of multiple sclerosis, rheumatoid arthritis or type 1 diabetes.

25. A method for treating cancer wherein the method comprises administering, to a patient in need of such treatment a compound of formula (I): 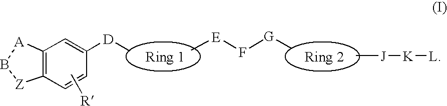
wherein
either B is absent and A and Z are the same or different and are each hydrogen, halogen, alkyl, hydroxy, alkoxy, —CN, —C(R^{c})₂OH, —N(R^{d})C(═X)R^{c}, —C(═X)N(R^{c})(R^{d}), —S(O)ₘ—R^{c}, —N(R^{c})(R^{d})S(O)₂, —S(O)₂N(R^{c})(R^{d}), —N(R^{e})₂, aryl optionally substituted with R^{a} or —O-aryl optionally substituted with R^{a}; or
B is present and is —(CH₂)ₙ—, —C(R^{b})₂— or —O—, or B taken together with A or Z can be —C═C(R^{b})—, —C(R^{b})═C—, —CH₂—CH(R^{b})— or —CH(R^{b})—CH₂—,
D is —O— or —S(O)ₘ—;
E is a bond or is —(CH₂)ₙ—, —N(R^{d})—, —(CH₂)ₙN(R^{d})— or —N(R^{d})(CH₂)ₙ—;
F is —C(═X)—;
G is —(CH₂)ₙ—, —N(R^{d})—, —(CH₂)ₙN(R^{d})— or N(R^{d})(CH₂)ₙ;
J is a bond or is —O—, —N(R^{c})C(═X)—, —C(═X)N(R^{c})—, —S(O)ₘ—, —N(R^{c})S(O)ₘ—, —S(O)ₘN(R^{c})—, —N(R^{e})— or —N(R^{g})(R^{h});
K is a bond or is alkylene optionally substituted with R^{b}; or K is cycloalkylene, cycloalkenylene, arylene, heterocycloalkylene, heterocycloalkylene or heteroarylene, any of which is optionally substituted with R^{a};
L is hydrogen, halogen, —N(R^{f})₂, —CN, —SO₂N(R^{a})₂, —SO₂N═C[N(R^{a})₂], —NHC(═O)NHOR^{a}, cycloalkyl, cycloalkenyl, aryl, heterocycloalkyl, heterocycloalkenyl or heteroaryl, any of which is optionally substituted with R^{a}, —C(═X)OR^{d}, —OH, —OR^{c}, —C(═X)N(R^{b})(R^{c}), —S(O)ₘN(R^{b})(R^{c}), —CN or a group of the formula 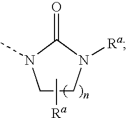
each R^{a} is the same or different and is hydrogen, halogen, alkyl, aryl, hydroxy, alkoxy, -alkoxy-(CH₂)ₙC(═O)OR^{b}, —O-aryl, —C(═X)R^{c}, —NO₂, —CN, —N(R^{c})C(═X)R^{c}, —C(═X)N(R^{c})₂, —S(O)₂N(R^{c})₂ or —N(R^{e})₂;
each R^{b} is the same or different and is hydrogen or alkyl;
each R^{c} is the same or different and is alkyl, cycloalkyl, -alkyl-aryl, -alkyl-cycloalkyl or aryl optionally substituted with R^{a};
each R^{d} is the same or different and is hydrogen or alkyl or aryl optionally with R^{a};
each R^{e} is the same or different and is hydrogen or alkyl; or R^{e} is aryl or heteroaryl, either of which is optionally substituted with R^{a};
each R^{f} is the same or different and is hydrogen or alkyl; or R^{f}—N—R^{f} taken together forms heterocycloalkyl, heterocycloalkenyl or heteroaryl, each of which is optionally substituted with R^{e};
each R^{g} is alkyl, cycloalkyl or alkyl-cycloalkyl, any of which is optionally substituted by an oxo and/or fluoro group;
each R^{h} is alkyl, cycloalkyl, or alkyl-cycloalkyl substituted with N(R^{f})₂;
or R^{g} and R^{h} are taken together to form a heterocycloalkyl ring;
each X is the same or different and is oxygen or sulphur;
Ring 1 is a five- or six-membered heteroaryl ring containing at least 2 heteroatoms from O, N and/or S, which is optionally substituted with one or more R^{a};
Ring 2 is arylene or heteroarylene, either of which is optionally substituted with one or more R^{a};
each m is the same or different and is 0, 1 or 2; and
each n is the same or different and is 0, 1, 2 or 3;
or a pharmaceutically acceptable salt thereof.
